(19) United States
(12) Patent Application Publication
(10) Pub. No.: US 2008/0045546 A1
(43) Pub. Date: Feb. 21, 2008

(54) Tetradydro-Naphthalene And Urea Derivatives

(76) Inventors: Axel Bouchon, Koln (DE); Nicole Diedrichs, Velbert (DE); Achim Hermann, Duseldorf (DE); Klemens Lustig, Wuppertal (DE); Heinrich Meier, Wuppertal (DE); Josef Pemerstorfer, Hilden (DE); Elke Reissmuller, Wuppertal (DE); Muneto Mogi, Ibaraki-ken (JP); Takeshi Yura, Nara-ken (JP); Hiroshi Fujishima, Nara-ken (JP); Masanori Seki, Pref (JP); Yuji Koriyama, Shiga-ken (JP); Kayo Yasoshima, Hyogo Pref. (JP); Keiko Misawa, Nara (JP); Masaomi Tajimi, Aichi-ken (JP); Noriyuki Yammoto, Osaka (JP); Klaus Urbahns, Lund (SE); Fumihiko Hayashi, Machida-shi (JP); Yasuhiro Tsukimi, Hyogo-ken (JP); Jang Gupta, Dusseldorf (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/575,027

(22) PCT Filed: Oct. 2, 2004
(86) PCT No.: PCT/EP04/11008

§ 371 Date: Jul. 30, 2007

(30) Foreign Application Priority Data
Oct. 15, 2003 (EP) 03023287.0
Oct. 15, 2003 (EP) 03023288.8
Nov. 8, 2003 (EP) 03025572.3
Nov. 8, 2003 (EP) 03025573.1

(51) Int. Cl. A61K 31/519; A61K 31/44; C07D 211/68; C07D 213/06

(52) U.S. Cl. 514/258.1; 514/346; 544/253; 546/292

(57) ABSTRACT

This invention relates to tetrahydro-naphthalene and urea derivatives and salts thereof which are useful as active ingredients of pharmaceutical preparations. The tetrahydro-naphthalene and urea derivatives of the present invention have vanilloid receptor (VR1) antagonistic activity, and can be used for the prophylaxis and treatment of diseases associated with VR1 activity, in particular for the treatment of urological diseases or disorders, such as detrusor overactivity (overactive bladder), urinary incontinence, neurogenic detrusor overactivity (detrusor hyperflexia), idiopathic detrusor overactivity (detrusor instability), benign prostatic hyperplasia, and lower urinary tract symptoms; chronic pain, neuropathic pain, postoperative pain, rheumatoid arthritic pain, neuralgia, neuropathies, algesia, nerve injury, ischaemia, neurodegeneration, stroke, and inflammatory disorders such as asthma and chronic obstructive pulmonary (or airways) disease (COPD).

### DETAILED DESCRIPTION OF INVENTION

1. Technical Field

The present invention relates to a tetrahydro-naphthalene or an urea derivative which is useful as an active ingredient of pharmaceutical preparations. The tetrahydro-naphthalene and urea derivatives of the present invention have vanilloid receptor (VR1) antagonistic activity, and can be used for the prophylaxis and treatment of diseases associated with VR1 activity, in particular for the treatment of urological diseases or disorders, such as detrusor overactivity (overactive bladder), urinary incontinence, neurogenic detrusor overactivity (detrusor hyperflexia), idiopathic detrusor overactivity (detrusor instability), benign prostatic hyperplasia, and lower urinary tract symptoms; chronic pain, neuropathic pain, postoperative pain, rheumatoid arthritic pain, neuralgia, neuropathies, algesia, nerve injury, ischaemia, neurodegeneration, stroke, and inflammatory disorders such as asthma and chronic obstructive pulmonary (or airways) disease (COPD).

2. Background Art

Vanilloid compounds are characterized by the presence of vanillyl group or a functionally equivalent group. Examples of several vanilloid compounds or vanilloid receptor modulators are vanillin (4-hydroxy-3-methoxy-benzaldehyde), guaiacol (2-methoxy-phenol), zingerone (4-/4-hydroxy-3-methoxyphenyl/-2-butanon), eugenol (2-methoxy4-/2-propenyl/phenol), and capsaicin (8-methy-N-vanillyl-6-noneneamide).

Among others, capsaicin, the main pungent ingredient in “hot” chili peppers, is a specific neurotoxin that desensitizes C-fiber afferent neurons. Capsaicin interacts with vanilloid receptors (VR1), which are predominantly expressed in cell bodies of dorsal root ganglia (DRG) or nerve endings of afferent sensory fibers including C-fiber nerve endings [Tominaga M, Caterina M J, Malmberg A B, Rosen T A, Gilbert H, Skinner K, Raumann B E, Basbaum A I, Julius D: The cloned capsaicin receptor integrates multiple pain-producing stimuli. Neuron. 21: 531-543, 1998]. The VR1 receptor was recently cloned [Caterina M J, Schumacher M A, Tominaga M, Rosen T A, Levine J D, Julius D: Nature 389: 816-824, (1997)] and identified as a nonselective cation channel with six transmembrane domains that is structurally related to the TRP (transient receptor potential) channel family. Binding of capsaicin to VR1 allows sodium, calcium and possibly potassium ions to flow down their concentration gradients, causing initial depolarization and release of neurotransmitters from the nerve terminals. VR1 can therefore be viewed as a molecular integrator of chemical and physical stimuli that elicit neuronal signals in pathological conditions or diseases.

There is abundant direct or indirect evidence that shows the relation between VR1 activity and diseases such as pain, ischaemia, and inflammatory disorders (e.g., WO 99/00115 and 00/50387). Further, it has been demonstrated that VR1 transduces reflex signals that are involved in the overactive bladder of patients who have damaged or abnormal spinal reflex pathways [De Groat W C: A neurologic basis for the overactive bladder. Urology 50 (6A Suppl): 36-52, 1997]. Desensitisation of the afferent nerves by depleting neurotransmitters using VR1 agonists such as capsaicin has been shown to give promising results in the treatment of bladder dysfunction associated with spinal cord injury and multiple sclerosis [(Maggi C A: Therapeutic potential of capsaicin-like molecules—Studies in animals and humans. Life Sciences 51: 1777-1781, 1992) and (DeRidder D; Chandiramani V; Dasgupta P; VanPoppel H; Baert L; Fowler C J: Intravesical capsaicin as a treatment for refractory detrusor hyperreflexia: A dual center study with long-term followup. J. Urol. 158: 2087-2092, 1997)].

It is anticipated that antagonism of the VR1 receptor would lead to the blockage of neurotransmitter release, resulting in prophylaxis and treatment of the conditions and diseases associated with VR1 activity.

It is therefore expected that antagonists of the VR1 receptor can be used for prophylaxis and treatment of the conditions and diseases including chronic pain, neuropathic pain, postoperative pain, rheumatoid arthritic pain, neuralgia, neuropathies, algesia, nerve injury, ischaemia, neurodegeneration, stroke, inflammatory disorders, urinary incontinence (UI) such as urge urinary incontinence (UUI), and/or overactive bladder.

UI is the involuntary loss of urine. UUI is one of the most common types of UI together with stress urinary incontinence (SUI) which is usually caused by a defect in the urethral closure mechanism. UUI is often associated with neurological disorders or diseases causing neuronal damages such as dementia, Parkinson's disease, multiple sclerosis, stroke and diabetes, although it also occurs in individuals with no such disorders. One of the usual causes of UUI is overactive bladder (OAB) which is a medical condition referring to the symptoms of frequency and urgency derived from abnormal contractions and instability of the detrusor muscle.

There are several medications for urinary incontinence on the market today mainly to help treating UUI. Therapy for OAB is focused on drugs that affect peripheral neural control mechanisms or those that act directly on bladder detrusor smooth muscle contraction, with a major emphasis on development of anticholinergic agents. These agents can inhibit the parasympathetic nerves which control bladder voiding or can exert a direct spasmolytic effect on the detrusor muscle of the bladder. This results in a decrease in intravesicular pressure, an increase in capacity and a reduction in the frequency of bladder contraction. Orally active anticholinergic drugs which are commonly prescribed, such as propantheline (ProBanthine), tolterodine tartrate (Detrol) and oxybutynin (Ditropan), have serious drawbacks such as unacceptable side effects such as dry mouth, abnormal visions, constipation, and central nervous system disturbances. These side effects lead to poor compliance. Dry mouth symptoms alone are responsible for a 70% non-compliance rate with oxybutynin. The inadequacies of present therapies highlight the need for novel, efficacious, safe, orally available drugs that have fewer side effects.

WO03/014064 discloses the compounds represented by the general formula:

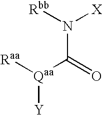

wherein
X represents C₃₋₈ cycloalkyl optionally fused by benzene, optionally substituted naphthyl, optionally substituted phenyl, optionally substituted phenyl C₁₋₆ straight alkyl, phenyl fused by cycloalykyl, etc;
Q^{aa} represents CH or N;
R^{aa} represents hydrogen or methyl;
R^{bb} represents hydrogen or methyl; and
Y represents substituted naphthyl,
   as a vanilloid receptor antagonist.

WO03/022809 discloses the compounds having vanilloid receptor antagonist activity represented by the general formula:

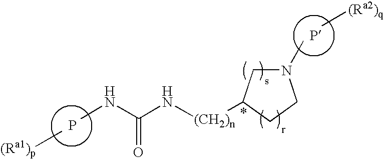

wherein
P and P′ independently represent aryl or heteroaryl;
R^{a1} and R^{a2} independently represent hydrogen, alkoxy, hydroxy, etc;
n is 0, 1, 2 or 3; p and q are independently 0, 1, 2, 3 or 4; r is 1, 2 or 3; and s is 0, 1 or 2.

WO03/053945 discloses the compounds having vanilloid receptor antagonist activity represented by the general formula:

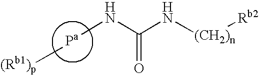

wherein
P^{a} represents phenyl, naphthyl or heterocyclyl;
n is 2, 3, 4, 5 or 6; p is independently 0, 1, 2, 3 or 4;
R^{b1} represents hydrogen, alkoxy, hydroxy, etc; and
R^{a2} represents

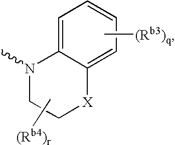

wherein X is a bond, C, O, or NR^{b8}; and r, q, R^{b3}, R^{b4} are defined in the application.

WO03/070247 discloses the compounds having vanilloid receptor antagonist activity represented by the general formula:

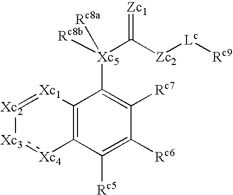

wherein
Xc₁ represents N or CR^{c1}; Xc₂ represents N or CR^{c2}; Xc₃ represents N, NR^{c3} or CR^{c3}; Xc₄ represents a bond, N or CR^{c4}; Xc₅ represents N or C; provided that at least one of Xc₁, Xc₂, Xc₃ and Xc₄ is N; Zc₁ represents O, NH or S; Zc₂ represents a bond, NH or S; L^{c} represents alkylene, cycloalkylene, etc; R^{c1}, R^{c2}, R^{c3}, R^{c4}, R^{c5}, R^{c6}, R^{c7}, R^{c8a}, R^{c8b} are defined in the application; and R^{c9} represents hydrogen, aryl, cycloalkyl, and heterocycle.

WO03/080578 discloses the compounds having vanilloid receptor antagonist activity represented by the general formula:

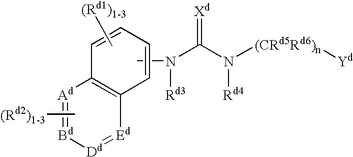

wherein
A^{d}, B^{d}, D^{d} and E^{d} are each C or N with the proviso that one or more are N; X^{d} is an O, S or ═NCN; Y^{d} is an aryl, heteroaryl, carbocyclyl or fused-carbocyclyl; n is 0, 1, 2 or 3; and R^{d1}, R^{d2}, R^{d3}, R^{d4}, R^{d5} and R^{d6} are defined in the application.

The development of a compound which has effective VR1 antagonistic activity and can be used for the prophylaxis and treatment of diseases associated with VR1 activity, in particular for the treatment of urinary incontinence, urge urinary incontinence, overactive bladder as well as pain, and/or inflammatory diseases such as asthma and COPD has been desired.

### SUMMARY OF THE INVENTION

This invention is to provide a compound of the formula (A), their tautomeric and stereoisomeric form, and salts thereof:

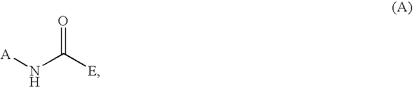

wherein
A represents the formula

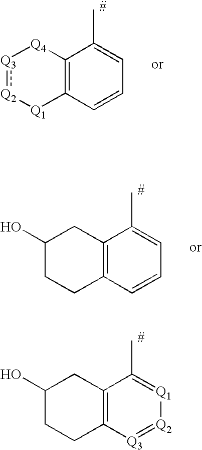

wherein
   # represents the connection position to the molecule
   and Q₁, Q₂, Q₃ and Q₄, are defined below,
      and
E represents the formula

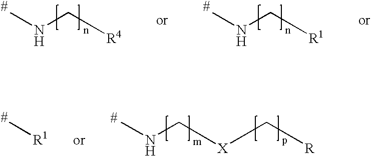

wherein
   # is represents the connection position to the molecule
   and n, m, p, X, R, R¹ and R⁴ are defined below.

### Chapter I (Summary of the Invention)

This invention is to provide an urea derivative of the formula (I), their tautomeric and stereoisomeric form, and salts thereof:

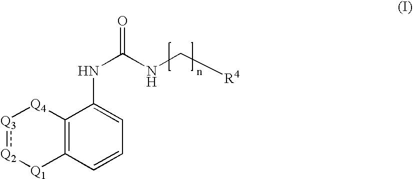

wherein
n represents an integer of 0 to 6;
Q₁ and Q₄ independently represent direct bond or methylene;
Chemical bond between [symbol]
is selected from the group consisting of a single bond and a double bond;
when [symbol]
is a single bond, Q₂ represents CHR², or CO, and Q₃ represents CHR³,
when [symbol]
is a double bond, Q₂ represents CR² and Q₃ represents CR³;
   wherein
   R² represents hydrogen, hydroxy, C₁₋₆ alkoxy or C₁₋₆ alkanoyloxy;
   R³ represents hydrogen, hydroxy, C₁₋₆ alkoxy, C₁₋₆ alkanoyloxy, or C₁₋₆ alkyl optionally substituted by hydroxy, C₁₋₆ alkoxy or C₁₋₆ alkanoyloxy,
   with the proviso that Q₁ and Q₄ can not be direct bond at the same time;
   R² and R³ can not be hydrogen at the same time;
   when Q₁ and Q₄ are both methylene and R³is hydroxy, R² is hydroxy, C₁₋₆ alkoxy or C₁₋₆ alkanoyloxy;
   when Q₁ is direct bond, R² is hydroxy, C₁₋₆ alkoxy or C₁₋₆ alkanoyloxy; and when Q₄ is direct bond, R² is hydrogen, C₁₋₆ alkoxy or C₁₋₆ alkanoyloxy;
and
   R⁴ represents aryl optionally having one or two substituents selected from the group consisting of halogen, hydroxy, C₁₋₆ alkylamino, di(C₁₋₆ alkyl)amino, C₃₋₈ cycloalkylamino, C₁₋₆ alkoxycarbonyl, phenyl, benzyl, sulfonamide, C₁₋₆ alkanoyl, C₁₋₆ alkanoylamino, carbamoyl, C₁₋₆ alkylcarbamoyl, cyano, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen, phenoxy optionally substituted by halogen or C₁₋₆ alkyl, and C₁₋₆ alkylthio optionally substituted by mono-, di-, or tri-halogen.

In another embodiment, the urea derivatives of formula (I) are those wherein;
Q₁ and Q₄ represent methylene; [symbol]
is a single bond;
Q₂ represents CHR², or CO,
   wherein
   R² represents hydroxy, C₁₋₆ alkoxy or C₁₋₆ alkanoyloxy; and
Q₃ represents CHR³,
   wherein
R³ represents hydrogen, hydroxy, C₁₋₆ alkoxy or C₁₋₆ alkanoyloxy.

In another embodiment, the urea derivatives of formula (I) are those wherein;
Q₁ represents methylene;
Q₄ represents direct bond; [symbol]
is a single bond;
Q₂ represents CHR² or CO,
   wherein
   R² represents hydrogen, C₁₋₆ alkoxy or C₁₋₆ alkanoyloxy; and
Q₃ represents CHR³,
   wherein
   R³ represents hydrogen, hydroxy, C₁₋₆ alkoxy or C₁₋₆ alkanoyloxy,
   with the proviso that R² and R³ can not be hydrogen at the same time.

In another embodiment, the urea derivatives of formula (I) are those wherein;
Q₁ represents direct bond;
Q₄ represents methylene; [symbol]
is a single bond;
Q₂ represents CHR² or CO,
   wherein
   R² represents hydroxy, C₁₋₆ alkoxy or C₁₋₆ alkanoyloxy;
Q₃ represents CHR³,
   wherein
   R³ represents hydrogen, hydroxy, C₁₋₆ alkoxy or C₁₋₆ alkanoyloxy.

In another embodiment, the urea derivatives of formula (I) are those wherein;
Q₁ and Q₄ represent methylene; [symbol]
is a double bond;
Q₂ represents CR²,
   wherein
   R² represents C₁₋₆ alkoxy or C₁₋₆ alkanoyloxy, and
Q₃ represents CR³,
   wherein
   R³ represents hydrogen, C₁₋₆ alkoxy or C₁₋₆ alkanoyloxy.

In another embodiment, the urea derivatives of formula (I) are those wherein;
Q₁ and Q₄ represent methylene; [symbol]
is a single bond or a double bond;
when [symbol]
is a single bond, Q₂ represents CH₂ and Q₃ represents CHR³,
   and when [symbol]
is a double bond, Q₂ represents CH and Q₃ represents CR³,
   wherein
   R³ represents C₁₋₆ alkyl optionally substituted by hydroxy.

Preferably, the urea derivatives of formula (I) are those wherein;
n represents an integer of 0 to 1; and
R⁴ represents phenyl optionally substituted with one or more substituents selected from the group consisting of chloro, bromo, fluoro, nitro, methoxy, trifluoromethyl and trifluoromethoxy.

More preferably, said urea derivative of the formula (I) is selected from the group consisting of:
N-[4-Chloro-3-(trifluoromethyl)phenyl]-N′-(6,7-dihydroxy-5,6,7,8-tetrahydronaphthalen-1-yl)urea;
N-[4-Chloro-3-(trifluoromethyl)phenyl]-N′-(7-hydroxy-6-methoxy-5,6,7,8-tetrahydronaphthalen-1-yl)urea
N-[4-Chloro-3-(trifluoromethyl)phenyl]-N′-(6-hydroxy-7-methoxy-5,6,7,8-tetrahydronaphthalen-1-yl)urea;
4-[({[4-Chloro-3-trifluoromethyl)phenyl]amino}carbonyl)amino]-2,3-dihydro-1H-inden-2-yl acetate;
4-[({[4-(Trifluoromethyl)benzyl]amino}carbonyl)amino]-2,3-dihydro-1H-inden-2-yl acetate;
N-[4-Chloro-3-(trifluoromethyl)phenyl]-N′-(1-hydroxy-2,3-dihydro-1H-inden-4-yl)urea;
N-[4-Chloro-3-(trifluoromethyl)phenyl]-N′-(6-hydroxy-5,6,7,8-tetrahydronaphthalen-1-yl)urea; and
N-6-Hydroxy-5,6,7,8-tetrahydronaphthalen-1-yl)-N′-[4-(trifluoromethyl)benzyl]urea.

### Chapter II (Summary of the Invention)

This invention is to provide a hydroxy-tetrahydro-naphthalene derivatives of the formula (I), their tautomeric and stereoisomeric form, and salts thereof:

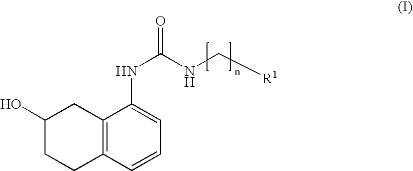

wherein
n represents an integer of 0 to 6; and
R¹ represents C₃₋₈cycloalkyl optionally fused by aryl,
   wherein
      said aryl is optionally substituted with one or more substituents selected from the group consisting of halogen, hydroxy, carboxy, nitro, cyano, amino, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen and C₁₋₆ alkylthio optionally substituted by mono-, di-, or tri-halogen;
   phenyl substituted by heteroaryl, or heteroaryloxy,
   wherein
      said heteroaryl and heteroaryloxy are optionally substituted with one or more substituents selected from the group consisting of halogen, hydroxy, carboxy, nitro, cyano, amino, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen, and C₁₋₆ alkylthio optionally substituted by mono-, di-, or tri-halogen;
   phenyl fused with heteroaryl, or heterocyclyl,
   wherein
      said heteroaryl is optionally substituted with one or more substituents selected from the group consisting of halogen, hydroxy, carboxy, nitro, cyano, amino, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆alkoxycarbonyl, or mono-, di-, or tri-halogen, C₁₋₆ alkoxy optionally substituted by mono-, di-, or tri-halogen and C₁₋₆ alkylthio optionally substituted by mono-, di-, or tri-halogen;
   or
   heteroaryl optionally substituted with one or more substituents selected from the group consisting of halogen, hydroxy, carboxy, nitro, cyano, amino, phenyl, benzyl, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen and C₁₋₆ alkylthio optionally substituted by mono-, di-, or tri-halogen.

In another embodiment, the hydroxy-tetrahydro-naphthalenylurea derivatives of formula (I) can be those wherein;
n represents an integer of 0 or 1; and
R¹ represents C₅₋₆cycloalkyl optionally fused by benzene, pyridine, or pyrimidine,
   wherein
   said benzene, pyridine, and pyrimidine are optionally substituted by halogen, nitro, or C₁₋₆ alkyl optionally substituted by mono-, di-, or tri-halogen.

In another embodiment, the hydroxy-tetrahydro-naphthalenylurea derivatives of formula (I) can be those wherein;
n represents an integer of 0 or 1; and
R¹ represents phenyl substituted by thienyl, furyl, pyrrolyl, thiazolyl, oxazolyl, isoxazolyl, imidazolyl, pyridyl, pyrimidyl, triazolthiadiazolyl, thienyloxy, furyloxy, pyrrolyl, thiazolyloxy, oxazolyloxy, isoxazolyloxy, imidazolyloxy, pyridyloxy or pyrimidyloxy,
   wherein
   said thienyl, furyl, pyrrolyl, thiazolyl, oxazolyl, isoxazolyl, imidazolyl, pyridyl, pyrimidyl, triazolthiadiazolyl, thienyloxy, furyloxy, pyrrolyl, thiazolyloxy, oxazolyloxy, isoxazolyloxy, imidazolyloxy, pyridyloxy and pyrimidyloxy are optionally substituted with one or more substituents selected from the group consisting of halogen, nitro, C₁₋₆ alkyl optionally substituted by mono-, di-, or tri-halogen, C₁₋₆ alkoxy optionally substituted by mono-, di-, or tri-halogen, and C₁₋₆ alkylthio optionally substituted by mono-, di-, or tri-halogen.

In another embodiment, the hydroxy-tetrahydro-naphthalenylurea derivatives of formula (I) can be those wherein;
n represents an integer of 0 or 1; and
R¹ represents phenyl fused with thiophene, furan, pyrrole, thiazole, oxazole, isoxazole, imidazole, pyridine, pyrimidine, 1,3-dioxalane, tetrahydrofuran, pyrrolidine, piperidine, or morpholine.
   wherein
   said thiophene, furan, pyrrole, thiazole, oxazole, isoxazole, imidazole, pyridine and pyrimidine are optionally substituted with one or more substituents selected from the group consisting of halogen, nitro, C₁₋₆ alkyl optionally substituted by mono-, di-, or tri-halogen, C₁₋₆ alkoxy optionally substituted by mono-, di-, or tri-halogen, and C₁₋₆ alkylthio optionally substituted by mono-, di-, or tri-halogen.

Preferably, the hydroxy-tetrahydro-naphthalenylurea derivatives of formula (I) can be those wherein;
n represents an integer of 0 or 1; and
R¹ represents phenyl fused with 1,3-dioxalane or tetrahydrofuran.

In another embodiment, the hydroxy-tetrahydro-naphthalenylurea derivatives of formula (I) can be those wherein;
n represents an integer of 0 or 1;
R¹ represents thienyl, furyl, pyrrolyl, thiazolyl, oxazolyl, isoxazolyl, imidazolyl, pyridyl or pyrimidyl,
   wherein
   said thienyl, furyl, pyrrolyl, thiazolyl, oxazolyl, isoxazolyl, imidazolyl, pyridyl and pyrimidyl are optionally substituted with one or more substituents selected from the group consisting of halogen, nitro, C₁₋₆ alkyl optionally substituted by mono-, di-, or tri-halogen, C₁₋₆ alkoxy optionally substituted by mono-, di-, or tri-halogen, and C₁₋₆ alkylthio optionally substituted by mono-, di-, or tri-halogen.

Preferably, the hydroxy-tetrahydro-naphthalene derivative of formula (I) are those wherein;
n represents an integer of 0 or 1; and
R¹ represents pyridyl or isoxazolyl,
   wherein
   said pyridyl and oxazolyl are optionally substituted with one or more substituents selected from the group consisting of halogen, nitro, C₁₋₆ alkyl optionally substituted by mono-, di-, or tri-halogen, C₁₋₆ alkoxy optionally substituted by mono-, di-, or tri-halogen, and C₁₋₆ alkylthio optionally substituted by mono-, di-, or tri-halogen.

More preferably, said hydroxy-tetrahydro-naphthalene derivative of the formula (I) is selected from the group consisting of:
N-(5-tert-butylisoxazol-3-yl)-N′-(7-hydroxy-5,6,7,8-tetrahydronaphthalen-1-yl)urea;
N-(2,3-dihydro-1H-inden-1-yl)-N′-(7-hydroxy-5,6,7,8-tetrahydronaphthalen-1-yl)urea;
N-(7-hydroxy-5,6,7,8-tetrahydronaphthalen-1-yl)-N-[4-(pyridin-4-yloxy)phenyl]urea;
N-(7-hydroxy-5,6,7,8-tetrahydronaphthalen-1-yl)-N′-(1,2,3,4-tetrahydronaphthalen-1-yl)urea;
N-(7-hydroxy-5,6,7,8-tetrahydronaphthalen-1-yl)-N′-[4-1,2,3-thiadiazol-4-yl)benzyl]urea;
N-(1,3-benzodioxol-5-ylmethyl)-N′-(7-hydroxy-5,6,7,8-tetrahydronaphthalen-1-yl)urea;
N-(7-hydroxy-5,6,7,8-tetrahydronaphthalen-1-yl)-N′-(3-pyridin-4-ylphenyl)urea; and
N-(7-hydroxy-5,6,7,8-tetrahydronaphthalen-1-yl)-N′-{[6-(trifluoromethyl)pyridin-3-yl]methyl}urea.

### Chapter III (Summary of the Invention)

This invention is to provide a hydroxy-tetrahydro-naphthalene derivatives of the formula (I), their tautomeric and stereoisomeric form, and salts thereof:

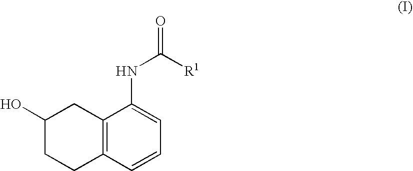

wherein
R¹ represents aryl or heteroaryl,
   wherein
   said aryl and heteroaryl are optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, trifluoromethyl, trifluoromethoxy, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle;
   C₁₋₆ alkyl optionally substituted by R¹¹, OR¹², SR¹² or N(R¹²)(R¹³),
   wherein
   R¹¹ represents aryl or heteroaryl,
      wherein
      said aryl and heteroaryl are optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle;
   R¹² represents aryl, heteroaryl, or C₁₋₆ alkyl optionally substituted by aryl or heteroaryl,
      wherein
      said aryl and heteroaryl are optionally substituted with one or more substituents selected from the group consisting of halogen, nitro, hydroxy, carboxy, amino, C₁₋₆ alkylamino, di(C₁₋₆alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
   R¹³ represents hydrogen, or C₁₋₆ alkyl;
or
C₃₋₈cycloalkyl optionally fused by aryl,
wherein
said aryl is optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)ammo, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

In another embodiment, the hydroxy-tetrahydro-naphthalenylurea derivatives of formula (I) can be those wherein;
R¹ represents phenyl, naphthyl, pyridyl, pyrimidyl, indolyl, benzofuranyl, benzothiophenyl, quinolinyl or isoquinolinyl,
   wherein
   said phenyl, naphthyl, pyridyl, pyrimidyl, indolyl, benzofuranyl, benzothiophenyl, quinolinyl and isoquinolinyl are optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

In another embodiment, the hydroxy-tetrahydro-naphthalenylurea derivatives of formula (I) can be those wherein;
R¹ represents phenyl, pyridyl, or pyrimidyl,
   wherein
   said phenyl, pyridyl, and pyrimidyl are optionally substituted by one or more of substituents selected from the group consisting of halogen, nitro, C₁₋₆ alkyl (which alkyl is optionally substituted by cyano, nitro, or mono-, di-, or tri-halogen), and C₁₋₆ alkoxy optionally substituted by mono-, di-, or tri-halogen.

In another embodiment, the hydroxy-tetrahydro-naphthalenylurea derivatives of formula (I) can be those wherein;
R¹ represents C₁₋₆ alkyl optionally substituted by R¹¹, OR¹², SR¹² or N(R¹²)(R¹³),
   wherein
   R¹¹ represents phenyl, naphthyl, pyridyl or pyrimidyl,
      wherein
      said phenyl, naphthyl, pyridyl and pyrimidyl are optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle;
   R¹² represents phenyl, naphthyl, pyridyl, pyrimidyl, or C₁₋₆ alkyl optionally substituted by phenyl, naphthyl, pyridyl or pyrimidyl,
      wherein
      said phenyl, naphthyl pyridyl and pyrimidyl are optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
   R¹³ represents hydrogen, or C₁₋₆ alkyl.

Preferably, the hydroxy-tetrahydro-naphthalenylurea derivatives of formula (I) can be those wherein;
R¹ represents C₁₋₂ alkyl optionally substituted by phenyl (which phenyl is optionally substituted with one or more substituents selected from the group consisting of halogen, nitro, C₁₋₆ alkyl optionally substituted by cyano, C₁₋₆ alkoxycarbonyl or mono-, di-, or tri-halogen, and C₁₋₆ alkoxy optionally substituted by mono-, di-, or tri-halogen), or N(R¹²)(R¹³),
   R¹² represents phenyl or C₁₋₂ alkyl optionally substituted by phenyl,
      wherein
      said phenyl is optionally substituted with one or more substituents selected from the group consisting of halogen, nitro, C₁₋₆ alkyl optionally substituted by mono-, di-, or tri-halogen, and C₁₋₆ alkoxy optionally substituted by mono-, di-, or tri-halogen; and
   R¹³ represents hydrogen, or C₁₋₆ alkyl.

In another embodiment, the hydroxy-tetrahydro-naphthalene derivative of formula (I) are those wherein;
R¹ represents C₃₋₈cycloalkyl optionally fused by phenyl,
   wherein
   said phenyl is optionally substituted with one or more substituents selected from the group consisting of halogen, nitro, C₁₋₆ alkyl optionally substituted by mono-, di-, or tri-halogen, and C₁₋₆ alkoxy optionally substituted by mono-, di-, or tri-halogen.

More preferably, said hydroxy-tetrahydro-naphthalene derivative of the formula (I) is selected from the group consisting of:
N-7-hydroxy-5,6,7,8-tetrahydronaphthalen-1-yl)-2-methoxybenzamide;
N-(7-hydroxy-5,6,7,8-tetrahydronaphthalen-1-yl)-4-(trifluoromethyl)benzamide;
5-chloro-N-(7-hydroxy-5,6,7,8-tetrahydronaphthalen-1-yl)-1H-indole-2-carboxamide;
2-(3-bromophenyl)-N-(7-hydroxy-5,6,7,8-tetrahydronaphthalen-1-yl)acetamide; and
N2-[4-chloro-3-trifluoromethyl)phenyl]-N1-(7-hydroxy-5,6,7,8-tetrahydronaphthalen-1-yl)glycinamide.

### Chapter IV (Summary of the Invention)

This invention is to provide an urea derivatives of the formula (I), their tautomeric and stereoisomeric form, and salts thereof:

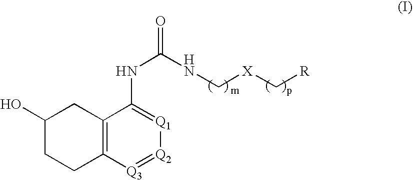

wherein
m represents 0, 1, 2, or 3;
p represents 0 or 1;
—X— represents a bond, —N— or —N(R¹)— (wherein R¹ is hydrogen or C₁₋₆ alkyl);
   with the proviso that when m is 0, —X— represents a bond.
Q₁, Q₂ and Q₃ independently represent N or CH,
   with the proviso that at least one of Q₁, Q₂ and Q₃ is N;
R represents aryl or heteroaryl,
   wherein said aryl and heteroaryl are optionally substituted with one or more substituents independently selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

In another embodiment, the urea derivatives of formula (I) can be those wherein;
m represents 0, 1, 2, or 3;
p represents 0 or 1;
—X— represents a bond, —O— or —N(R¹)— (wherein R¹ is hydrogen or C₁₋₆ alkyl);
   with the proviso that when m is 0, —X— represents a bond.
Q₁, Q₂ and Q₃ independently represent N or CH,
   with the proviso that at least one of Q₁, Q₂ and Q₃ is N;
R represents phenyl, naphthyl, pyridyl, or pyrimidyl,
   wherein
   said phenyl, naphthyl, pyridyl and pyrimidyl are optionally substituted with one or more substituents independently selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

In another embodiment, the urea derivatives of formula (I) can be those wherein;
m represents 0, 1, 2, or 3;
p represents 0 or 1;
—X— represents a bond, —O— or —N(R¹)— (wherein R¹ is hydrogen or C₁₋₆ alkyl);
   with the proviso that when m is 0, —X— represents a bond.
Q₁ represents N;
Q₂ represents CH;
Q₃ represents CH;
R represents phenyl, naphthyl, pyridyl, or pyrimidyl,
   wherein
   said phenyl, naphthyl, pyridyl and pyrimidyl are optionally substituted with one or more substituents independently selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

In another embodiment, the urea derivatives of formula (I) can be those wherein;
m represents 0, 1, 2, or 3;
p represents 0 or 1;
—X— represents a bond, —O— or —N(R¹)— (wherein R¹ is hydrogen or C₁₋₆ alkyl);
   with the proviso that when m is 0, —X— represents a bond.
Q₁ represents CH;
Q₂ represents CH;
Q₃ represents N;
R represents phenyl, naphthyl, pyridyl, or pyrimidyl,
   wherein
   said phenyl, naphthyl, pyridyl and pyrimidyl are optionally substituted with one or more substituents independently selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆-alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

In another embodiment, the urea derivatives of formula (I) can be those wherein;
m represents 0, 1, 2, or 3;
p represents 0 or 1;
—X— represents a bond, —O— or —N(R¹)— (wherein R¹ is hydrogen or C₁₋₆ alkyl); with the proviso that when m is 0, —X— represents a bond.
Q₁ represents CH;
Q₂ represents N;
Q₃ represents CH;
R represents phenyl, naphthyl, pyridyl, or pyrimidyl,
   wherein
   said phenyl, naphthyl, pyridyl and pyrimidyl are optionally substituted with one or more substituents independently selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

In another embodiment, the urea derivatives of formula (I) can be those wherein;
m represents 0, 1, 2, or 3;
p represents 0 or 1;
—X— represents a bond, —O— or —N(R¹)— (wherein R¹ is hydrogen or C₁₋₆ alkyl);
   with the proviso that when m is 0, —X— represents a bond.
Q₁ represents N;
Q₂ represents CH;
Q₃ represents N;
R represents phenyl, naphthyl, pyridyl, or pyrimidyl,
   wherein
   said phenyl, naphthyl, pyridyl and pyrimidyl are optionally substituted with one or more substituents independently selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

Preferably, the urea derivative of formula (I) are those wherein;
m represents 0;
p represents 0 or 1;
—X— represents a bond;
Q₁, Q₂ and Q₃ independently represent N or CH,
   with the proviso that at least one of Q₁, Q₂ and Q₃ is N;
R represents phenyl, naphthyl, pyridyl, or pyrimidyl,
   wherein
   said phenyl, naphthyl, pyridyl and pyrimidyl are optionally substituted with one or more substituents independently selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

Preferably, the urea derivative of formula (I) are those wherein;
m represents 0, 1, 2, or 3;
p represents 0 or 1;
—X— represents a bond, —O— or —NR¹)— (wherein R¹ is hydrogen or C₁₋₆ alkyl);
   with the proviso that when m is 0, —X— represents a bond.
Q₁, Q₂ and Q₃ independently represent N or CH,
   with the proviso that at least one of Q₁, Q₂ and Q₃ is N;
R represents phenyl, naphthyl, pyridyl, or pyrimidyl,
   wherein said phenyl, naphthyl, pyridyl, or pyrimidyl is optionally substituted by one or more of substituents selected from the group consisting of chloro, bromo, fluoro, nitro, methoxy, trifluoromethyl, trifluoromethoxy and C₁₋₆ alkanoylamino.

More preferably, said urea derivative of the formula (I) is selected from the group consisting of:
N-[4-chloro-3-trifluoromethyl)phenyl]-N′-(6-hydroxy-5,6,7,8-tetrahydroquinolin-4-yl)urea;
N-(6-hydroxy-5,6,7,8-tetrahydroquinolin-4-yl)-N′-[4-(trifluoromethyl)benzyl]urea;
N-biphenyl-3-yl-N′-(6-hydroxy-5,6,7,8-tetrahydroquinolin-4-yl)urea;
N-[4-chloro-3-(trifluoromethyl)phenyl]-N′-(7-hydroxy-5,6,7,8-tetrahydroisoquinolin-1-yl)urea;
N-(7-hydroxy-5,6,7,8-tetrahydroisoquinolin-1-yl)-N′-[4-(trifluoromethyl)benzyl]urea;
N-biphenyl-3-yl-N′-(7-hydroxy-5,6,7,8-tetrahydroisoquinolin-1-yl)urea;
N-[4-chloro-3-(trifluoromethyl)phenyl]-N′-(6-hydroxy-5,6,7,8-tetrahydroisoquinolin-4-yl)urea;
N-(6-hydroxy-5,6,7,8-tetrahydroisoquinolin-4-yl)-N′-[4-(trifluoromethyl)benzyl]urea;
N-biphenyl-3-yl-N′-(6-hydroxy-5,6,7,8-tetrahydroisoquinolin-4-yl)urea;
N-[4-chloro-3-(trifluoromethyl)phenyl]-N′-(6-hydroxy-5,6,7,8-tetrahydroquinazolin-4-yl)urea;
N-(6-hydroxy-5,6,7,8-tetrahydroquinazolin-4-yl)-N′-[4-(trifluoromethyl)benzyl]urea; and
N-biphenyl-3-yl-N′-(6-hydroxy-5,6,7,8-tetrahydroquinazolin-4-yl)urea.

### DEFINITIONS

The compounds of the present invention, their tautomeric and stereoisomeric form, and salts thereof surprisingly show excellent VR1 antagonistic activity. They are, therefore suitable especially for the prophylaxis and treatment of diseases associated with VR1 activity, in particular for the treatment of urological diseases or disorders, such as detrusor overactivity (overactive bladder), urinary incontinence, neurogenic detrusor overactivity (detrusor hyperflexia), idiopathic detrusor overactivity (detrusor instability), benign prostatic hyperplasia, and lower urinary tract symptoms.

The compounds of the present invention are also effective for treating or preventing a disease selected from the group consisting of chronic pain, neuropathic pain, postoperative pain, rheumatoid arthritic pain, neuralgia, neuropathies, algesia, nerve injury, ischaemia, neurodegeneration and/or stroke, as well as inflammatory diseases such as asthma and COPD since the diseases also relate to VR1 activity.

The compounds of the present invention are also useful for the treatment and prophylaxis of neuropathic pain, which is a form of pain often associated with herpes zoster and post-herpetic neuralgia, painful diabetic neuropathy, neuropathic low back pain, posttraumatic and postoperative neuralgia, neuralgia due to nerve compression and other neuralgias, phantom pain, complex regional pain syndromes, infectious or parainfectious neuropathies like those associated with HIV infection, pain associated with central nervous system disorders like multiple sclerosis or Parkinson disease or spinal cord injury or traumatic brain injury, and post-stroke pain.

Furthermore, the compounds of the present invention are useful for the treatment of musculo-skeletal pain, forms of pain often associated with osteoarthritis or rheumatoid arthritis or other forms of arthritis, and back pain.

In addition, the compounds of the present invention are useful for the treatment of pain associated with cancer, including visceral or neuropathic pain associated with cancer or cancer treatment.

The compounds of the present invention are furthermore useful for the treatment of visceral pain, e.g. pain associated with obstruction of hollow viscus like gallstone colik, pain associated with irritable bowel syndrome, pelvic pain, vulvodynia, orchialgia or prostatodynia, pain associated with inflammatory lesions of joints, skin, muscles or nerves, and orofascial pain and headache, e.g. migraine or tension-type headache.

Further, the present invention provides a medicament, which includes one of the compounds, described above and optionally pharmaceutically acceptable excipients.

Alkyl per se and “alk” and “alkyl” in alkenyl, alkynyl, alkoxy, alkanoyl, alkylamino, alkylaminocarbonyl, alkylaminosulfonyl, alkylsulfonylamino, alkoxycarbonyl, alkoxycarbonylamino and alkanoylamino represent a linear or branched alkyl radical having generally 1 to 6, preferably 1 to 4 and particularly preferably 1 to 3 carbon atoms representing illustratively and preferably methyl, ethyl, n-propyl, isopropyl, tert-butyl, n-pentyl and n-hexyl.

Alkoxy illustratively and preferably represents methoxy, ethoxy, n-propoxy, isopropoxy, tert-butoxy, n-pentoxy and n-hexoxy.

Alkylamino illustratively and preferably represents an alkylamino radical having one or two (independently selected) alkyl substituents, illustratively and preferably representing methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Aryl per se and in arylamino and in arylcarbonyl represents a mono- to tricyclic aromatic carbocyclic radical having generally 6 to 14 carbon atoms, illustratively and preferably representing phenyl, naphthyl and phenanthrenyl.

Cycloalkyl per se and in cycloalkylamino and in cycloalkylcarbonyl represents a cycloalkyl group having generally 3 to 8 and preferably 5 to 7 carbon atoms, illustratively and preferably representing cyclopropyl, cyclobutyl, cyclopentyl, cyclohexyl and cycloheptyl.

Heteroaryl per se and the heteroaryl portion of the heteroaralkyl, heteroaryloxy, heteroaralkyloxy, or heteroarylcarbamoyl represent an aromatic mono- or bicyclic radical having generally 5 to 10 and preferably 5 or 6 ring atoms and up to 5 and preferably up to 4 hetero atoms selected from the group consisting of S, O and N, illustratively and preferably representing thienyl, furyl, pyrrolyl, thiazolyl, oxazolyl, imidazolyl, pyridyl, pyrimidyl, pyridazinyl, indolyl, isoindolino, indazolyl, benzofuranyl, benzothiophenyl, quinolinyl, isoquinolinyl, tetrazolyl, and triazolyl.

Heterocyclyl per se and in heterocyclylcarbonyl represents a mono- or polycyclic, preferably mono- or bicyclic, nonaromatic heterocyclic radical having generally 4 to 10 and preferably 5 to 8 ring atoms and up to 3 and preferably up to 2 hetero atoms and/or hetero groups selected from the group consisting of N, O, S, SO and SO₂. The heterocyclyl radicals can be saturated or partially unsaturated. Preference is given to 5- to 8-membered monocyclic saturated heterocyclyl radicals having up to two hetero atoms selected from the group consisting of O, N and S, such as illustratively and preferably 1,3-dioxalanyl, tetrahydrofuran-2-yl, pyrrolidin-2-yl, pyrrolidin-3-yl, pyrrolinyl, piperidinyl, morpholinyl, perhydroazepinyl.

### EMBODIMENT OF THE INVENTION

### Chapter I (Embodiment of the Invention)

The compound of the formula (I) of the present invention can be, but not limited to be, prepared by combining various known methods. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3rd Edition)” by Greene and Wuts, John Wiley and Sons, New York 1999.

The compound of the formula (I) of the present invention can be, but not limited to be, prepared by the Method [A], [B], [C], [D], [E] or [F] below.

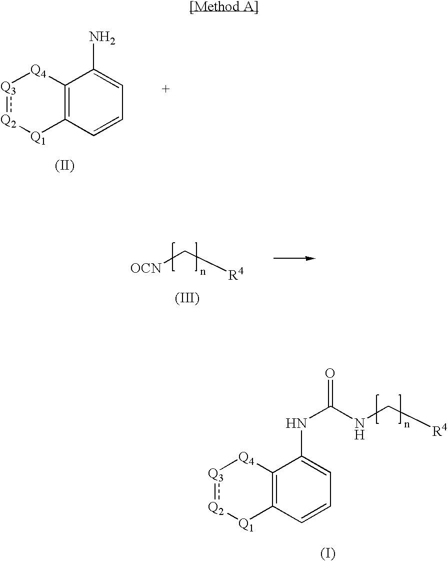

The compound of the formula (I) (wherein n, Q₁, Q₂, Q₃, Q₄ and R⁴ are the same as defined above) can be prepared by the reaction of the compound of the formula (II) (wherein Q₁, Q₂, Q₃ and Q₄ are the same as defined above) and the compound of the formula (III) (wherein n and R⁴ are the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction can be carried out in the presence of organic base such as pyridine or triethylamine.

The reaction temperature can be optionally set depending on the compounds to be reacted. The reaction temperature is usually, but not limited to, about room temperature to 100° C. The reaction may be conducted for, usually, 30 minutes to 48 hours and preferably 1 to 24 hours.

The compound (II) and (III) can be prepared by the use of known techniques or are commercially available.

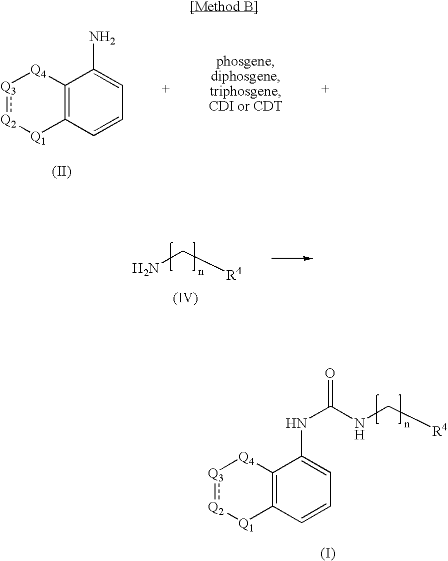

The compound of the formula (I) (wherein n, Q₁, Q₂, Q₃, Q₄ and R⁴ are the same as defined above) can be prepared by reacting the compound of the formula (II) (wherein Q₁, Q₂, Q₃ and Q₄ are the same as defined above) with phosgene, diphosgene, triphosgene, 1,1-carbonyldiimidazole (CDI), or 1,1′-carbonyldi(1,2,4-triazole)(CDT), and then adding the compound of the formula (IV) (wherein n and R⁴ are the same as defined above) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

The compound (IV) is commercially available or can be prepared by the use of known techniques and phosgene, diphosgene, triphosgene, CDI, and CDT are commercially available and

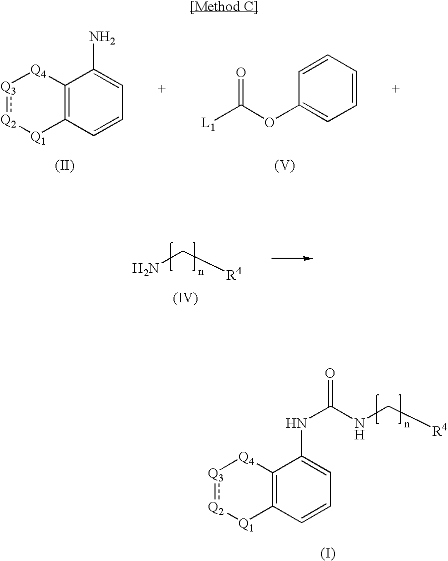

The compound of the formula (I) (wherein n, Q₁, Q₂, Q₃, Q₄ and R⁴ are the same as defined above) can be prepared by reacting the compound of the formula (II) (wherein Q₁, Q₂, Q₃ and Q₄ are the same as defined above) with the compound of the formula (V) (wherein L₁ represents halogen atom such as chlorine, bromine, or iodine atom) and then adding the compound of the formula (IV) (wherein n and R⁴ are the same as defined above) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

The reaction can be advantageously carried out in the presence of a base including, for instance, organic amines such as pyridine, triethylamine and N,N-diisopropylethylamine, dimethylaniline, diethylaniline, 4-dimethylaminopyridine, and others.

The compound (V) is commercially available or can be prepared by the use of known techniques.

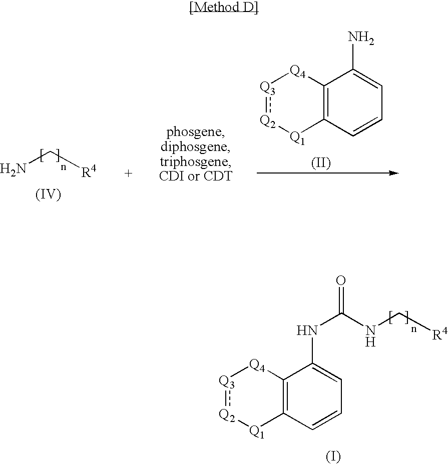

The compound of the formula (I) (wherein n, Q₁, Q₂, Q₃, Q₄ and R⁴ are the same as defined above) can be prepared by reacting the compound of the formula (IV) (wherein n and R⁴ are the same as defined above) with phosgene, diphosgene, triphosgene, 1,1-carbonyldiimidazole (CDI), or 1,1′-carbonyldi(1,2,4-triazole)(CDT) and then adding the compound of the formula (II) (wherein Q₁, Q₂, Q₃ and Q₄ are the same as defined above) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

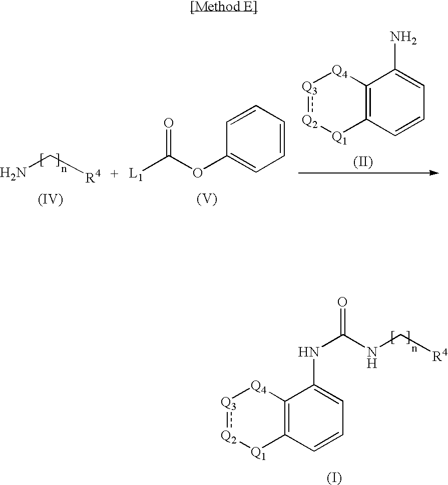

The compound of the formula (I) (wherein n, Q₁, Q₂, Q₃, Q₄ and R⁴ are the same as defined above) can be prepared by reacting the compound of the formula (IV) (wherein n and R⁴ are the same as defined above) with the compound of the formula (V) (wherein L₁ is the same as defined above) and then adding the compound of the formula (II) (wherein Q₁, Q₂, Q₃ and Q₄ are the same as defined above) to the reaction mixture. Q₁, Q₂, Q₃ and Q₄ and R⁴.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours. The reaction can be advantageously carried out in the presence of a base including, for instance, organic amines such as pyridine, triethylamine and N,N-diisopropylethylamine, dimethylaniline, diethylaniline, 4-dimethylaminopyridine, and others.

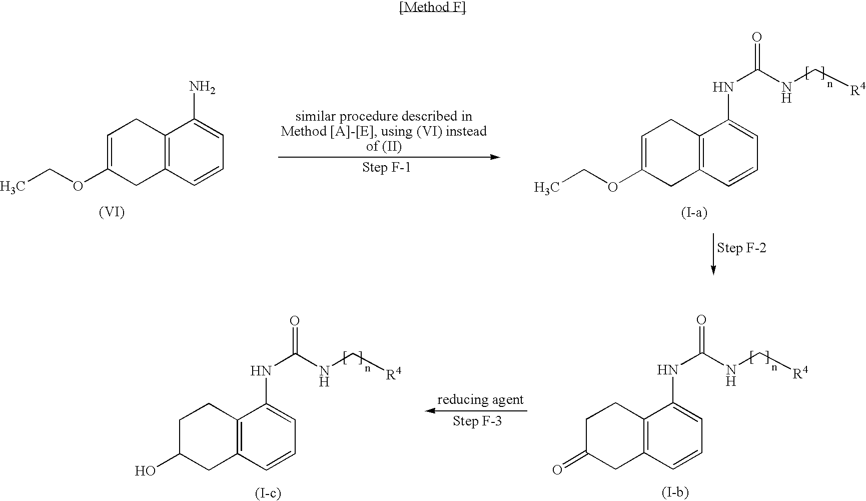

The compound of the formula (I-a), (I-b) and (I-c) (wherein n and R⁴ are the same as defined above) can be prepared by the following procedures.

In the Step F-1, the compound of the formula (I-a) (wherein n and R⁴ are the same as defined above) can be prepared in the similar manner as described in Method [A], [B], [C], [D] or [E] for the preparation of the compound of the formula (I) by using a compound of the formula (VI) instead of the compound of the formula (II).

In the Step F-2, the compound of the formula (I-b) (wherein n and R⁴ are the same as defined above) can be prepared by reacting the compound of the formula (I-a) (wherein n and R⁴ are the same as defined above) with an acid such as hydrochloric acid.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; alcohols such as methanol, ethanol; water and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 10 hours and preferably 1 to 24 hours.

In the Step F-3, the compound of the formula (I-c) (wherein n and R⁴ are the same as defined above) can be prepared by reacting the compound of the formula (I-b) (wherein n and R⁴ are the same as defined above) with reducing agent such as sodium borohydride or lithium aluminum hydride.

The reaction may be carried out in a solvent including, for instance, ethers such as diethyl ether, isopropyl ether, dioxane and tetrahydrofuran (THF) and 1,2-dimethoxyethane; alcohols such as methanol, ethanol, isopropanol, and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

The compound of the formula (VI) is commercially available or can be prepared by the use of known techniques.

### Chapter II (Embodiment of the Invention)

The compound of the formula (I) of the present invention can be, but not limited to be, prepared by combining various known methods. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3rd Edition)” by Greene and Wuts, John Wiley and Sons, New York 1999.

The compound of the formula (I) of the present invention can be, but not limited to be, prepared by the Method [A] below.

The compound of the formula (I) of the present invention can be, but not limited to be, prepared by the Method [A], [B], [C], [D], [E], [F], [G] or [H] below.

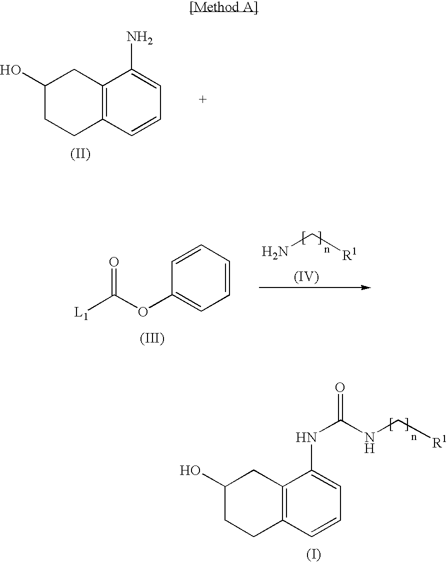

The compound of the formula (I) (wherein n and R¹ are the same as defined above) can be prepared by reacting the compound of the formula (II) and the compound of the formula (III) (wherein L₁ represents halogen atom such as chlorine, bromine, or iodine atom) and then adding the compound of the formula (V) (wherein n, R¹ are the same as defined above) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

The reaction can be advantageously carried out in the presence of a base including, for instance, organic amines such as pyridine, triethylamine and N,N-diisopropylethylamine, dimethylaniline, diethylaniline, 4-dimethylaminopyridine, and others.

The compound (III) and (IV) are commercially available or can be prepared by the use of known techniques.

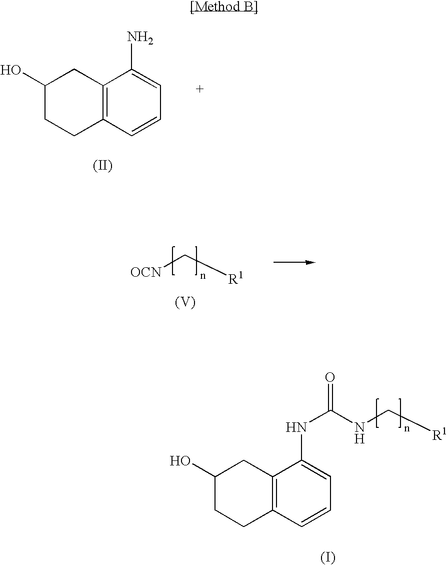

The compound of the formula (I) (wherein n and R¹ are the same as defined above) can be prepared by the reaction of the compound of the formula (II) and the compound of the formula (V) (wherein n and R¹ are the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction can be carried out in the presence of organic base such as pyridine or triethylamine.

The reaction temperature can be optionally set depending on the compounds to be reacted. The reaction temperature is usually, but not limited to, about room temperature to 100° C. The reaction may be conducted for, usually, 30 minutes to 48 hours and preferably 1 to 24 hours.

The compound (V) can be prepared by the use of known techniques or are commercially available.

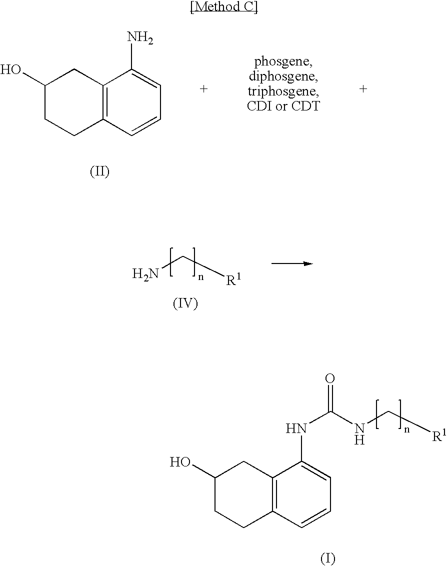

The compound of the formula (I) (wherein n and R¹ are the same as defined above) can be prepared by reacting the compound of the formula (II) with phosgene, diphosgene, triphosgene, 1,1-carbonyldiimidazole (CDI), or 1,1′-carbonyldi(1,2,4-triazole)(CDT), and then adding the compound of the formula (IV) (wherein n and R¹ are the same as defined above) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

Phosgene, diphosgene, triphosgene, CDI, and CDT are commercially available.

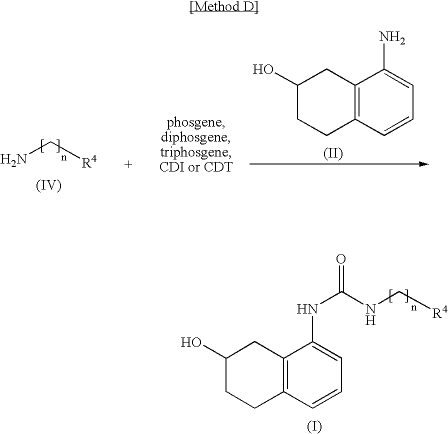

The compound of the formula (I) (wherein n and R¹ are the same as defined above) can be prepared by reacting the compound of the formula (IV) (wherein n and R¹ are the same as defined above) with phosgene, diphosgene, triphosgene, 1,1-carbonyldiimidazole (CDI), or 1,1′-carbonyldi(1,2,4-triazole)(CDT) and then adding the compound of the formula (II) (wherein R¹ is the same as defined above) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

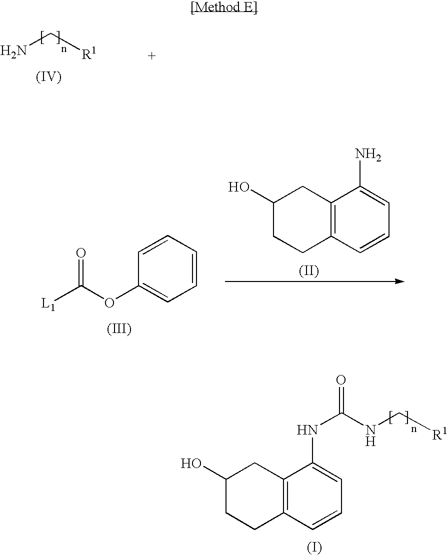

The compound of the formula (I) (wherein n and R¹ are the same as defined above) can be prepared by reacting the compound of the formula (IV) (wherein n and R¹ are the same as defined adding the compound of the formula (II) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

The reaction can be advantageously carried out in the presence of a base including, for instance, organic amines such as pyridine, triethylamine and N,N-diisopropylethylamine, dimethylaniline, diethylaniline, 4-dimethylaminopyridine, and others.

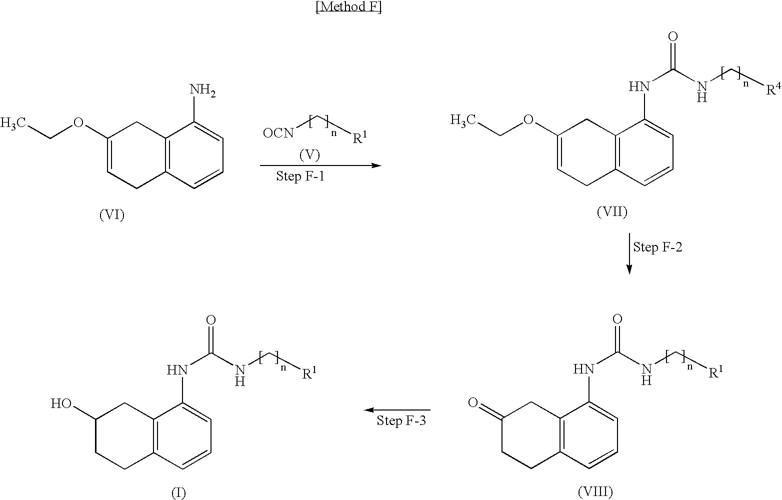

The compound of the formula (I) (wherein n and R¹ are the same as defined above) can be prepared by the following procedures in three steps;

In the Step F-1, the compound of the formula (VII) (wherein n and R¹ are the same as defined above) can be prepared by reacting the compound of the formula (VI) with the compound of the formula (V) (wherein n and R¹ are the same as defined above) in a similar manner described in Method B for the preparation of the compound of the formula (I).

In the Step F-2, the compound of the formula (VIII) (wherein n and R¹ are the same as defined above) can be prepared by reacting the compound of the formula (VII) (wherein n and R¹ are the same as defined above) with an acid such as hydrochloric acid.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; alcohols such as methanol, ethanol; water and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 10 hours and preferably 1 to 24 hours.

In the Step F-3, the compound of the formula (I) (wherein n and R¹ are the same as defined above) can be prepared by reacting the compound of the formula (VIII) (wherein n and R¹ are the same as defined above) with reducing agent such as sodium borohydride or lithium aluminum hydride.

The reaction may be carried out in a solvent including, for instance, ethers such as diethyl ether, isopropyl ether, dioxane and tetrahydrofuran (THF) and 1,2-dimethoxyethane; alcohols such as methanol, ethanol, isopropanol, and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

The compound (VI) is commercially available or can be prepared by the use of known techniques.

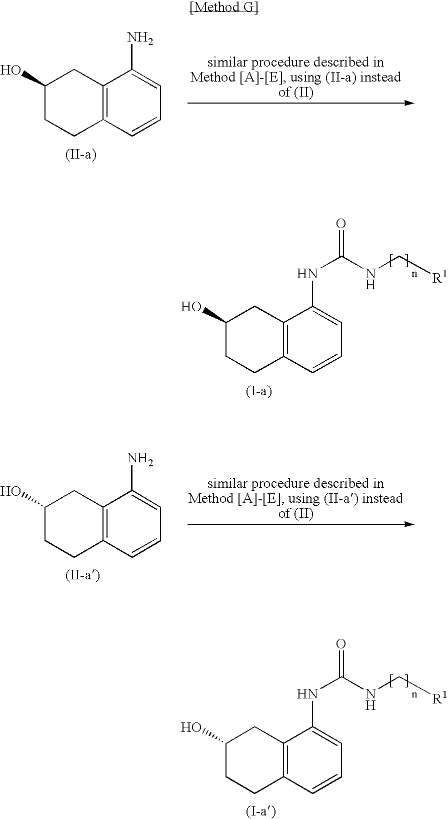

The stereoisomeric form of the compound (I), R form (I-a) (wherein n and R¹ are the same as defined above) can be prepared in the similar manner as described in Method [A], [B], [C], [D], or [E] for the preparation of the compound of the formula (I) by using a compound of the formula (II-a) instead of the compound of the formula (II).

The stereoisomeric form of the compound (I), S form (I-a′) (wherein n and R¹ are the same as defined above) can be prepared in the similar manner as described in Method [A], [B], [C], [D], or [E] for the preparation of the compound of the formula (I by using a compound of the formula (II-a′) instead of the compound of the formula (II).

The compound (II-a) or (I-a′) can be prepared by the use of known techniques.

### Chapter III (Embodiment of the Invention)

The compound of the formula (I) of the present invention can be, but not limited to be, prepared by combining various known methods. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3rd Edition)” by Greene and Wuts, John Wiley and Sons, New York 1999.

The compound of the formula (I) of the present invention can be, but not limited to be, prepared by the Method [A] below.

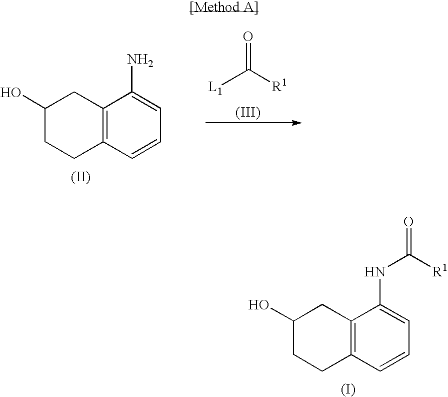

The compound of the formula (I) (wherein R¹ is the same as defined above) can be prepared by the reaction of the compound of the formula (II) with the compound of the formula (m) (wherein R¹ is the same as defined above and L₁ represents a leaving group including, for instance, hydroxy, halogen atom such as chlorine, bromine, or iodine atom, or azole such as imidazole or triazole.).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 0° C. to 50° C. The reaction may be conducted for, usually, 30 minutes to 24 hours and preferably 1 to 10 hours.

The reaction can be advantageously carried out in the presence of a base including, for instance, organic amines such as pyridine, triethylamine and N,N-diisopropylethylamine, dimethylaniline, diethylaniline, 4-dimethylaminopyridine, and others.

When L₁ is hydroxy, the reaction can be advantageously carried out using coupling agent including; for instance, hydroxybenzotriazole, carbodiimides such as N, N-dicyclohexylcarbodiimide and 1-(3-dimethyl-aminopropyl)-3-ethylcarbodiimide; carbonyldiazoles such as 1,1′-carbonyldi(1,3-imiazole)(CDI) and 1,1′-carbonyldi(1,2,4-triazole)(CDT), and the like.

The compound (II) and (III) are commercially available or can be prepared by the use of known techniques.

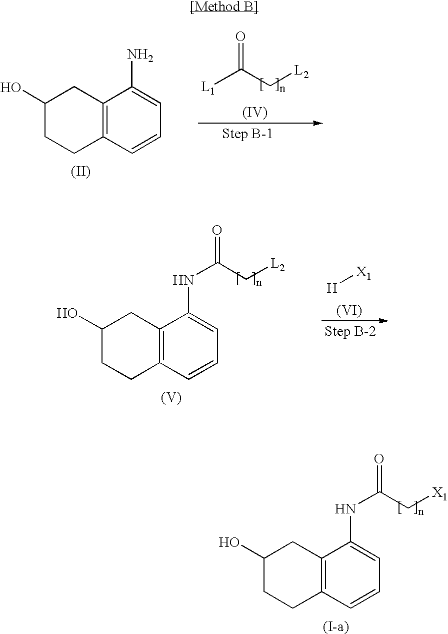

The compound of the formula (I-a) (wherein n is 1 to 6; and X₁ is OR¹², SR¹² or N(R¹²)(R¹³) (in which R¹² and R¹³ are the same as defined above)) can be, but not limited to be, prepared by the following procedures.

In Step B-1, the compound of the formula (V) (wherein n is 1 to 6; L₁ represents a leaving group including, for instance, hydroxy, halogen atom such as chlorine, bromine, or iodine atom, or azole such as imidazole or triazole; and L₂ represents a leaving group including, for instance, halogen atom such as chlorine, bromine, or iodine atom) can be prepared in a similar manner as described in Method [A] by using a compound of the formula (IV) (wherein n, L₁ and L₂ are the same as defined above) instead of the compound of the formula (III).

In Step B-2, the compound of the formula (I-a) (wherein n and X₁ are the same as defined above) can be, but not limited to be, prepared by the reaction of the compound of the formula (V) (wherein n and L₂ are the same as defined above) with the compound of the formula (VI) (wherein X₁ is the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 0° C. to 50° C. The reaction may be conducted for, usually, 30 minutes to 24 hours and preferably 1 to 10 hours.

The reaction can be advantageously carried out in the presence of a base including, for instance, organic amines such as pyridine, triethylamine and N,N-diisopropylethylamine, dimethylaniline, diethylaniline, 4-dimethylaminopyridine, and others.

The compound (IV) and (VI) are commercially available or can be prepared by the use of known techniques.

### Chapter IV (Embodiment of the Invention)

The compound of the formula (I) of the present invention can be, but not limited to be, prepared by combining various known methods. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3rd Edition)” by Greene and Wuts, John Wiley and Sons, New York 1999.

The compound of the formula (I) of the present invention can be, but not limited to be, prepared by the Method [A], [B], [C], [D], or [E] below.

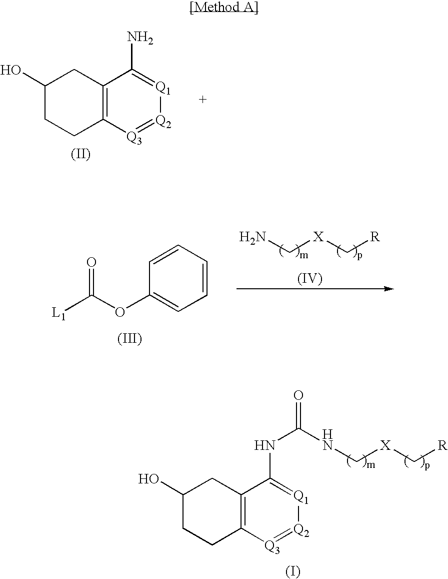

The compound of the formula (I) (wherein m, p, Q₁, Q₂, Q₃, R and X are the same as defined above) can be prepared by reacting the compound of the formula (II) (wherein Q₁, Q₂ and Q₃ are the same as defined above) and the compound of the formula (III) (wherein L₁ represents a leaving group including halogen atom such as chlorine, bromine, or iodine atom) and then adding the compound of the formula (IV) (wherein m, p, R and X are the same as defined above) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 24 hours and preferably 1 to 10 hours.

The reaction can be advantageously carried out in the presence of a base including, for instance, organic amines such as pyridine, triethylamine and N,N-diisopropylethylamine, dimethylaniline, diethylaniline, 4-dimethylaminopyridine, and others.

The compound of the formula (III) and (IV) are commercially available or can be prepared by the use of known techniques.

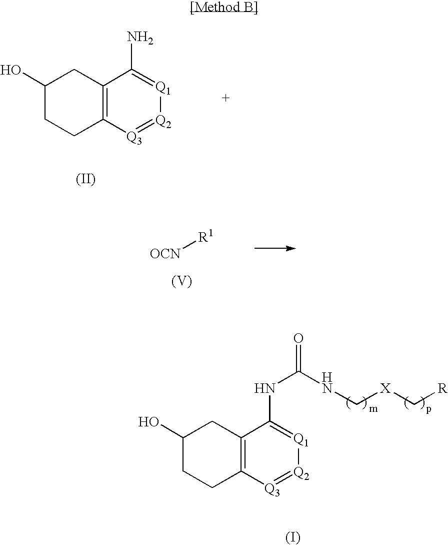

The compound of the formula (I) (wherein m, p, Q₁, Q₂, Q₃, R and X are the same as defined above) can be prepared by the reaction of the compound of the formula (II) (wherein Q₁, Q₂ and Q₃ are the same as defined above) and the compound of the formula (V) (wherein m, p, R and X are the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction can be carried out in the presence of organic base such as pyridine or triethylamine.

The reaction temperature can be optionally set depending on the compounds to be reacted. The reaction temperature is usually, but not limited to, about room temperature to 100° C. The reaction may be conducted for, usually, 30 minutes to 24 hours and preferably 1 to 10 hours.

The compound (V) can be prepared by the use of known techniques or are commercially available.

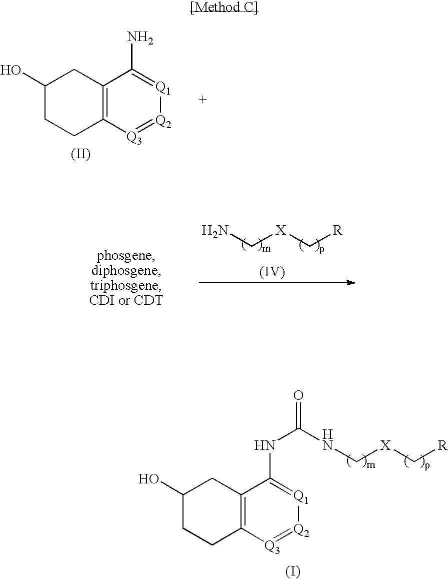

The compound of the formula (I) (wherein m, p, Q₁, Q₂, Q₃, R and X are the same as defined above) can be prepared by reacting the compound of the formula (II) (wherein Q₁, Q₂ and Q₃ are the same as defined above) with phosgene, diphosgene, triphosgene, 1,1-carbonyldiimidazole (CDI), or 1,1′-carbonyldi(1,2,4-triazole)(CDT), and then adding the compound of the formula (IV) (wherein m, p, R and X are the same as defined above) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 24 hours and preferably 1 to 10 hours.

Phosgene, diphosgene, triphosgene, CDI, and CDT are commercially available.

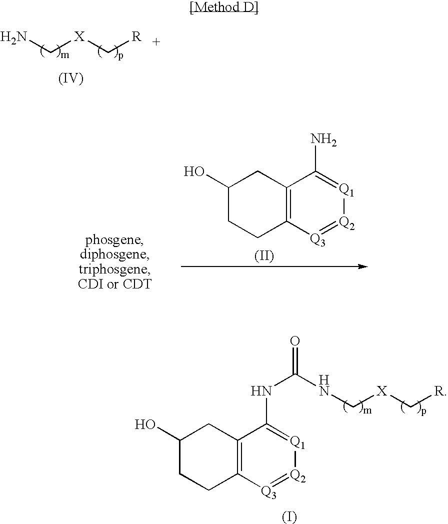

The compound of the formula (I) (wherein m, p, Q₁, Q₂, Q₃, R and X are the same as defined above) can be prepared by reacting the compound of the formula (IV) (wherein m, p, R and X are the same as defined above) with phosgene, diphosgene, triphosgene, 1,1-carbonyldiimidazole (CDI), or 1,1′-carbonyldi(1,2,4-triazole)(CDT) and then adding the compound of the formula (II) (wherein Q₁, Q₂ and Q₃ are the same as defined above) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 24 hours and preferably 1 to 10 hours.

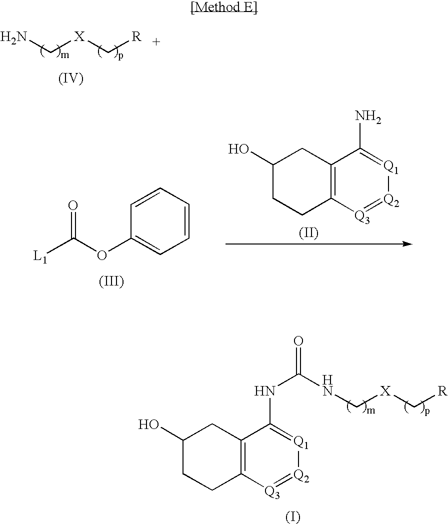

The compound of the formula (I) (wherein m, p, Q₁, Q₂, Q₃, R and X are the same as defined above) can be prepared by reacting the compound of the formula (IV) (wherein m, p, R and X are the same as defined above) and the compound of the formula (III) (wherein L₁ is the same as defined above), and then adding the compound of the formula (II) (wherein Q₁, Q₂ and Q₃ are the same as defined above) to the reaction mixture.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N, N-dimethylformamide (DMF), N, N-dimethylacetamide (DMAC) and N-methylpyrrolidone (NMP); urea such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 24 hours and preferably 1 to 10 hours.

The reaction can be advantageously carried out in the presence of a base including, for instance, organic amines such as pyridine, triethylamine and N,N-diisopropylethylamine, dimethylaniline, diethylaniline, 4-dimethylaminopyridine, and others.

### Preparation of Compound of the Formula (II)

The compound of the formula (II) (wherein Q₁, Q₂ and Q₃ are the same as defined above) can be prepared by the following procedures.

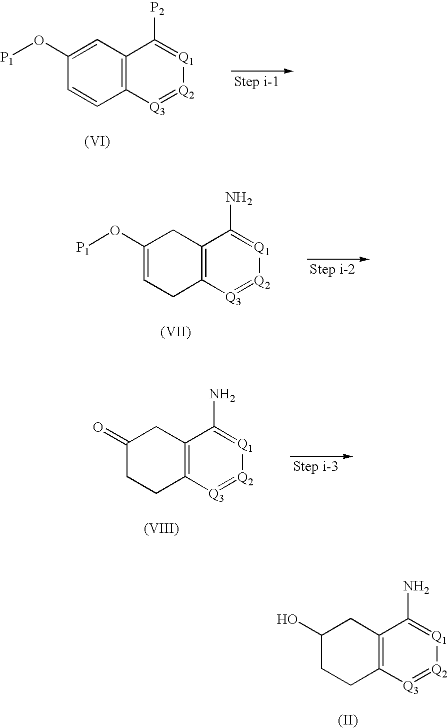

In the Step i-1, the compound of the formula (VII) (wherein Q₁, Q₂ and Q₃ are the same as defined above and P₁ represents alkyl such as methyl or ethyl) can be prepared by the reduction of the compound of the formula (VI) (wherein P₁, Q₁, Q₂ and Q₃ are the same as defined above and P₂ represents amino or nitro).

The reduction can be carried out by using the agent including, for instance, metal such as lithium, sodium, and the like.

The reaction can be carried out in a solvent including, for instance, liquid ammonia; alkylamine such as methylamine, ethylamine, and ethylenediamine (EDA); and alcohols such as methanol, ethanol, isopropanol, tert-butanol and others. Optionally, two or more of the solvents selected from the listed above can be mixed and used.

Solvent including, for instance, ethers such as diethyl ether, isopropyl ether, dioxane and tetrahydrofuran (THF) and 1,2-dimethoxyethane can be used as a co-solvent.

The reaction temperature can be optionally set depending on the compounds to be reacted. The reaction temperature is usually, but not limited to, about −78° C. to 50° C. The reaction may be conducted for, usually, 30 minutes to 24 hours and preferably 1 to 10 hours.

In the Step i-2, the compound of the formula (VIII) (wherein Q₁, Q₂ and Q₃ are the same as defined above) can be prepared by the reaction of the compound of the formula (VII) (wherein P₁, Q₁, Q₂ and Q₃ are the same as defined above are the same as defined above) with an acid such as hydrochloric acid.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; alcohols such as methanol, ethanol; water and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 24 hours and preferably 1 to 10 hours.

In the Step i-3, the compound of the formula (II) (wherein Q₁, Q₂ and Q₃ are the same as defined above) can be prepared by reacting the compound of the formula (VIII) (wherein Q₁, Q₂ and Q₃ are the same as defined above) with a reducing agent such as sodium borohydride or lithium aluminum hydride.

The reaction may be carried out in a solvent including, for instance, ethers such as diethyl ether, isopropyl ether, dioxane and tetrahydrofuran (THF) and 1,2-dimethoxyethane; alcohols such as methanol, ethanol, isopropanol, and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 24 hours and preferably 1 to 10 hours.

The compound (VI) is commercially available or can be prepared by the use of known techniques.

### Alternative Preparation Method of Compound of the Formula (VIII)

The compound of the formula (VIII) can also be prepared by the following procedures.

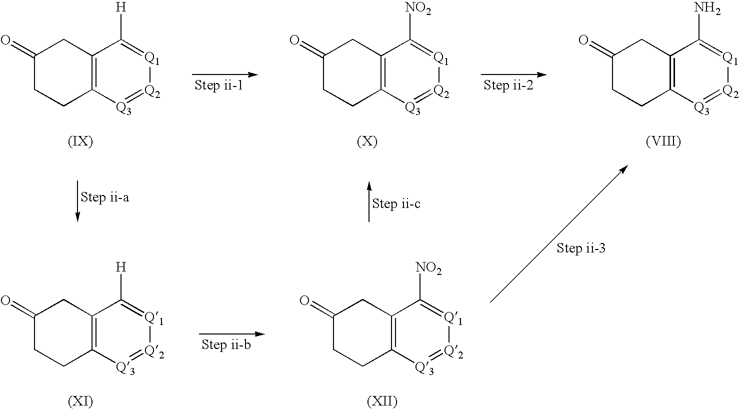

In the Step ii-1, the compound of the formula (X) (wherein Q₁, Q₂ and Q₃ are the same as defined above) can be prepared by the nitration of the compound of the formula (IX) (wherein Q₁, Q₂ and Q₃ are same as defined above.) using the agent including, for instance, nitroric acid, potassium nitrate, a combination agent of dinitrogen pentoxide and sulphur dioxide, a combination agent of dinitrogen-pentoxide, nitromethane and sodium bisulfonate, a combination agent of dimethylsulfoxide, acetic anhydride.

The reaction can be carried out without solvent or in a solvent including, for instance, acid such as acetic acid, sulfonic acid, trifluoroacetic acid. Optionally, two or more of the solvents selected from the listed above can be mixed and used.

The reaction temperature can be optionally set depending on the compounds to be reacted. The reaction temperature is usually, but not limited to, about −15° C. to 100° C. The reaction may be conducted for, usually, 30 minutes to 24 hours and preferably 1 to 10 hours.

The compound of the formula (X) (wherein Q₁, Q₂ and Q₃ are the same as defined above) can alternatively be prepared by the following procedures.

In the Step ii-a, the compound of the formula (XI) (wherein Q′₁, Q′₂ and Q′₃ independently represent N, N⁺—O⁻ or CH, with the proviso that at least one of Q₁, Q₂ and Q₃ is N⁺—O⁻) can be prepared by the oxydation of the compound of the formula (IX) (wherein Q₁, Q₂ and Q₃ are the same as defined above) using an agent including, for instance, hydrogen peroxide, m-chloroperbenzoic acid, dimethyldioxirane and the like.

The reaction can be carried out in a solvent including, for instance, halogenated hydrocarbons such as dichloromethane, chloroform and 1,2-dichloroethane; acid such as acetic acid, and water. Optionally, two or more of the solvents selected from the listed above can be mixed and used.

The reaction temperature can be optionally set depending on the compounds to be reacted. The reaction temperature is usually, but not limited to, about −15° C. to 100° C. The reaction may be conducted for, usually, 30 minutes to 24 hours and preferably 1 to 10 hours.

In the Step ii-b, the compound of the formula (XII) (wherein Q′₁, Q′₂ and Q′₃ are the same as defined above) can be prepared by the nitration of the compound of the formula (XI) (wherein Q′₁, Q′₂ and Q′₃ are the same as defined above) in a similar manner as described for the preparation of the compound of the formula (X).

In the Step ii-c, the compound of the formula (X) (wherein Q₁, Q₂ and Q₃ are the same as defined above) can be prepared by the reduction of the compound of the formula (XII) (wherein Q′₁, Q′₂ and Q′₃ are the same as defined above) using the agent including, for instance, triphenyl phosphine, triethyl phosphite, trimethyl phosphite, methanesulfonyl chloride, a combination agent of lithium chloride and sodium borohydride, and the like.

The reaction can be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene, and the like. Optionally, two or more of the solvents selected from the listed above can be mixed and used.

The reaction temperature can be optionally set depending on the compounds to be reacted. The reaction temperature is usually, but not limited to, about 0° C. to 100° C. The reaction may be conducted for, usually, 30 minutes to 24 hours and preferably 1 to 10 hours.

In the Step ii-2, the compound of the formula (VIII) (wherein Q₁, Q₂ and Q₃ are the same as defined above) can be prepared by reducing nitro group of the compound of the formula (X) (wherein Q₁, Q₂ and Q₃ are the same as defined above.) using an agent including, for instance, metals such as zinc and iron in the presence of acid including, for instance, hydrochloric acid and acetic acid and stannous chloride, or by hydrogenation using a catalyst including, for instance, palladium on carbon and platinum on carbon.

The reaction can be carried out in a solvent including, for instance, ethers such as diethyl ether, isopropyl ether, dioxane, tetrahydrofuran (THF) and 1,2-dimethoxyethane, aromatic hydrocarbons such as benzene, toluene and xylene, alcohols such as methanol, ethanol, 1-propanol, isopropanol and tert-butanol, water and others.

The reaction temperature can be optionally set depending on the compounds to be reacted. The reaction temperature is usually, but not limited to, about 20° C. to 120° C. The reaction may be conducted for, usually, 30 minutes to 24 hours and preferably 1 to 10 hours.

Alternatively, the compound of the formula (VIII) (wherein Q₁, Q₂ and Q₃ are the same as defined above) can be prepared by reduction of the compound of the formula OM (wherein Q′₁, Q′₂ and Q′₃ are the same as defined above) as shown in the Step ii-3.

The reduction can be carried out using an agent including, for instance, metals such as titanium and iron, and sodium hypophosphite together with a catalyst including, for instance, palladium on carbon and platinum on carbon.

The reaction can be carried out in a solvent including, for instance, ethers such as diethyl ether, isopropyl ether, dioxane, tetrahydrofuran (THF) and 1,2-dimethoxyethane, aromatic hydrocarbons such as benzene, toluene and xylene, alcohols such as methanol, ethanol, 1-propanol, isopropanol and tert-butanol, acid such as acetic acid, water and others.

The reaction temperature can be optionally set depending on the compounds to be reacted. The reaction temperature is usually, but not limited to, about 20° C. to 120° C. The reaction may be conducted for, usually, 30 minutes to 24 hours and preferably 1 to 10 hours.

The compound (IX) is commercially available or can be prepared by the use of known techniques.

The compound of the formula (VIII) can also be prepared by the following procedures.

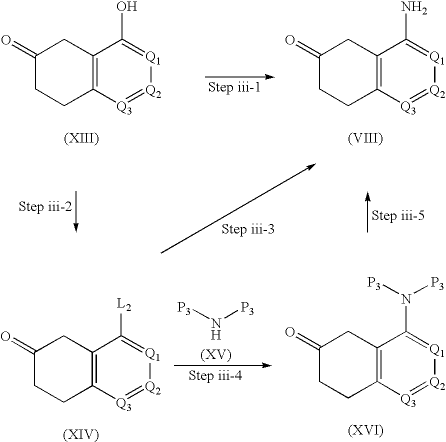

In the Step iii-1, the compound of the formula (VIII) (wherein Q₁, Q₂ and Q₃ are the same as defined above) can be prepared by the reaction of the compound of the formula (XI) (wherein Q₁, Q₂ and Q₃ are the same as defined above.) using the agent including, for instance, p-toluenesulfonyl isocyanate.

The reaction can be carried in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 24 hours and preferably 1 to 10 hours.

In the Step iii-2, the compound of the formula (XIV) (wherein Q₁, Q₂ and Q₃ are the same as defined above and L₂ represents a leaving group including halogen atom such as chlorine, bromine, or iodine atom; and alkylsulfonyloxy such as trifluoromethylsulfonyloxy) can be prepared by the reaction of the compound of the formula (XIII) (wherein Q₁, Q₂ and Q₃ are the same as defined above.) using the agent including, for instance, halogenating reagent such as POCl₃, POBr₃, PCl₅ and the like; or sulfonyl chloride such as trifluoromethylsulfonyl chloride.

The reaction may be carried out in a solvent including, for instance, halogenated hydrocarbons such as dichloromethane, chloroform and 1,2-dichloroethane such as ethers such as dioxane and tetrahydrofuran (THF) and 1,2-dimethoxyethane; aromatic hydrocarbons such as benzene, toluene, and xylene, and others. Optionally, two or more of the solvents selected from the listed above can be mixed and used.

The reaction can be advantageously conducted in the presence of a base, including, for instance, such as pyridine, triethylamine and N,N-diisopropylethylamine, dimethylaniline, diethylaniline, and others.

The reaction temperature is usually, but not limited to, about 40° C. to 200° C. and preferably about 20° C. to 180° C. The reaction may be conducted for, usually, 30 minutes to 24 hours and preferably 2 hours to 10 hours.

In the Step iii-3, the compound of the formula (XIV) (wherein L₂, Q₁, Q₂ and Q₃ are the same as defined above) can be prepared by the reaction of the compound of the formula (XIII) (wherein Q₁, Q₂ and Q₃ are the same as defined above) using the agent including, for instance, ammonia.

The reaction can be advantageously conducted in the presence of a catalyst including, for instance, copper(I) oxide, copper(II) sulfate and the like.

The reaction may be carried out in a solvent including, for instance, halogenated hydrocarbons such as dichloromethane, chloroform and 1,2-dichloroethane; such as ethers such as dioxane and tetrahydrofuran (THF) and 1,2-dimethoxyethane; aromatic hydrocarbons such as benzene, toluene, and xylene, and others. Optionally, two or more of the solvents selected from the listed above can be mixed and used.

The reaction temperature is usually, but not limited to, about 40° C. to 200° C. and preferably about 20° C. to 180° C. The reaction may be conducted for, usually, 30 minutes to 24 hours and preferably 2 hours to 12 hours.

The compound of the formula (VIII) can also be prepared by the following procedures.

In the Step iii-4, the compound of the formula (XVI) (wherein Q₁, Q₂ and Q₃ are the same as defined above; and P₃ represents aralkyl such as benzyl, 4-methoxybenzyl, 3,4-dimethoxybenzyl) can be prepared by the reaction of the compound of the formula (XIV) (wherein L₂, Q₁, Q₂ and Q₃ are the same as defined above) with the compound of the formula (XV) (wherein P₃ is the same as defined above).

The reaction can be carried out in the presence of a palladium catalyst such as tetrakis(triphenylphosphine)palladium or a combination of a phosphine ligand and a palladium catalyst such as tri-o-tolylphosphine and palladium (II) acetate.

The reaction can be advantageously carried out in the presence of a base including, for instance, cesium carbonate, sodium carbonate, potassium carbonate, barium hydroxide sodium methoxide, sodium ethoxide, potassium tert-butoxide and the like.

This reaction can be carried out in a solvent including, for instance, alcohol such as methanol, ethanol, 1-propanol, isopropanol and tert-butanol; ethers, such as dioxane, isopropyl ether, diethyl ether, 1,2-dimethoxyethane and tetrahydrofuran (THF); aromatic hydrocarbons such as benzene, toluene and xylene; nitriles such as acetonitrile; amides such as dimethylformamide (DMF) N, N-dimethylacetamide and N-methylpyrrolidone; sulfoxides such as dimethylsulfoxide (DMSO); water and others. Optionally, two or more of the solvents selected from the listed above can be mixed and used.

The reaction temperature can be optionally set depending on the compounds to be reacted. The reaction temperature is usually, but not limited to, about 10° C. to 200° C. and preferably about 50° C. to 150° C. The reaction may be conducted for, usually, 30 minutes to 24 hours and preferably 1 to 12 hours.

In the Step iii-5, the compound of the formula (VIII) (wherein Q₁, Q₂ and Q₃ are the same as defined above) can be prepared by the removal of P₃ of the compound of the formula (XVI) (wherein P₃, Q₁, Q₂ and Q₃ are the same as defined above).

The removal of P₃ can be done by hydrogenation using a catalyst including, for instance, palladium on carbon and palladium hydroxide. Also, the removal can be done by using a reagent including, for instance, trifluoroacetic acid, ceric ammonium nitrate (CAN) or 2,3-dichloro-5,6-dicyano-1,4-benzoquinone (DDQ), when P₃ is 4-methoxybenzyl or 3,4-dimethoxybenzyl.

This reaction can be carried out in a solvent including, for instance, alcohol such as methanol, ethanol, 1-propanol, isopropanol and tert-butanol; ethers, such as dioxane, isopropyl ether, diethyl ether, 1,2-dimethoxyethane and tetrahydrofuran (THF); aromatic hydrocarbons such as benzene, toluene and xylene; ester such as ethyl acetate; water and others. Optionally, two or more of the solvents selected from the listed above can be mixed and used.

The compound (XIII) and (XV) are commercially available or can be prepared by the use of known techniques.

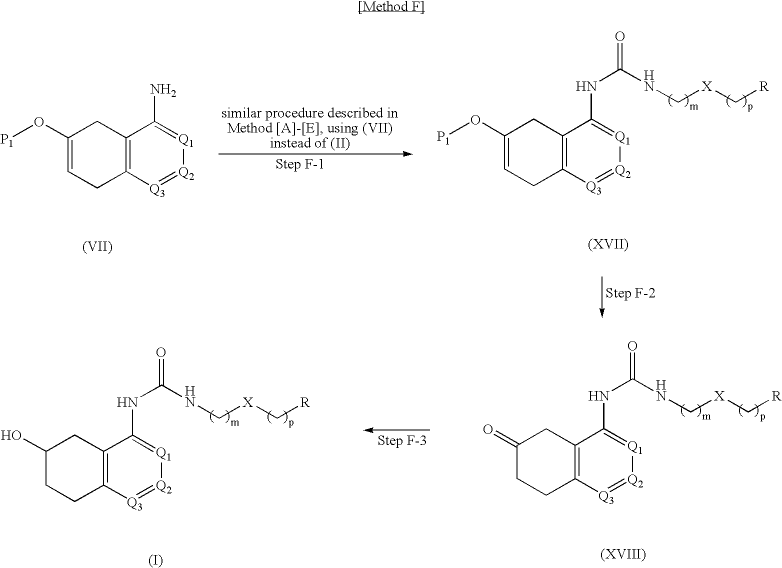

The compound of the formula (I) (wherein m, p, Q₁, Q₂, Q₃, R and X are the same as defined above) can alternatively be prepared by the following procedures in three steps;

In the Step F-1, the compound of the formula (XVII) (wherein m, p, P₁, Q₁, Q₂, Q₃, R and X are the same as defined above) can be prepared in a similar manner as described in Method [A], [B], [C], [D] or [E] for the preparation of the compound of the formula (I) by using a compound of the formula (VII) (wherein P₁, Q₁, Q₂ and Q₃ are the same as defined above) instead of the compound of the formula (II).

In the Step F-2, the compound of the formula (XVIII) (m, p, Q₁, Q₂, Q₃, R and X are the same as defined above) can be prepared by reacting the compound of the formula (XVII) (m, p, P₁, Q₁, Q₂, Q₃, R and X are the same as defined above) with an acid such as hydrochloric acid.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; alcohols such as methanol, ethanol; water and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 24 hours and preferably 1 to 10 hours.

In the Step F-3, the compound of the formula (I) (wherein m, p, Q₁, Q₂, Q₃, R and X are the same as defined above) can be prepared by reacting the compound of the formula (XVIII) (wherein m, p, Q₁, Q₂, Q₃, R and X are the same as defined above) with reducing agent such as sodium borohydride or lithium aluminum hydride.

The reaction may be carried out in a solvent including, for instance, ethers such as diethyl ether, isopropyl ether, dioxane and tetrahydrofuran (THF) and 1,2-dimethoxyethane; alcohols such as methanol, ethanol, isopropanol, and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 24 hours and preferably 1 to 10 hours.

Alternative Preparation Method of Compound of the Formula (XVIII)

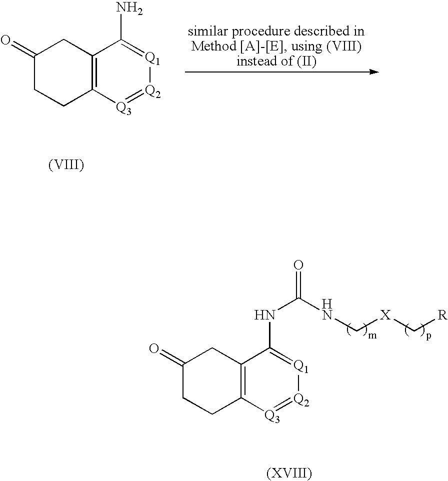

The compound of the formula (XVIII) (m, p, Q₁, Q₂, Q₃, R and X are the same as defined above) can alternatively be prepared in a similar manner as described in Method [A], [B], [C], [D] or [E] for the preparation of the compound of the formula (I) by using a compound of the formula (VIII) (wherein Q₁, Q₂ and Q₃ are the same as defined above) instead of the compound of the formula (II).

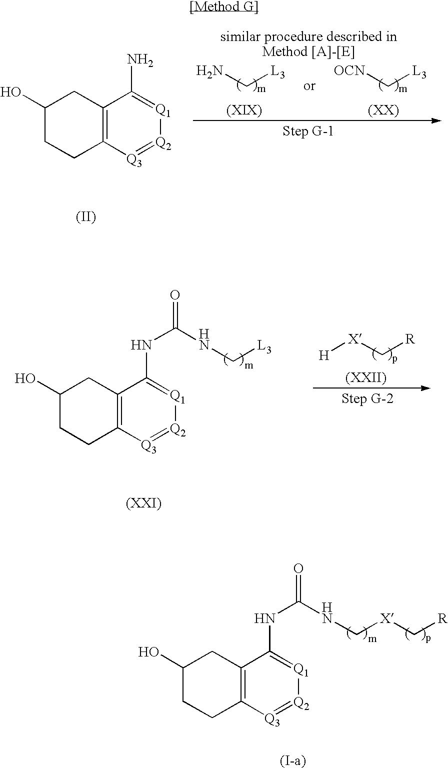

The compound of the formula (I-a) (wherein m, p, Q1, Q₂, Q₃ and R are the same as defined above and X′ is —O—, or N(R¹)—) can be prepared by the following procedures.

In the Step G-1, the compound of the formula (XXI) (wherein m, Q₁, Q₂ and Q₃ are the same as defined above and L₃ represents leaving group including, for instance, halogen atom such as chlorine, bromine, or iodine atom) can be prepared in a similar manner as described in Method [A], [B], [C], [D] or [E] for the preparation of the compound of the formula (I) by using a compound of the formula (XIX) (wherein m and L₃ are the same as defined above) instead of the compound of the formula (IV), or using a compound of the formula (XX) (wherein m and L₃ are the same as defined above) instead of the compound of the formula (V).

In the Step G-2, the compound of the formula (I-a) (wherein m, p, Q₁, Q₂, Q₃, R and X′ are the same as defined above) can be prepared by reacting the compound of the formula (XXI) (wherein m, L₃, Q, Q₂ and Q₃ are the same as defined above) and the compound of the formula (XXI) (wherein p, R and X′ are the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone (NMP); ureas such as 1,3-dimethyl-2-imidazolidinone (DMI); sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 0° C. to 50° C. The reaction may be conducted for, usually, 30 minutes to 24 hours and preferably 1 to 10 hours.

The reaction can be advantageously carried out in the presence of a base including, for instance, organic amines such as pyridine, triethylamine and N,N-diisopropylethylamine, dimethylaniline, diethylaniline, 4-dimethylaminopyridine, and others.

The compound (XIX), (XX) and (XXII) are commercially available or can be prepared by the use of known techniques.

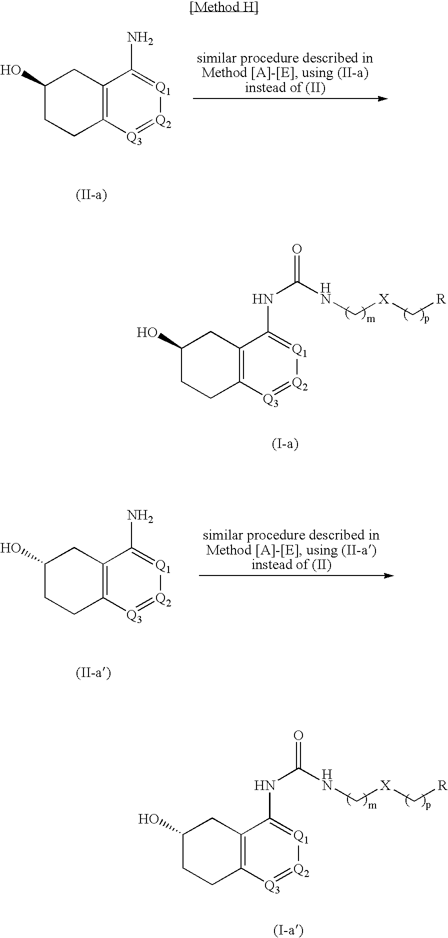

The stereoisomeric form of the compound (I), R form (I-a) (wherein m, p, Q₁, Q₂, Q₃, R and X are the same as defined above) can be prepared in a similar manner as described in Method [A], [B], [C], [D], or [E] for the preparation of the compound of the formula (I) by using a compound of the formula (II-a) (wherein Q₁, Q₂ and Q₃ are the same as defined above) instead of the compound of the formula (II).

The stereoisomeric form of the compound (I), S form (I-a′) (wherein m, p, Q₁, Q₂, Q₃, R and X are the same a defined above) can be prepared in the similar manner as described in Method [A], [B], [C], [D], or [E] for the preparation of the compound of the formula (I) by using a compound of the formula (II-a′) (wherein Q₁, Q₂ and Q₃ are the same as defined above) instead of the compound of the formula (II).

The compound (II-a) or (II-a′) can be prepared by the use of known techniques.

### SALTS AND FORMULATIONS

When the compound shown by the formula (A) or a salt thereof has an asymmetric carbon in the structure, their optically active compounds and racemic mixtures are also included in the scope of the present invention.

Typical salts of the compound shown by the formula (A) include salts prepared by reaction of the compounds of the present invention with a mineral or organic acid, or an organic or inorganic base. Such salts are known as acid addition and base addition salts, respectively.

Acids to form acid addition salts include inorganic acids such as, without limitation, sulfuric acid, phosphoric acid, hydrochloric acid, hydrobromic acid, hydriodic acid and the like, and organic acids, such as, without limitation, p-toluenesulfonic acid, methanesulfonic acid, oxalic acid, p-bromophenylsulfonic acid, carbonic acid, succinic acid, citric acid, benzoic acid, acetic acid, and the like.

Base addition salts include those derived from inorganic bases, such as, without limitation, ammonium hydroxide, alkaline metal hydroxide, alkaline earth metal hydroxides, carbonates, bicarbonates, and the like, and organic bases, such as, without limitation, ethanolamine, triethylamine, tris(hydroxymethyl)aminomethane, and the like. Examples of inorganic bases include sodium hydroxide, potassium hydroxide, potassium carbonate, sodium carbonate, sodium bicarbonate, potassium bicarbonate, calcium hydroxide, calcium carbonate, and the like.

The compound of the present invention or a salt thereof, depending on its substituents, may be modified to form lower alkylesters or known other esters; and/or hydrates or other solvates. Those esters, hydrates, and solvates are included in the scope of the present invention.

The compound of the present invention may be administered in oral forms, such as, without limitation normal and enteric coated tablets, capsules, pills, powders, granules, elixirs, tinctures, solution, suspensions, syrups, solid and liquid aerosols and emulsions. They may also be administered in parenteral forms, such as, without limitation, intravenous, intraperitoneal, subcutaneous, intramuscular, and the like forms, well-known to those of ordinary skill in the pharmaceutical arts. The compounds of the present invention can be administered in intranasal form via topical use of suitable intranasal vehicles, or via transdermal routes, using transdermal delivery systems well-known to those of ordinary skilled in the art.

The dosage regimen with the use of the compounds of the present invention is selected by one of ordinary skill in the arts, in view of a variety of factors, including, without limitation, age, weight, sex, and medical condition of the recipient, the severity of the condition to be treated, the route of administration, the level of metabolic and excretory function of the recipient, the dosage form employed, the particular compound and salt thereof employed.

The compounds of the present invention are preferably formulated prior to administration together with one or more pharmaceutically-acceptable excipients. Excipients are inert substances such as, without limitation carriers, diluents, flavoring agents, sweeteners, lubricants, solubilizers, suspending agents, binders, tablet disintegrating agents and encapsulating material.

Yet another embodiment of the present invention is pharmaceutical formulation comprising a compound of the invention and one or more pharmaceutically-acceptable excipients that are compatible with the other ingredients of the formulation and not deleterious to the recipient thereof. Pharmaceutical formulations of the invention are prepared by combining a therapeutically effective amount of the compounds of the invention together with one or more pharmaceutically-acceptable excipients therefore. In making the compositions of the present invention, the active ingredient may be mixed with a diluent, or enclosed within a carrier, which may be in the form of a capsule, sachet, paper, or other container. The carrier may serve as a diluent, which may be solid, semi-solid, or liquid material which acts as a vehicle, or can be in the form of tablets, pills powders, lozenges, elixirs, suspensions, emulsions, solutions, syrups, aerosols, ointments, containing, for example, up to 10% by weight of the active compound, soft and hard gelatin capsules, suppositories, sterile injectable solutions and sterile packaged powders.

For oral administration, the active ingredient may be combined with an oral, and non-toxic, pharmaceutically-acceptable carrier, such as, without limitation, lactose, starch, sucrose, glucose, sodium carbonate, mannitol, sorbitol, calcium carbonate, calcium phosphate, calcium sulfate, methyl cellulose, and the like; together with, optionally, disintegrating agents, such as, without limitation, maize, starch, methyl cellulose, agar bentonite, xanthan gum, alginic acid, and the like; and optionally, binding agents, for example, without limitation, gelatin, natural sugars, beta-lactose corn sweeteners, natural and synthetic gums, acacia, tragacanth, sodium alginate, carboxymethylcellulose, polyethylene glycol, waxes, and the like; and, optionally, lubricating agents, for example, without limitation, magnesium stearate, sodium stearate, stearic acid, sodium oleate, sodium benzoate, sodium acetate, sodium chloride, talc, and the like.

In powder forms, the carrier may be a finely divided solid which is in admixture with the finely divided active ingredient. The active ingredient may be mixed with a carrier having binding properties in suitable proportions and compacted in the shape and size desired to produce tablets.

The powders and tablets preferably contain from about 1 to about 99 weight percent of the active ingredient which is the novel composition of the present invention. Suitable solid carriers are magnesium carboxymethyl cellulose, low melting waxes, and cocoa butter.

Sterile liquid formulations include suspensions, emulsions, syrups and elixirs. The active ingredient can be dissolved or suspended in a pharmaceutically acceptable carriers, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in a suitable oil.

The formulation may be in unit dosage form, which is a physically discrete unit containing a unit dose, suitable for administration in human or other mammals. A unit dosage form can be a capsule or tablets, or a number of capsules or tablets. A “unit dose” is a predetermined quantity of the active compound of the present invention, calculated to produce the desired therapeutic effect, in association with one or more excipients. The quantity of active ingredient in a unit dose may be varied or adjusted from about 0.1 to about 1000 milligrams or more according to the particular treatment involved.

Typical oral dosages of the present invention, when used for the indicated effects, will range from about 0.01 mg/kg/day to about 100 mg/kg/day, preferably from 0.1 mg/kg/day to 30 mg/kg/day, and most preferably from about 0.5 mg/kg/day to about 10 mg/kg/day. In the case of parenteral administration, it has generally proven advantageous to administer quantities of about 0.001 to 100 mg /kg/day, preferably from 0.01 mg/kg/day to 1 mg/kg/day. The compounds of the present invention may be administered in a single daily dose, or the total daily dose may be administered in divided doses, two, three, or more times per day. Where delivery is via transdermal forms, of course, administration is continuous.

### EXAMPLES

The present invention will be described as a form of examples, but they should by no means be construed as defining the metes and bounds of the present invention.

In the examples below, all quantitative data, if not stated otherwise, relate to percentages by weight.

### Liquid Chromatography-Mass Spectroscopy (LC-MS)

Micromass Platform LC with Shimadzu Phenomenex ODS column (4.6 mm×30 mm) flushing a mixture of acetonitrile-water (9:1 to 1:9) at 1 ml/min of the flow rate. Mass spectra were obtained using electrospray (ES) ionization techniques.

### High Pressure Liquid Chromatography (HPLC): Method A

Instrument: Hewlett Packard series; Column Temperature: 40° C.; Mobile Phase: Water and Acetonitrile (each of them contains 10 mM ammonium acetate); Column: Phenomenex Luna 3u C18(2) (4.6 mm×30 mm); Flow Rate: 1.0 mL/min; Gradient:Time (minutes):(Water/Acetonitrile) 0 min:9/1, 0.1 min:9/1, 1.5 min: 1/9, 3.5 min:1/9, 4.5 min:9/1.

### High Pressure Liquid Chromatography (HPLC): Method B

Instrument: HP 1100 with DAD-detection; column: Kromasil RP-18, 60 mm×2 mm, 3.5 μm; eluent A: 5 ml HClO₄/l water, eluent B: acetonitrile; gradient: 0 min 2% B, 0.5 min 2% B, 4.5 min 90% B, 6.5 min 90% B; flow rate: 0.75 ml/min; oven temp.: 30° C.; UV-detection: 210 nm.

### Liquid Chromatography-Mass Spectroscopy (LC-MS): Method C

Instrument: Micromass Platform ZQ with HPLC Waters Alliance 2795; Column: Phenomenex Synergi 2μ Hydro-RP Mercury 20 mm×4 mm; eluent A: 1 l water+0.5 ml 50% aqueous formic acid, eluent B: 1 l acetonitrile+0.5 ml aqueous formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven temp.: 50° C.; UV-detection: 210 nm.

### High Pressure Liquid Chromatography (HPLC): Method D

Instrument: BP 1100 with DAD-Detection; column: Kromasil 100-RP-18, 60 mm×2.1 mm, 3.5 μm; eluent A: 5 ml HClO₄/l water, eluent B: acetonitrile; Gradient: 0 min 2% B; 0.5 min 2% B; 4.5 min 90% B; 9 min 90% B; 9.2 min 2% B; 10 min 2% B; flow rate: 0.75 ml/min; oven temp.: 30° C.; UV-detection: 210 nm.

### Liquid Chromatography-Mass Spectroscopy (LC-MS): Method E

Instrument: Micromass Platform LCZ with HPLC Agilent Series 1100; Column: Phenomenex Synergi 2μ Hydro-RP Mercury 20 mm×4 mm; eluent A: 1 l water+0.5 ml 50% aqueous formic acid, eluent B: 1 l acetonitrile+0.5 ml aqueous formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven temp.: 50° C.; UV-detection: 210 nm.

### Liquid Chromatography-Mass Spectroscopy (LC-MS): Method F

Instrument MS: Micromass ZQ; instrument HPLC: Waters Alliance 2795; Column: Merck Chromolith SpeedROD RP-18e 50 mm×4.6 mm; eluent A: water+500 μl 50% aqueous formic acid/l; eluent B: acetonitrile+500 μl 50% aqueous formic acid/l; gradient: 0.0 min 10% B→3.0 min 95% B→4.0 min 95% B; oven temp.: 35° C.; flow rate: 0.0 min 1.0 ml/min→3.0 min 3.0 ml/min→4.0 min 3.0 ml/min; UV-detection: 210 nm.

### Liquid Chromatography-Mass Spectroscopy (LC-MS): Method G

Instrument MS: Micromass ZQ; instrument HPLC: HP 1100 Series; UV DAD; column: Grom-Sil 120 ODS-4 HE 50 mm×2 mm, 3.0 μm; eluent A: water+500 μl 50% aqueous formic acid/l, eluent B: acetonitrile+500 μl 50% aqueous formic acid/l; gradient: 0.0 min 0% B→2.9 min 70% B→3.1 min 90% B→4.5 min 90% B; oven temp.: 50° C.; flow rate: 0.8 ml/min; UV-detection: 210 nm.

Preparative HPLC purifications are performed on a GROM-SIL 120 ODS-4 E 10 μm, 250 mm×30 mm column with acetonitrile/water gradients.

### Mass Determination

The mass determinations were carried out by MAT95 (Finnigan MAT).

Melting points are uncorrected.

¹H NMR spectra were recorded using either Bruker DRX-300 (300 MHz for ¹H) spectrometer or Brucker 500 UltraShieled™ (500 MHz for 1H). Chemical shifts are reported in parts per million (ppm) with tetramethylsilane (TMS) as an internal standard at zero ppm. Coupling constant (J) are given in hertz and the abbreviations s, d, t, q, m, and br refer to singlet, doblet, triplet, quartet, multiplet, and broad, respectively.

TLC was performed on a precoated silica gel plate (Merck silica gel 60 F-254). Silica gel (WAKO-gel C-200 (75-150 μm)) was used for all column chromatography separations. All chemicals were reagent grade and were purchased from Sigma-Aldrich, Wako pure chemical industries, Ltd., Great Britain, Tokyo kasei kogyo Co., Ltd., Nacalai tesque, Inc., Watanabe Chemical Ind. Ltd., Maybridge plc, Lancaster Synthesis Ltd., Merck KgaA, Germany, or Kanto Chemical Co., Ltd.

All starting materials are commercially available or can be prepared using methods cited in the literature.

### ASSAYS AND PHARMACOLOGICAL TESTS

The effect of the present compounds was examined by the following assays and pharmacological tests.

### [Measurement of Capsaicin-induced Ca²⁺ Influx in the Human VR1-transfected CHO Cell Line](Assay 1)

### (1) Establishment of the Human VR1-CHOluc9aeq Cell Line

Human vanilloid receptor (hVR1) cDNA was cloned from libraries of axotomized dorsal root ganglia (WO 00/29577). The cloned hVR1 cDNA was constructed with pcDNA3 vector and transfected into a CHOluc9aeq cell line. The cell line contains aequorin and CRE-luciferase reporter genes as read-out signals. The transfectants were cloned by limiting dilution in selection medium (DMEM/F12 medium (Gibco BRL) supplemented with 10% FCS, 1.4 mM Sodium pyruvate, 20 mM HEPES, 0.15% Sodium bicarbonate, 100 U/ml penicillin, 100 μg/ml streptomycin, 2 mM glutamine, non-essential amino acids and 2 mg/ml G418). Ca²⁺ influx was examined in the capsaicin-stimulated clones. A high responder clone was selected and used for further experiments in the project. The human VR1-CHOluc9aeq cells were maintained in the selection medium and passaged every 3-4 days at 1-2.5×10⁵ cells/flask (75 mm²).

### (2) Measurement of Ca²⁺ Influx Using FDSS-3000

Human VR1-CHOluc9aeq cells were suspended in a culture medium which is the same as the selection medium except for G418 and seeded at a density of 1,000 cells per well into 384-well plates (black walled clear-base/Nalge Nunc International). Following the culture for 48 hrs the medium was changed to 2 μM Fluo-3 AM (Molecular Probes) and 0.02% Puronic F-127 in assay buffer (Hank's balanced salt solution (HBSS), 17 mM HEPES (pH7.4), 1 mM Probenecid, 0.1% BSA) and the cells were incubated for 60 min at 25° C. After washing twice with assay buffer the cells were incubated with a test compound or vehicle for 20 min at 25° C. Mobilization of cytoplasmic Ca²⁺ was measured by FDSS-3000 (λₑₓ=488 nm, λₑₘ=540 nm/Hamamatsu Photonics) for 60 sec after the stimulation with 10 nM capsaicin. Integral R was calculated and compared with controls.

### [Measurement of the Capsaicin-induced Ca²⁺ Influx in Primary Cultured Rat Dorsal Root Ganglia Neurons] (Assay 2)

### (1) Preparation of Rat Dorsal Root Ganglia Neurons

New born Wister rats (5-11 days) were sacrificed and dorsal root ganglia (DRG) was removed. DRG was incubated with 0.1% trypsin (Gibco BRL) in PBS(−) (Gibco BRL) for 30 min at 37° C., then a half volume of fetal calf serum (FCS) was added and the cells were spun down. The DRG neuron cells were resuspended in Ham F12/5% FCS/5% horse serum (Gibco BRL) and dispersed by repeated pipetting and passing through 70 μm mesh (Falcon). The culture plate was incubated for 3 hours at 37° C. to remove contaminating Schwann cells. Non-adherent cells were recovered and further cultured in laminin-coated 384 well plates (Nunc) at 1×10⁴ cells/50 μl/well for 2 days in the presence of 50 ng/ml recombinant rat NGF (Sigma) and 50 μM 5-fluorodeoxyuridine (Sigma).

### (2) Ca²⁺ Mobilization Assay

DRG neuron cells were washed twice with HBSS supplemented with 17 mM HEPES (pH 7.4) and 0.1% BSA. After incubating with 2 μM fluo-3AM (Molecular Probe), 0.02% PF127 (Gibco BRL) and 1 mM probenecid (Sigma) for 40 min at 37° C., cells were washed 3 times. The cells were incubated with VR1 antagonists or vehicle (dimethylsulfoxide) and then with 1 μM capsaicin in FDSS-6000 (λₑₓ=480 nm, λₑₘ=520 nm/Hamamatsu Photonics). The fluorescence changes at 480 nm were monitored for 2.5 min. Integral R was calculated and compared with controls.

### [Organ Bath Assay to Measure the Capsaicin-induced Bladder Contraction] (Assay 3)

Male Wistar rats (10 week old) were anesthetized with ether and sacrificed by dislocating the necks. The whole urinary bladder was excised and placed in oxygenated Modified Krebs-Henseleit solution (pH 7.4) of the following composition (112 mM NaCl, 5.9 mM KCl, 1.2 mM MgCl₂, 1.2 mM NaH₂PO₄, 2 mM CaCl₂, 2.5 mM NaHCO₃, 12 mM glucose). Contractile responses of the urinary bladder were studied as described previously [Maggi C A et al: Br. J. Pharmacol. 108: 801-805, 1993]. Isometric tension was recorded under a load of 1 g using longitudinal strips of rat detrusor muscle. Bladder strips were equilibrated for 60 min before each stimulation. Contractile response to 80 mM KCl was determined at 15 min intervals until reproducible responses were obtained. The response to KCl was used as an internal standard to evaluate the maximal response to capsaicin. The effects of the compounds were investigated by incubating the strips with compounds for 30 min prior to the stimulation with 1 μM capsaicin (vehicle: 80% saline, 10% EtOH, and 10% Tween 80). One of the preparations made from the same animal was served as a control while the others were used for evaluating compounds. Ratio of each capsaicin-induced contraction to the internal standard (i.e. KCl-induced contraction) was calculated and the effects of the test compounds on the capsaicin-induced contraction were evaluated.

### [Measurement of Ca²⁺ Influx in the Human P2X1-transfected CHO Cell Line]

### (1) Preparation of the Human P2X1-transfected CHOluc9aeq Cell Line

Human P2X1-transfected CHOluc9aeq cell line was established and maintained in Dulbecco's modified Eagle's medium (DMEM/F12) supplemented with 7.5% FCS, 20 mM HEPES-KOH (pH 7.4), 1.4 mM sodium pyruvate, 100 U/ml penicillin, 100 μg/ml streptomycin, 2 mM glutamine (Gibco BRL) and 0.5 Units/ml apyrase (grade I, Sigma). The suspended cells were seeded in each well of 384-well optical bottom black plates (Nalge Nunc International) at 3×10³/50 μl/well. The cells were cultured for following 48 hrs to adhere to the plates.

### (2) Measurement of the Intracellular Ca²⁺ Levels

P2X1 receptor agonist-mediated increases in cytosolic Ca²⁺ levels were measured using a fluorescent Ca²⁺ chelating dye, Fluo-3 AM (Molecular Probes). The plate-attached cells were washed twice with washing buffer (HBSS, 17 mM HEPES-KOH (pH 7.4), 0.1% BSA and 0.5 units/ml apyrase), and incubated in 40 μl of loading buffer (1 μM Fluo-3 AM, 1 mM probenecid, 1 μM cyclosporin A, 0.01% pluronic (Molecular Probes) in washing buffer) for 1 hour in a dark place. The plates were washed twice with 40 μl washing buffer and 35 μl of washing buffer were added in each well with 5 μl of test compounds or 2′,3′-o-(2,4,6-trinitrophenyl) adenosine 5′-triphpsphate (Molecular Probes) as a reference. After further incubation for 10 minutes in dark 200 nM α, β-methylene ATP agonist was added to initiate the Ca²⁺ mobilization. Fluorescence intensity was measured by FDSS-6000 (λₑₓ=410 nm, λₑₘ=510 nm/Hamamatsu Photonics) at 250 msec intervals. Integral ratios were calculated from the data and compared with that of a control.

### [Measurement of Capsaicin-induced Bladder Contraction in Anesthetized Rats] (Assay 4)

### (1) Animals

Female Sprague-Dawley rats (200˜250 g/Charles River Japan) were used.

### (2) Catheter Implantation

Rats were anesthetized by intraperitoneal administration of urethane (Sigma) at 1.2 g/kg. The abdomen was opened through a midline incision, and a polyethylene catheter (BECTON DICKINSON, PE50) was implanted into the bladder through the dome. In parallel, the inguinal region was incised, and a polyethylene catheter (Hibiki, size 5) filled with 2 IU/ml of heparin (Novo Heparin, Aventis Pharma) in saline (Otsuka) was inserted into a common iliac artery.

### (3) Cystometric Investigation

The bladder catheter was connected via T-tube to a pressure transducer (Viggo-Spectramed Pte Ltd, DT-XXAD) and a microinjection pump (TERUMO). Saline was infused at room temperature into the bladder at a rate of 2.4 ml/hr. Intravesical pressure was recorded continuously on a chart pen recorder (Yokogawa). At least three reproducible micturition cycles, corresponding to a 20-minute period, were recorded before a test compound administration and used as baseline values.

### (4) Administration of Test Compounds and Stimulation of Bladder with Capsaicin

The saline infusion was stopped before administrating compounds. A testing compound dissolved in the mixture of ethanol, Tween 80 (ICN Biomedicals Inc.) and saline (1:1:8, v/v/v) was administered intraarterially at 10 mg/kg. 2 min after the administration of the compound 10 μg of capsaicin (Nacalai Tesque) dissolved in ethanol was administered intraarterially.

### (5) Analysis of Cystometry Parameters

Relative increases in the capsaicin-induced intravesical pressure were analyzed from the cystometry data. The capsaicin-induced bladder pressures were compared with the maximum bladder pressure during micturition without the capsaicin stimulation. The testing compounds-mediated inhibition of the increased bladder pressures was evaluated using Student's t-test. A probability level less than 5% was accepted as significant difference.

### [Measurement of Over Active Bladder in Anesthetized Cystitis Rats] (Assay 5)

### (1) Animals

Female Sprague-Dawley rats (180˜250 g/Charles River Japan) were used. Cyclophosphamide (CYP) dissolved in saline was administered intraperitoneally at 150 mg/kg 48 hours before experiment.

### (2) Catheter Implantation

Rats were anesthetized by intraperitoneal administration of urethane (Sigma) at 1.25 g/kg. The abdomen was opened through a midline incision, and a polyethylene catheter (BECTON DICKINSON, PE50) was implanted into the bladder through the dome. In parallel, the inguinal region was incised, and a polyethylene catheter (BECTON DICKINSON, PE50) filled with saline (Otsuka) was inserted into a femoral vein. After the bladder was emptied, the rats were left for 1 hour for recovery from the operation.

### (3) Cystometric Investigation

The bladder catheter was connected via T-tube to a pressure transducer (Viggo-Spectramed Pte Ltd, DT-XXAD) and a microinjection pump (TERUMO). Saline was infused at room temperature into the bladder at a rate of 3.6 ml/hr for 20 min. Intravesical pressure was recorded continuously on a chart pen recorder (Yokogawa). At least three reproducible micturition cycles, corresponding to a 20-minute period, were recorded before a test compound administration.

### (4) Administration of Test Compounds

A testing compound dissolved in the mixture of ethanol, Tween 80 (ICN Biomedicals Inc.) and saline (1:1:8, v/v/v) was administered intravenously at 0.05 mg/kg, 0.5 mg/kg or 5 mg/kg. 3 min after the administration of the compound, saline (Nacalai Tesque) was infused at room temperature into the bladder at a rate of 3.6 in/hr.

### (5) Analysis of Cystometry Parameters

The cystometry parameters were analyzed as described previously [Lecci A et al: Eur. J. Pharmacol. 259: 129-135, 1994]. The micturition frequency calculated from micturition interval and the bladder capacity calculated from a volume of infused saline until the first micturition were analyzed from the cystometry data. The testing compounds-mediated inhibition of the frequency and the testing compounds-mediated increase of bladder capacity were evaluated using unpaired Student's t-test. A probability levels less than 5% was accepted as significant difference. Data were analyzed as the mean±SEM from 4-7 rats.

### [Measurement of Acute Pain]

Acute pain is measured on a hot plate mainly in rats. Two variants of hot plate testing are used: In the classical variant animals are put on a hot surface (52 to 56° C.) and the latency time is measured until the animals show nociceptive behavior, such as stepping or foot licking. The other variant is an increasing temperature hot plate where the experimental animals are put on a surface of neutral temperature. Subsequently this surface is slowly but constantly heated until the animals begin to lick a hind paw. The temperature which is reached when hind paw licking begins is a measure for pain threshold.

Compounds are tested against a vehicle treated control group. Substance application is performed at different time points via different application routes (i.v., i.p., p.o., i.t., i.c.v., s.c., intradermal, transdermal) prior to pain testing.

### [Measurement of Persistent Pain]

Persistent pain is measured with the formalin or capsaicin test, mainly in rats. A solution of 1 to 5% formalin or 10 to 100 μg capsaicin is injected into one hind paw of the experimental animal. After formalin or capsaicin application the animals show nociceptive reactions like flinching, licking and biting of the affected paw. The number of nociceptive reactions within a time frame of up to 90 minutes is a measure for intensity of pain.

Compounds are tested against a vehicle treated control group. Substance application is performed at different time points via different application routes (i.v., i.p., p.o., i.t., i.c.v., s.c., intradermal, transdermal) prior to formalin or capsaicin administration.

### [Measurement of Neuropathic Pain]

Neuropathic pain is induced by different variants of unilateral sciatic nerve injury mainly in rats. The operation is performed under anesthesia. The first variant of sciatic nerve injury is produced by placing loosely constrictive ligatures around the common sciatic nerve (Bennett and Xie, Pain 33 (1988): 87-107). The second variant is the tight ligation of about the half of the diameter of the common sciatic nerve (Seltzer et al., Pain 43 (1990): 205-218). In the next variant, a group of models is used in which tight ligations or transections are made of either the L5 and L6 spinal nerves, or the L5 spinal nerve only (KIM S H; CHUNG J M, AN EXPERIMENTAL-MODEL FOR PERIPHERAL NEUROPATHY PRODUCED BY SEGMENTAL SPINAL NERVE LIGATION IN THE RA, PAIN 50 (3) (1992): 355-363). The fourth variant involves an axotomy of two of the three terminal branches of the sciatic nerve (tibial and common peroneal nerves) leaving the remaining sural nerve intact whereas the last variant comprises the axotomy of only the tibial branch leaving the sural and common nerves uninjured. Control animals are treated with a sham operation.

Postoperatively, the nerve injured animals develop a chronic mechanical allodynia, cold allodynia, as well as a thermal hyperalgesia. Mechanical allodynia is measured by means of a pressure transducer (electronic von Frey Anesthesiometer, IITC Inc.-Life Science Instruments, Woodland Hills, SA, USA; Electronic von Frey System, Somedic Sales AB, Hörby, Sweden). Thermal hyperalgesia is measured by means of a radiant heat source (Plantar Test, Ugo Basile, Comerio, Italy), or by means of a cold plate of 5 to 10° C. where the nocifensive reactions of the affected hind paw are counted as a measure of pain intensity. A further test for cold induced pain is the counting of nocifensive reactions, or duration of nocifensive responses after plantar administration of acetone to the affected hind limb. Chronic pain in general is assessed by registering the circadanian rhytms in activity (Sudo and Arndt, Universität zu Köln, Cologne, Germany), and by scoring differences in gait (foot print patterns; FOOTPRINTS program, Klapdor et al., 1997. A low cost method to analyse footprint patterns. J. Neurosci. Methods 75, 49-54).

Compounds are tested against sham operated and vehicle treated control groups. Substance application is performed at different time points via different application routes (i.v., i.p., p.o., i.t., i.c.v., s.c., intradermal, transdermal) prior to pain testing.

### [Measurement of Inflammatory Pain]

Inflammatory pain is induced mainly in rats by injection of 0.75 mg carrageenan or complete Freund's adjuvant into one hind paw. The animals develop an edema with mechanical allodynia as well as thermal hyperalgesia. Mechanical allodynia is measured by means of a pressure transducer (electronic von Frey Anesthesiometer, IITC Inc.-Life Science Instruments, Woodland Hills, SA, USA). Thermal hyperalgesia is measured by means of a radiant heat source (Plantar Test, Ugo Basile, Comerio, Italy, Paw thermal stimulator, G. Ozaki, University of California, USA). For edema measurement two methods are being used. In the first method, the animals are sacrificed and the affected hindpaws sectioned and weighed. The second method comprises differences in paw volume by measuring water displacement in a plethysmometer (Ugo Basile, Comerio, Italy).

Compounds are tested against uninflamed as well as vehicle treated control groups. Substance application is performed at different points via different application routes (i.v., i.p., p.o., i.t., i.c.v., s.c., intradermal, transdermal) prior to pain testing.

### [Measurement of Diabetic Neuropathic Pain]

Rats treated with a single intraperitoneal injection of 50 to 80 mg/kg streptozotocin develop a profound hyperglycemia and mechanical allodynia within 1 to 3 weeks. Mechanical allodynia is measured by means of a pressure transducer (electronic von Frey Anesthesiometer, IITC Inc.-Life Science Instruments, Woodland Hills, SA, USA).

Compounds are tested against diabetic and non-diabetic vehicle treated control groups. Substance application is performed at different time points via different application routes (i.v., i.p., p.o., i.t., i.c.v., s.c., intradermal, transdermal) prior to pain testing.

Results in capsaicin-induced Ca²⁺ influx assay in the human VR1-transfected CHO cell line (Assay 1) are shown in Examples and tables of the Examples below. For practical reasons, the compounds are grouped in four classes based on activity as follows:

IC₅₀=A (< or =) 0.1 μM<B (< or =) 0.5 μM<C (< or =) 1 μM<D

The compounds of the present invention also show excellent selectivity, and strong activity in other assays 2-5 and assays for pain described above.

### EXAMPLES

### Chapter I (Examples)

### Preparing Method of Starting Compounds

### 4-Amino-2,3-dihydro-1H-inden-2-yl acetate

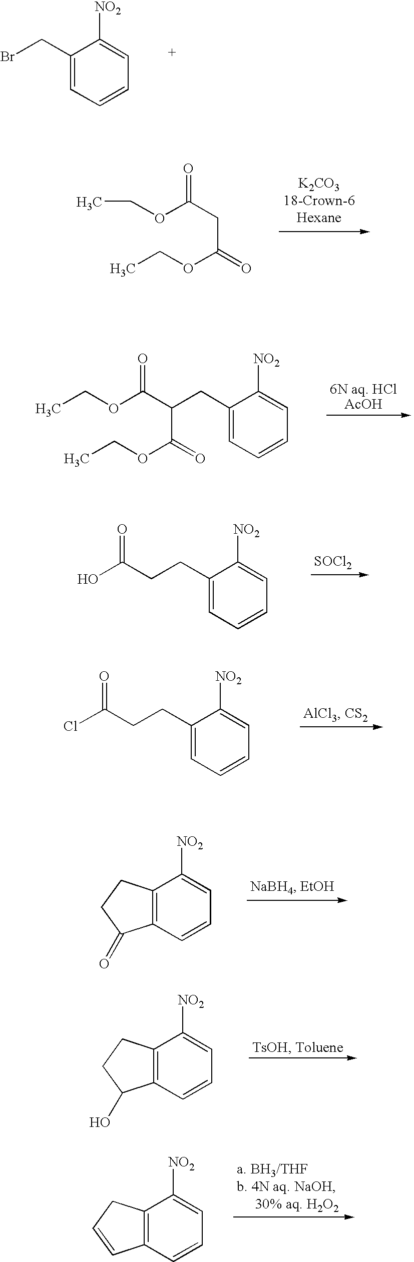

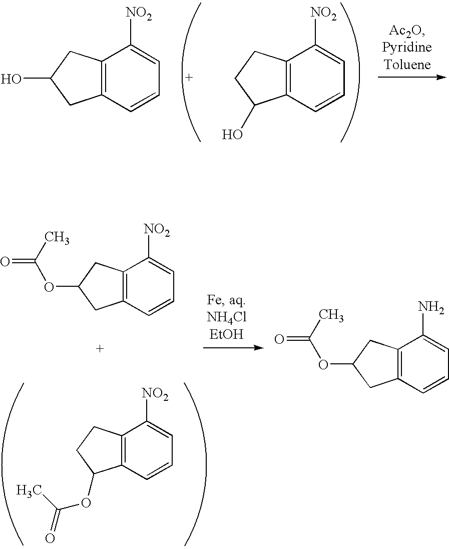

To a solution of 2-nitrobenzyl bromide (1.00 g, 4.63 mmol) and diethyl malonate (0.741 g, 4.63 mmol) in 30 ml of hexane was added potassium carbonate (0.640 g, 4.63 mmol) and 18-Crown-6 (0.012 g, 0.05 mmol). After stirred at 80° C. for 18 hours, the mixture was diluted with water and was extracted with ethyl acetate. The organic layer was washed with water, then with brine, and concentrated under reduced pressure to obtain crude diethyl (2-nitrobenzyl)malonate.

A solution of crude diethyl (2-nitrobenzyl)malonate in 6N aqueous HCl (15 ml) and acetic acid (15 ml) was stirred at refluxing temperature for 48 hours. After cooled to ambient temperature, the mixture was concentrated under reduced pressure. To the residue was added 10% aqueous NaOH solution and washed with ethyl acetate. The aqueous layer was acidified with aqueous HCl solution, and the mixture was extracted with ethyl acetate. The organic layer was dried over MgSO₄, filtered, and concentrated under reduced pressure to obtain 3-(2-nitrophenyl)propanoic acid.

¹H NMR (CDCl₃) δ 2.79 (t, J=7.6 Hz, 2H), 3.24 (t, J=7.6 Hz, 2H), 7.38-7.44 (m, 2H), 7.55 (dt, J=7.6, 1.6 Hz, 1H), 7.96 (dd, J=7.6, 1.6 Hz, 1H).

A solution of 3-(2-nitrophenyl)propanoic acid (1.20 g, 6.15 mmol) and thionyl chloride (0.878 g, 7.38 mmol) in dichloromethane (5 ml) was stirred and heated to reflux for 2 hours. The mixture was concentrated under reduced pressure to obtain 3-(2-nitrophenyl)propanoyl chloride. To the obtained crude 3-(2-nitrophenyl)propanoyl chloride (1.31 g, 6.15 mmol) was added CS₂, and aluminum trichloride (1.07 g, 8.0 mmol) was added portionwise at 0° C. The mixture was stirred at 70° C. for 3 hours, and after cooled to ambient temperature, water was added and extracted with ethyl acetate. The organic layer was dried over MgSO₄, filtered, and concentrated under reduced pressure. The obtained residue was purified by silica gel column chromatography (hexane:ethylacetate 10:1) to afford 4-nitroindan-1-one (0.44 g).

¹H NMR (CDCl₃) δ 2.79-2.82 (m, 2H), 3.64-3.66 (m, 2H), 7.62 (t, J=7.9 Hz, 1H), 8.09 (d, J=7.6 Hz, 1H), 8.47 (d, J=8.2 Hz, 1H).

To a solution of 4-nitroindan-1-one (0.381 g, 2.15 mmol) in ethanol (5 ml) was added sodium borohydride (0.048 g, 1.29 mmol) at 0° C., and the mixture was stirred at room temperature for 3 hours. Aqueous solution of ammonium chloride was added to the mixture, and extracted with ethyl acetate. The organic layer was dried over MgSO₄, filtered, and concentrated under reduced pressure to obtain 4-nitroindan-1-ol.

¹H NMR (CDCl₃) δ 1.90 (d, J=6.5 Hz, 1H), 2.00-2.07 (m, 1H), 2.56-2.63 (m, 1H), 3.25-3.33 (m, 1H), 3.54-3.60 (m, 1H), 5.30-5.35 (m, 1H), 7.44 (t, J=8.2 Hz, 1H), 7.72 (d, J=7.6 Hz, 1H), 8.12 (d, J=8.2 Hz, 1H).

A solution of 4-nitroindan-1-ol (0.385 g, 2.15 mmol) and p-toluenesulfonic acid (5.0 mg, 0.03 mmol) in toluene (30 ml) was stirred and heated to reflux for 16 hours. After cooled to ambient temperature, the mixture was washed with aqueous sodium bicarbonate solution. The organic layer was dried over MgSO₄, filtered, and concentrated under reduced pressure. The obtained residue was purified by preparatory TLC (hexane:ethylacetate 3:1) to afford 7-nitro-1H-indene (0.289 g).

¹H NMR (CDCl₃) δ 3.94 (s, 2H), 6.75 (dt, J=5.7, 1.9 Hz, 1H), 6.93 (dt, J=5.7, 1.6 Hz, 1H), 7.45 (t, J=8.2 Hz, 1H), 7.68 (d, J=7.6 Hz, 1H), 8.05 (d, J=8.2 Hz, 1H).

To a solution of 2,3-dimethyl-2-butene (21.5 mg, 0.31 mmol) in THF (2 ml) at 0° C. was added borane-THF (0.307 ml, 0.31 mmol) dropwise. After stirred for 1 hour at 0° C., 7-nitro-1H-indene (45.0 mg, 0.28 mmol) in THF (5 ml) was added dropwise, and the mixture was stirred for 2 hours at ambient temperature. The mixture was cooled to 0° C., and water (0.15 ml), 4N aqueous sodium hydroxide (0.45 ml), and 30% H₂O₂ (0.45 ml) were added. The mixture was then warmed to room temperature and poured into water, extracted with ethyl acetate and washed with brine. The organic layer was dried over MgSO₄, filtered, and concentrated under reduced pressure. To the obtained mixture in toluene (1 ml) was added acetic anhydride (40.8 mg, 0.40 mmol) and pyridine (0.4 ml), and then stirred for 16 hours at room temperature. The mixture was concentrated under reduced pressure, and the obtained residue was purified by preparatory TLC (hexane:ethylacetate 2:1) to obtain 4-nitro-2,3-dihydro-1H-inden-2-yl acetate (16.0 mg).

¹H NMR (CDCl₃) δ 2.03 (s, 3H), 3.12 (dd, J=17.5, 1.6 Hz, 1H), 3.40 (dd, J=17.5, 6.3 Hz, 1H), 3.60 (dd, J=19.2, 2.2 Hz, 1H), 3.74 (dd, J=19.2, 6.6 Hz, 1H), 5.58-5.62 (m, 1H), 7.39 (t, J=7.9 Hz, 1H), 7.54 (d, J=7.3 Hz, 1H), 8.06 (d, J=8.2 Hz, 1H).

To a mixture of 4-nitro-2,3-dihydro-1H-inden-2-yl acetate (100 mg, 0.45 mmol) and ammonium chloride (100 mg) in ethanol (6 ml) and water (3 ml) was added iron powder (300 mg) portionwise at room temperature. The mixture was stirred at 90° C. for 1 hour, and after cooled to room temperature, the mixture was diluted with ethylacetate. The mixture was filtered through a pad of celite, and the filtrate was washed with brine, dried over MgSO₄, filtered, and concentrated under reduced pressure to obtain 4-amino-2,3-dihydro-1H-inden-2-yl acetate.

¹H NMR (CDCl₃) δ 2.03 (s, 3H), 2.81 (dd, J=16.4, 2.8 Hz, 1H), 3.00 (dd, J=16.7, 2.8 Hz, 1H), 3.14 (dd, J=16.4, 6.6 Hz, 1H), 3.29 (dd, J=16.7, 6.6 Hz, 1H), 3.58 (br.s, 2H), 5.51-5.56 (m, 1H), 6.54 (d, J=7.9 Hz, 1H), 6.69 (d, J=7.3 Hz, 1H), 7.04 (t, J=7.9 Hz, 1H).

### Example 1-1

### 4-[({[4-Chloro-3-(trifluoromethyl)phenyl]amino}carbonyl)amino]-2,3-dihydro-1H-inden-2-yl acetate

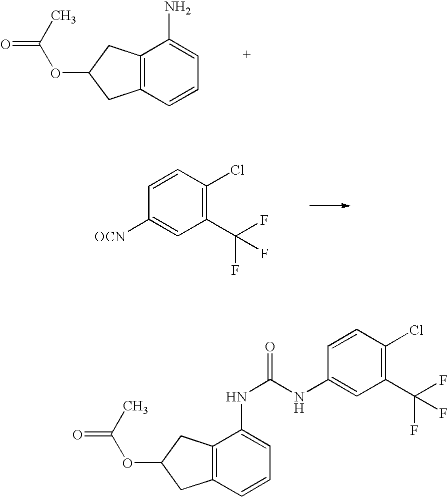

A mixture of 4-amino-2,3-dihydro-1H-inden-2-yl acetate (86.4 mg, 0.45 mmol) and 4-chloro-3-trifluoromethylphenyl isocyanate (110 mg, 0.50 mmol) in 1,4-dioxane (2 m) was stirred at 50° C. for 15 hours. The mixture was concentrated under reduced pressure, and to the obtained residue was added diisopropyl ether. The precipitate was collected to afford 4-[({[4-chloro-3-(trifluoromethyl)phenyl]amino}carbonyl)amino]-2,3-dihydro-1H-inden-2-yl acetate (128 mg).

¹H NMR (DMSO-d₆) δ 1.98 (s, 3H), 2.91 (ddd, J=19.6, 17.1, 1.9 Hz, 2H), 3.21-3.30 (m, 2H), 5.40-5.45 (m, 1H), 6.96 (d, J=7.3 Hz, 1H), 7.15 (t, J=7.9 Hz, 1H), 7.62 (s, 2H), 7.71 (d, J=8.2 Hz, 1H), 8.10 (s, 1H), 8.25 (s, 1H), 9.34 (s, 1H);

Molecular weight: 412.80

MS (M+H): 413

Mp 207-209° C.;

Activity class: C

In the similar manner as described in Example 1-1, compounds in Example 1-2 to 1-3 as shown in Table 1 were synthesized.

TABLE 1

| example | | | MS | | activity |
|---|---|---|---|---|---|
| # | structure | M.W. | (M + 1) | MP | class |
| 1-2 | 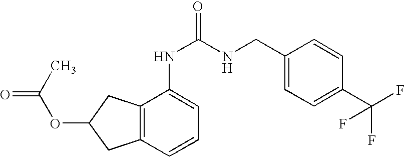 | 392,38 | 393 | 166-168 | A |
| 1-3 | 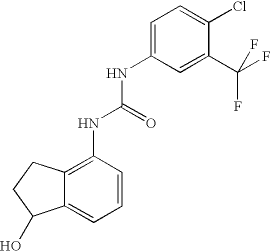 | 370,76 | 371 | 221-223 | A |

### Starting Material

### (6Ethoxy-5,8-dihydronaphthalen-1-yl)amine

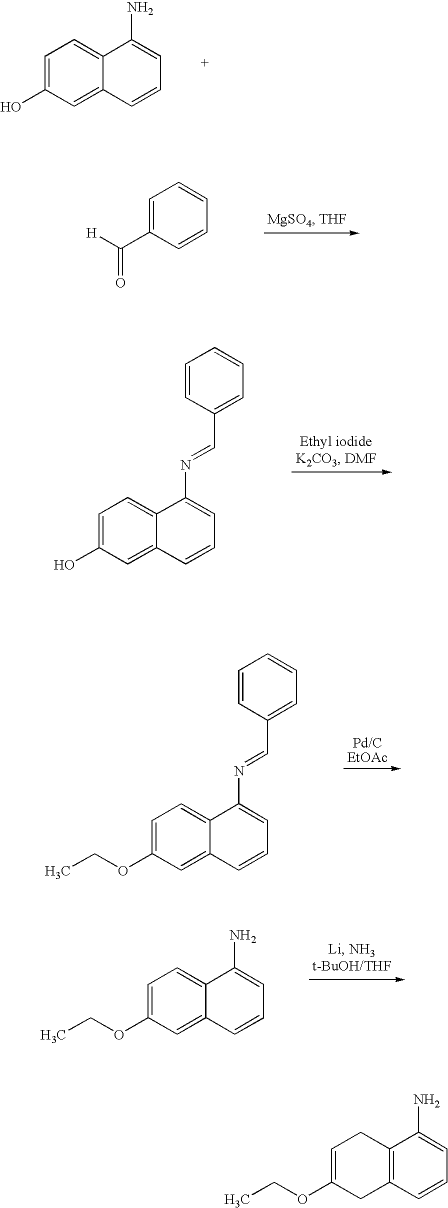

A mixture of 5-amino-2-naphthol (4.78 g, 30.0 mmol), benzaldehyde (3.50 g, 33.0 mmol), and magnesium sulfate (10.0 g) in THF (100 ml) was, heated to reflux for 16 hours. After cooled to ambient temperature, the mixture was filtered through a pad of celite and the filtrate was concentrated under reduced pressure. The obtained residue was recrystallized with diethylether to afford 5-{[phenylmethylene]amino}-2-naphthol (7.40 g).

¹H NMR (CDCl₃) δ 5.06 (br.s, 1H), 6.92 (d, J=6.6 Hz, 1H), 7.10-7.17 (m, 2H), 7.42-7.49 (dd, J=6.6 Hz, 1H), 7.45-7.55 (m, 4H), 8.00-8.02 (m, 2H), 8.27 (d, J=9.0 Hz, 1H), 8.56 (s, 1H)

Molecular weight: 247.30

MS (M+H): 248

To a solution of 5-{(phenylmethylene)amino}-2-naphthol (2.00 g, 8.09 mmol) in DMF (50 ml) was added ethyl iodide (1.39 g, 8.90 mmol) at room temperature and stirred at 50° C. for 2 hours. After cooled to ambient temperature, water was added and the mixture was extracted with ethyl acetate. The organic layer was washed with water and brine, dried over MgSO₄, filtered, and concentrated under reduced pressure. The obtained residue was purified by silica gel column chromatography (hexane:ethylacetate 15:1) to afford (6-ethoxy-1-naphthyl)(phenylmethylene)amine (1.54 g).

¹H NMR (CDCl₃) δ 1.49 (3H, t, J=6.8 Hz), 4.17 (2H, q, J=6.8 Hz), 6.91 (1H, dd, J=1.1, 7.5 Hz), 7.14-7.18 (2H, m), 7.41 (1H, dd, J=7.2, 7.2 Hz), 7.50-7.61 (4H, m), 7.99-8.03 (2H, m), 8.25 (1H, d, J=8.7 Hz), 8.55 (1H, s);

Molecular weight: 275.35

MS (M+H): 276

A mixture of (6-ethoxy-1-naphthyl)(phenylmethylene)amine (0.600 g, 2.18 mmol) and Pd/C (0.900 g) in ethyl acetate (15 ml) was stirred under argon at room temperature for 48 hours. The mixture was filtered through a pad of celite, and the filtrate was concentrated under reduced pressure. The obtained residue was purified by silica gel column chromatography (hexane:ethylacetate 4:1) to provide (6-ethoxy-1-naphthyl)amine (2.78 g).

¹H NMR (CDCl₃) δ 1.46 (3H, t, J=6.8 Hz), 4.06 (2H, brs), 4.13 (2H, q, J=6.8 Hz), 6.62 (1H, dd, J=1.5, 6.8 Hz), 7.08-7.12 (2H, m), 7.16-7.25 (2H, m), 7.70 (1H, d, J=9.8 Hz)

Molecular weight: 187.24

MS (M+H): 188

To a mixture of (6-ethoxy-1-naphthyl)amine (300 mg, 1.60 mmol) and tert-buthanol (641 mg, 8.65 mmol) in THF (4 ml) and liquid ammonia (55 ml) at −78° C. was added lithium (96.8 mg, 13.94 mmol) portionwise. After the mixture was stirred for 30 minutes at −78° C., methanol (9 ml) and water were added. Ammonia was removed at room temperature, and the resulted mixture was extracted with ethyl acetate. The organic layer was dried over MgSO₄, filtered, and concentrated under reduced pressure. The obtained residue was purified by silica gel column chromatography (hexane:ethylacetate 4:1) to afford (6-ethoxy-5,8-dihydronaphthalen-1-yl)amine (248 mg).

¹H NMR (CDCl₃) δ 1.33 (3H, t, J=6.8 Hz), 3.17 (1H, dd, J=3.4, 5.1 Hz), 3.20 (1H, dd, J=3.4, 5.1 Hz), 3.42 (1H, d, J=5.1 Hz), 3.43 (1H, d, J=5.1Hz), 3.57 (2H, brs), 3.81 (2H, q, J=6.8 Hz), 4.77 (1H, t, J=3.4 Hz), 6.52 (1H, d, J=7.9 Hz), 6.58 (1H, d, J=7.5 Hz), 6.98 (1H, dd, J=7.5, 7.9 Hz).

Molecular weight: 189.26

MS (M+H): 190

### Example 2-1

### N-[4Chloro-3-(trifluoromethyl)phenyl]-N′-(6-ethoxy-5,8-dihydronaphthalen-1-yl)urea

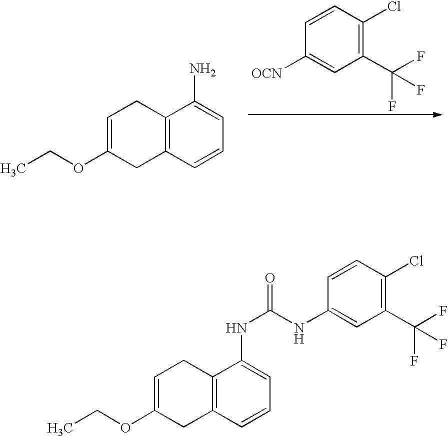

Next, to a solution of (6-ethoxy-5,8-dihydronaphthalen-1-yl)amine (108 mg, 0.57 mmol) in THF (5 ml) was added 4-chloro-3-trifluoromethyl isocyanate (139 mg, 0.63 mmol), and the mixture was stirred for 13 hours. Saturated aqueous solution of sodium carbonate was added and the mixture was extracted with ethylacetate. The organic layer was dried over MgSO₄, filtered, and concentrated under reduced pressure to obtain N-[4-chloro-3-(trifluoromethyl)phenyl]-N′-6-ethoxy-5,8-dihydronaphthalen-1-yl)urea (234 mg).

¹H NMR (DMSO-d₆) δ 1.26 (3H, t, J=6.8 Hz), 3.29-3.38 (4H, m), 3.80 (2H, q, J=6.8 Hz), 6.91 (1H, d, J=7.5 Hz), 7.14 (1H, dd, J=7.5, 7.9 Hz), 7.59-7.61 (2H, m), 8.01 (1H, s), 8.10 (1H, s), 9.45 (1H, s)

Molecular weight: 410.82

MS (M+H): 411

Mp 216° C.;

Activity class: B

### Example 2-2

### N-[4-Chloro-3-(trifluoromethyl)phenyl]-N′-(6-oxo-5,6,7,8-tetrahydronaphthalen-1-yl)urea

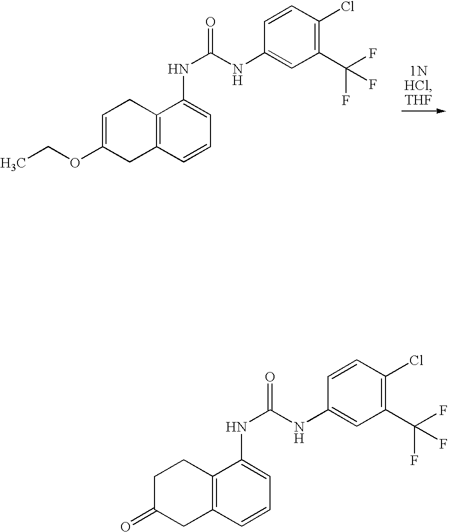

To a solution of N-[4-chloro-3-(trifluoromethyl)phenyl]-N′-6-ethoxy-5,8-dihydronaphthalen-1-yl)urea (50.0 mg, 0.12 mmol) was added aqueous 1N HCl solution at room temperature. After stirred for 20 minutes, saturated aqueous solution of sodium carbonate was added and the mixture was extracted with ethylacetate. The organic layer was dried over MgSO₄, filtered, and concentrated under reduced pressure. The obtained residue was purified by silica gel column chromatography (hexane:ethylacetate 1:2) to afford N-[4-chloro-3-trifluoromethyl)phenyl]-N′-(6-oxo-5,6,7,8-tetrahydronaphthalen-1-yl)urea (41.7 mg).

¹H NMR (Acetone-d₆) δ 2.44 (2H, t, J=6.4 Hz), 3.06 (2H, t, J=6.4 Hz), 3.57 (2H, s), 6.98 (1H, d, J=7.2 Hz), 7.19 (1H, dd, J=7.2, 7.5 Hz), 7.51-7.52 (2H, m), 7.74 (1H, dd, J=2.6, 8.7 Hz), 7.87 (1H, brs), 7.14 (1H, d, J=2.6 Hz), 8.69 (, 1H, brs);

Molecular weight: 382.77

MS (M+H): 383

Mp 219° C.;

Activity class: A

### Example 2-3

### N-[4-Chloro-3-(trifluoromethyl)phenyl]-N′-(6-hydroxy-5,6,7,8-tetrahydronaphthalen-1-yl)urea

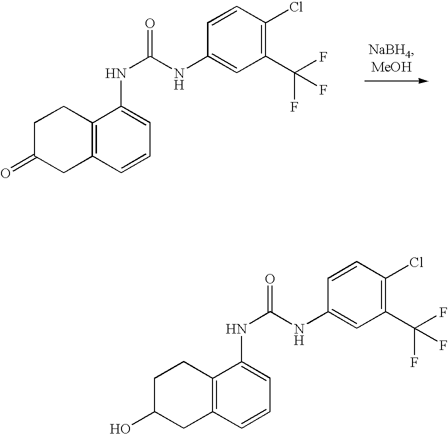

To a solution of N-[4-chloro-3-(trifluoromethyl)phenyl]-N′-6-oxo-5,6,7,8-tetrahydronaphthalen-1-yl)urea (70.0 mg, 0.18 mmol) in methanol (3 ml) was added sodium borohydride (7.61 mg, 0.20 mmol) at 0° C. After stirred for 30 minutes, the mixture was concentrated under reduced pressure and water was added. The mixture was extracted with ethylacetate, and the organic layer was dried over MgSO₄, filtered, and concentrated under reduced pressure to obtain N-[4-chloro-3-(trifluoromethyl)phenyl]-N′-(6-hydroxy-5,6,7,8-tetrahydronaphthalen-1-yl)urea (70.0 mg).

¹H NMR (Acetone-d₆) δ 1.75 (1H, m), 2.04 (1H, m), 2.59-3.04 (4H, m), 4.02 (1H, m), 6.84 (1H, d, J=7.2 Hz), 7.09 (1H, dd, J=7.2, 7.5 Hz), 7.50-7.53 (2H, m), 7.67 (1H, d, J=7.5 Hz), 7.72 (1H, dd, J=2.6, 8.7 Hz), 8.13 (1H, d, J=2.6 Hz), 8.77 (1H, s);

Molecular weight: 384.79

MS (M+H): 385

Mp 216° C.

Activity class: A

In the similar manner as described in Example 2-1, 2-2, or 2-3, compounds in Example 2-4 to 2-9 as shown in Table 2 were synthesized.

| example | | | MS | | activity |
|---|---|---|---|---|---|
| # | structure | M.W. | (M + 1) | MP | class |
| 2-4 | 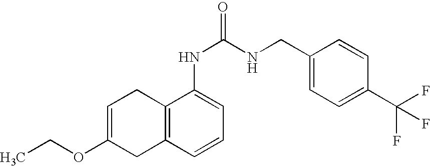 | 390,41 | 391 | 235 | C |
| 2-5 | 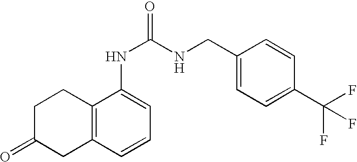 | 362,35 | 363 | 221 | A |
| 2-6 | 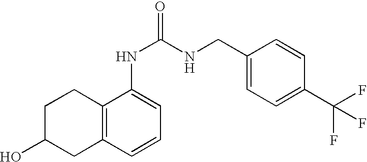 | 364,37 | 365 | 205 | A |
| 2-7 | 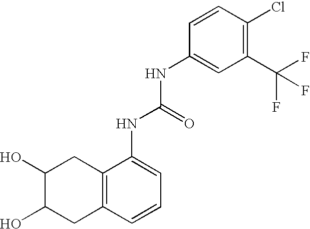 | 400,79 | 400 | 201 | A |
| 2-8 | 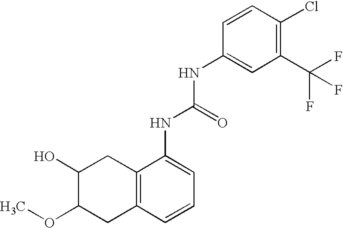 | 414,82 | 414 | 220-222 | B |
| 2-9 | 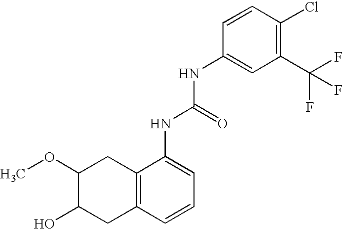 | 414,82 | 414 | 112-127 | A |

### Starting Material

### (7-Methyl-5,6,7,8-tetrahydronaphthalen-1-yl)amine

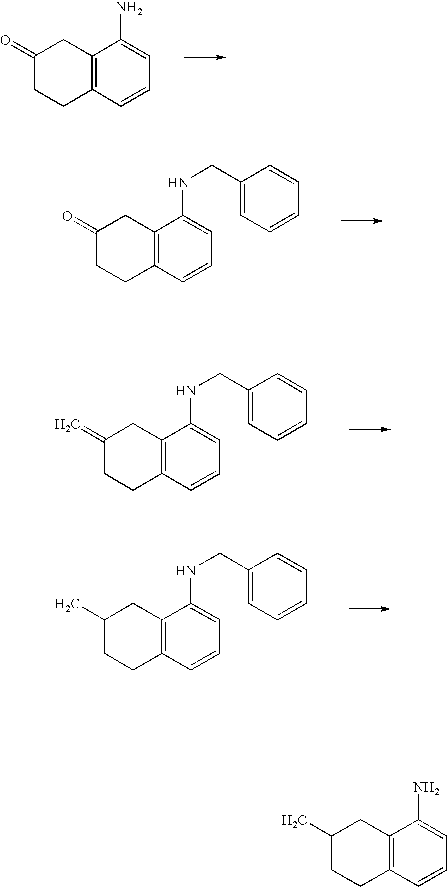

A mixture of 8-amino-3,4-dihydronaphthalen-2(1H)-one (1.61 g, 9.99 mmol), benzyl bromide (1.88 g, 11.0 mmol), and potassium carbonate (2.07 g, 15.0 mmol) in acetone (50 mL) was stirred at refluxing temperature for 16 hours. After the mixture was cooled to ambient temperature, it was filtered through a pad of Celite, and the filtrate was concentrated under reduced pressure. The obtained residue was purified by silica gel column chromatography (eluent: ethylacetate/hexane=1/10) to provide 8-(benzylamino)-3,4-dihydronaphthalen-2(1H)-one (1.87 g).

¹H NMR (CDCl₃) δ 2.65 (dd, J=12.9 Hz, 6.6 Hz, 2H), 3.10 (dd, J=12.9 Hz, 6.6 Hz, 2H), 3.28 (s, 2H), 3.71 (brs, 1H), 4.31 (s, 2H), 6.55 (d, J=8.1 Hz, 1H), 6.60 (d, J=8.1 Hz, 1H), 7.12 (t, J=8.1 Hz, 1H), 7.23-7.40 (m, 5H);

Molecular weight: 251.33

MS (M+H): 252

To a suspension of methyltriphenylphosphonium iodide (2.12 g, 5.25 mmol) in tetrahydrofuran (100 ml) was added sodium tert-butoxide (0.56 g, 5.83 mmol) at 0° C. After the mixture was stirred for 30 minutes, a solution of 8-(benzylamino)-3,4-dihydronaphthalen-2(1H)-one (0.66 g, 2.63 mmol) in tetrahydrofuran (10 ml) was added at room temperature and then stirred at 100° C. for 13 hours. The mixture was cooled to ambient temperature and was poured into water. The mixture was extracted with ethylacetate, and the organic layer was dried over Na₂SO₄, filtered, and concentrated under reduced pressure to obtain N-benzyl-7-methylene-5,6,7,8-tetrahydronaphthalen-1-amine (0.367 g).

¹H NMR (CDCl₃) δ 2.45 (dd, J=12.9 Hz, 6.6 Hz, 2H), 2.86 (dd, J=12.9 Hz, 6.6 Hz, 2H), 3.20 (s, 2H), 3.80 (brs, 1H), 4.37 (s, 2H), 4.86-4.90 (m, 2H), 6.50 (d, J=8.1 Hz, 1H), 6.55 (d, J=8.1 Hz, 1H), 7.05 (t, J=8.1 Hz, 1H), 7.29-7.41 (m, 5H);

Molecular weight: 249.36

MS (M+H): 250

To a solution of N-benzyl-7-methylene-5,6,7,8-tetrahydronaphthalen-1-amine (0.50 g, 2.00 mmol) in tetrahydro furan (5 ml) was added 0.5 M tetrahydrofuran solution of 9-borabicyclo[3.3.1]nonane dimer (8.20 ml, 4.10 mmol) at 0° C. and then stirred at room temperature for 8 hours. To the resulting mixture was added 3N aqueous solution of sodium hydroxide 82 ml) followed by aqueous 33% hydrogen peroxide solution (2 ml), and the mixture was stirred at room temperature for 6 hours. The mixture was extracted with ethylacetate, and the organic layer was washed with brine, dried over Na₂SO₄, filtered, and concentrated under reduced pressure. The obtained residue was purified by silica gel column chromatography (eluent: ethylacetate/hexane=1/4) to provide N-benzyl-7-methyl-5,6,7,8-tetrahydronaphthalen-1-amine (0.069 g).

¹H NMR (CDCl₃) δ 1.11 (d, J=6.9 Hz, 3H), 1.22-1.24 (m, 1H), 1.55-1.65 (m, 1H), 1.80-2.05 (m, 2H), 2.74-2.76 (m, 2H), 4.36 (s, 2H), 4.45 (brs, 1H), 5.50 (brs, 1H), 6.49 (m, 2H), 7.05 (t, J=9.0 Hz, 1H), 7.28-7.40 (m, 5H).

A mixture of N-benzyl-7-methyl-5,6,7,8-tetrahydronaphthalen-1-amine (90.0 mg, 0.346 mmol) and palladium carbon (10.0 mg) in ethylacetate (10 ml) was stirred under hydrogen for 1 hour. The mixture was filtered through a pad of celite, and the filtrate was concentrated under reduced pressure. The obtained residue was purified by column chromatography (eluent: ethylacetate/hexane=1/3) to provide (7-methyl-5,6,7,8-tetrahydronaphthalen-1-yl)amine (46.0 mg).

¹H NMR (CDCl₃) δ 1.05 (d, J=6.0 Hz, 3H), 1.09-1.19 (m, 1H), 1.46-1.84 (m, 3H), 2.63-2.69 (m, 2H), 4.25 (brs, 2H, 4.36 (brs, 1H), 6.45-6.49 (m, 2H), 6.93 (t, J=6.0 Hz, 1H);

Molecular weight: 161.25

MS (M+H): 162

### Example 3-1

### N-[4-Chloro-3-(trifluoromethyl)phenyl]-N′-(7-methyl-5,6,7,8-tetrahydronaphthalen-1-yl)urea

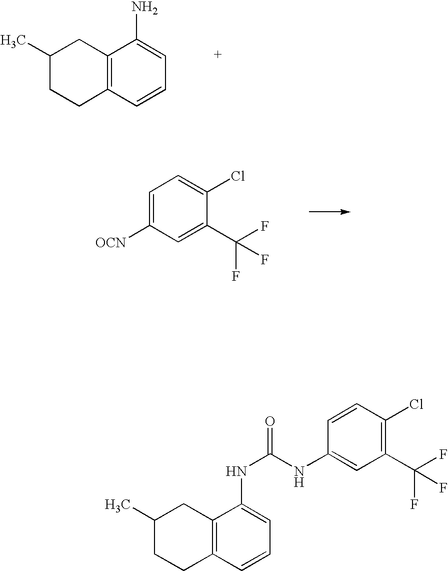

A mixture of (7-methyl-5,6,7,8-tetrahydronaphthalen-1-yl)amine (30.0 mg, 0.186 mmol) and 4-chloro-3-trifluoromethyl isocyanate (50.0 mg, 0.220 mmol) in tetrahydrofuran (10 ml) was stirred at room temperature for 16 hours. After the mixture was concentrated under reduced pressure, the obtained residue was purified by silica gel column chromatography (eluent: ethylacetate/hexane=1/3) to provide N-[4-chloro-3-trifluoromethyl)phenyl]-N′-(7-methyl-5,6,7,8-tetrahydronaphthalen-1-yl)urea (42.0 mg).

¹H NMR (MeOD-d₃) δ 1.10 (d, J=6.0 Hz, 3H), 1.35-1.67 (m, 1H), 1.70-1.92 (m, 1H), 1.93-2.15 (m, 3H), 2.61-2.70 (m, 2H), 3.88-3.92 (m, 1H), 4.39 (d, J=6.0 Hz, 1H), 6.86 (d, J=9.0 Hz, 1H), 7.15 (d, J=9.0 Hz, 1H), 7.48 (d, J=9.0 Hz, 1H), 7.59-7.66 (m, 2H), 8.00 (s, 1H).

Molecular weight: 382.82

MS (M+H): 383

Activity Class: A

### Starting Material

### (8-Amino-1,2,3,4-tetrahydronaphthalen-2-yl)methanol

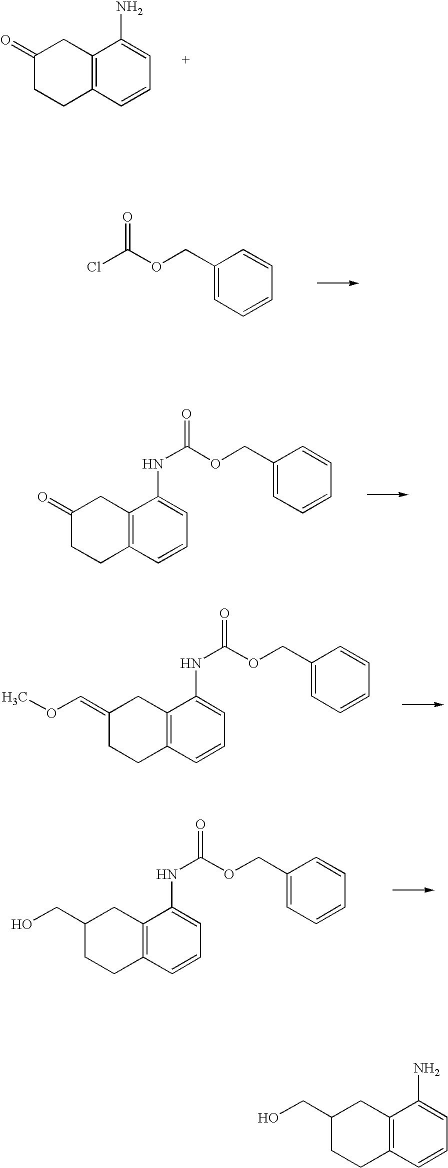

To a solution of 8-amino-3,4-dihydronaphthalen-2(1H)-one (5.00 g, 31.0 mmol) and pyridine (3.68 g, 46.5 mmol) in tetrahydrofuran (60 ml) was added benzyl chloroformate (6.35 g, 37.2 mmol) at 0° C. After the mixture was stirred at room temperature for 1 hour, it was poured into water and extracted with ethylacetate. The organic layer was washed with brine, dried over MgSO₄, filtered, and concentrated under reduced pressure. The obtained residue was washed with diethylether to provide benzyl (7-oxo-5,6,7,8-tetrahydronaphthalen-1-yl)carbamate (6.52 g).

¹H NMR (CDCl₃) δ 2.58 (t, J=6.8 Hz, 2H), 3.08 (t, J=6.8 Hz, 2H), 3.47 (s, 2H), 5.19 (s, 2H), 6.37 (brs, 1H), 7.07 (d, J=7.3 Hz, 1H), 7.22 (t, J=7.9 Hz, 1H), 7.33-7.50 (m, 6H).

To 2.6 M solution of n-butyllithium in hexane (1.72 ml) cooled at 0° C. was added diisopropylamine (452 mg, 4.47 mmol) dropwise. After the mixture was stirred at room temperature for 15 minutes, a solution of benzyl (7-oxo-5,6,7,8-tetrahydronaphthalen-1-yl)carbamate (600 mg, 2.03 mmol) in tetrahydrofuran (1 ml) at −78° C. and stirred for 1 hour. A solution of methoxymethyl(diphenyl)phosphine (550 mg, 2.23 mmol) in tetrahydrofuran (1 mL) was added to the reaction mixture at −78° C. and then stirred for 16 hours at room temperature. The resulting mixture was poured into water and extracted with ethylacetate. The organic layer was washed with brine, dried over MgSO₄, filtered, and concentrated under reduced pressure. The obtained residue was purified by silica gel column chromatography (eluent: hexane/ethylacetate=10/1) to provide benzyl [(7E)-7-(methoxymethylene)-5,6,7,8-tetrahydronaphthalen-1-yl]carbamate (109 mg).

¹H NMR (CDCl₃) δ 2.25 (t, J=6.3 Hz, 2H), 2.79 (t, J=6.3 Hz, 2H), 3.33 (s, 2H), 3.60 (s, 3H), 5.21 (s, 2H), 5.95 (s, 1H), 6.49 (brs, 1H), 6.85 (d, J=7.6 Hz, 1H), 7.12 (t, J=7.9 Hz, 1H), 7.33-7.43 (m, 5H), 7.73 (brs, 1H).

A solution of benzyl [(7E)-7-methoxymethylene)-5,6,7,8-tetrahydronaphthalen-1-yl]carbamate (51.0 mg, 0.16 mmol) in a mixture of tetrahydrofuran (3 ml) and 2N aqueous HCl (6 ml) was stirred at room temperature for 2 hours, and then extracted with ethylacetate. The organic layer was washed with brine, dried over MgSO₄, filtered, and concentrated under reduced pressure. The obtained residue was dissolved in ethanol (2 ml) and sodium borohydride (5.97 mg, 0.16 mmol) was added at room temperature. After stirred for 2 hours, the mixture was poured into water and extracted with diethylether. The organic layer was washed with brine, dried over MgSO₄, filtered, and concentrated under reduced pressure. The obtained residue was purified by silica gel column chromatography (eluent: hexane/ethylacetate=4/1) to provide benzyl [7-(hydroxymethyl)-5,6,7,8-tetrahydronaphthalen-1-yl]carbamate (34.0 mg).

¹H NMR (CDCl₃) δ 1.39 (m, 1H), 1.48 (brs, 1H), 1.95-1.98 (m, 2H), 2.24 (dd, J=16.1 Hz, 10.1 Hz, 1H), 2.72 (dd, J=16.1 Hz, 5.2 Hz, 1H), 2.77-2.88 (m, 3H), 3.63-3.65 (m, 2H), 5.20 (s, 2H), 6.90 (d, J=7.6 Hz, 1H), 7.13 (d, J=7.7 Hz, 1H), 7.32-7.42 (m, 5H), 7.62 (brs, 1H).

A mixture of benzyl [7-(hydroxymethyl)-5,6,7,8-tetrahydronaphthalen-1-yl]carbamate (32.0 mg, 0.10 mmol) and palladium carbon (30 mg) in ethanol (2 ml) was stirred under hydrogen at room temperature for 16 hours. The resulting mixture was filtered through a pad of celite, and the filtrate was concentrated under reduced pressure. The obtained residue was purified by silica gel column chromatography (eluent: hexane/ethylacetate=4/1) to provide (8-amino-1,2,3,4-tetrahydronaphthalen-2-yl)methanol (11.0 mg).

¹H NMR (CDCl₃) δ 1.37-1.44 (m, 2H), 1.94-2.04 (m, 2H), 2.14 (dd, J=16.1 Hz, 10.4 Hz, 1H), 2.64 (dd, J=15.7 Hz, 15.3 Hz, 1H), 2.79-2.82 (m, 2H), 3.58 (brs, 2H), 3.69 (d, J=15.3 Hz, 2H), 6.53 (d, J=7.9 Hz, 1H), 6.57 (d, J=7.5 Hz, 1H), 6.95 (t, J=15.3 Hz, 1H).

### Example 4-1

### N-[4-Chloro-3-(trifluoromethyl)phenyl]-N′-[7-(hydroxymethyl)-5,6,7,8-tetrahydronaphthalen-1-yl]urea

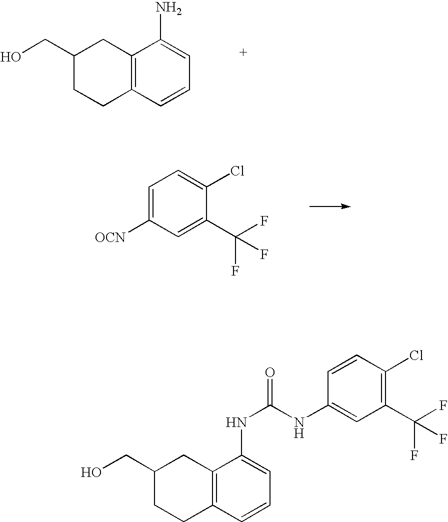

A mixture of (8-amino-1,2,3,4-tetrahydronaphthalen-2-yl)methanol (11.0 mg, 0.06 mmol) and 4-chloro-3-trifluoromethylphenyl isocyanate (13.7 mg, 0.06 mmol) in 1,4-dioxane (2 ml) was stirred for 2 hours at 50° C. The resulting mixture was concentrated under reduced pressure, and the obtained residue was washed with diisopropyl ether to provide N-[4-chloro-3-(trifluoromethyl)phenyl]-N′-[7-(hydroxymethyl)-5,6,7,8-tetrahydronaphthalen-1-yl]urea (14.0 mg).

¹H NMR (DMSO-d₆) δ 1.31 (m, 1H), 1.75-1.83 (m, 1H), 1.85-1.91 (m, 1H), 2.21 (dd, J=16.4 Hz, 10.4 Hz, 1H), 2.70-2.81 (m, 3H), 3.44 (t, J=5.7 Hz, 1H), 4.67 (t, J=5.1 Hz, 1H), 6.83 (d, J=7.5 Hz, 1H), 7.05 (t, J=7.8 Hz, 1H), 7.56-7.64 (m, 3H), 7.94 (s, 1H), 8.10 (d, J=2.2 Hz, 1H), 9.49 (s, 1H).

mp 194-196° C.;

Molecular weight: 398.81

MS (M+H): 399

Activity Class: A

### Chapter II (Examples)

### Preparing Method of Starting Compounds

### [Starting Compound A]

7-ethoxy-5,8-dihydronaphthalen-1-ylamine

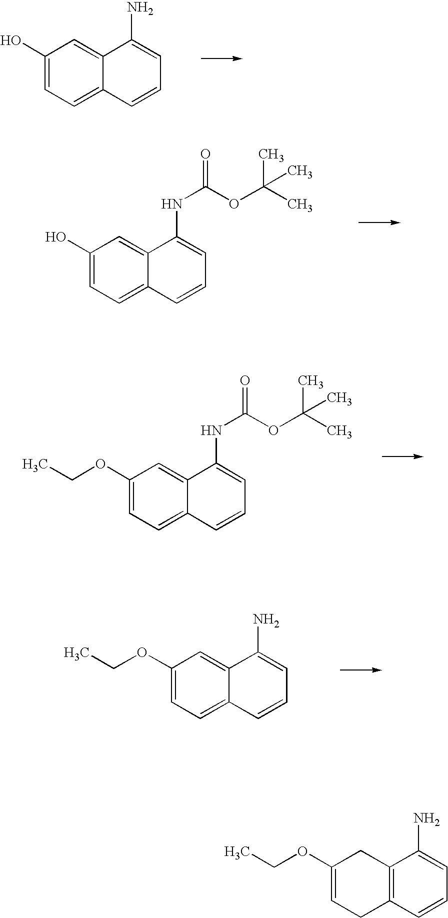

To a stirred solution of 8-amino-2-naphthol (50.0 g, 314 mmol) in tetrahydrofuran (1000 mL) was added di-t-butyldicarbonate (68.6 g, 314 mmol). The mixture was stirred at 70° C. for 18 hours. After the mixture was cooled to room temperature, solvent was removed under reduced pressure.

To the residue was added ethylacetate, and washed with saturated aqueous solution of sodium carbonate and then with water. The extracted organic layer was dried over Na₂SO₄, filtered, and concentrated under reduced pressure. To the obtained residue was added diisopropyl ether, and the precipitate was filtered and dried to afford N-t-butoxycarbonyl-8-amino-2-naphthol (64.2 g, 79% yield).

Next, to a mixture of N-t-butoxycarbonyl-8-amino-2-naphthol (64.0 g, 247 mmol) and Cesium carbonate (161 g, 493 mmol) in 300 mL anhydrous DMF was added iodoethane (42.3 g, 272 mmol) at room temperature. The mixture was stirred at 60° C. for 2 hours. Water was added to the-mixture, and the product was extracted with ethylacetate. The organic layer was washed with water and brine, dried over Na₂SO₄, filtered, and concentrated under reduced pressure. To the obtained residue was added diisopropyl ether and the precipitate was collected and dried to afford (7-ethoxy-naphthalen-1-yl)-carbamic acid t-butyl ester (47.9 g, 67.5% yield).

Next, to a (7-ethoxy-naphthalen-1-yl)-carbamic acid t-butyl ester (47.9 g, 167 mmol) in 100 mL anhydrous 1,4-dioxane was added 4N HCl in 1,4-dioxane (100 mL) at 0° C. The mixture was stirred at room temperature for 2 hours. Diisopropyl ether was added to the reaction mixture and the precipitate was filtered. To the obtained solid was added saturated sodium bicarbonate and the product was extracted with ethylacetate. The organic layer was dried over Na₂SO₄, filtered, and concentrated under reduced pressure to afford 7-ethoxy-naphthalen-1-ylamine (27.0 g, 86.3% yield).

Next, to a flask containing a mixture of 7-ethoxy-naphthalen-1-ylamine (1.80 g, 9.61 mmol) and t-buthanol (2.13 g, 28.8 mmol) in tetrahydrofuran (20 mL) was collected liquid ammonia (300 mL) at −78° C. To the mixture was added lithium (0.200 g, 28.8 mmol) over 30 minutes and stirred at −78° C. for 1 hour. Methanol and water was added, and the mixture was stirred at room temperature for 16 hours to allow ammonia to evaporate. To the obtained residue was added ethylacetate. The organic layer was washed with water, dried over Na₂SO₄, filtered, and concentrated under reduced pressure to afford 7-ethoxy-5,8-dihydronaphthalen-1-ylamine (1.37 g, 76% yield).

### [Starting Compound B]

8-amino-1,2,3,4-tetrahydro-naphthalen-2-ol

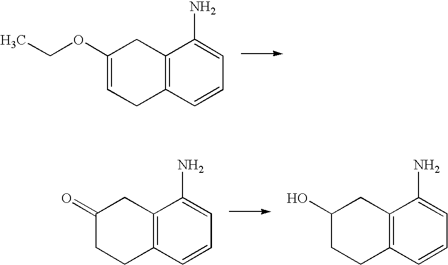

To a stirred solution of 7-ethoxy-5,8-dihydronaphthalen-1-ylamine (1.07 g, 5.65 mmol) in tetrahydrofuran (30 mL) was added solution of aqueous 2N HCl (10 mL), and stirred at 40° C. for 1 hour. The mixture was neutralized with addition of sodium bicarbonate, and the product was extracted with ethylacetate. The organic layer was washed with water, dried over Na₂SO₄, filtered, and concentrated under reduced pressure to afford 8-amino-3,4-dihydro-1H-naphthalen-2-one (0.71 g, 78% yield).

Next, to 8-amino-3,4-dihydro-1H-naphthalen-2-one (0.050 g, 0.318 mmol) in methanol (10 mL) was added sodium borohydride (0.030 g, 0.175 mmol) at 0° C., and the mixture was stirred for 1 hour. The mixture was poured into water, and the product was extracted with ethylacetate. The organic layer was dried over Na₂SO₄, filtered, and concentrated under reduced pressure to afford 8-amino-1,2,3,4-tetrahydro-naphthalen-2-ol (0.037 g, 71% yield).

### [Starting Compound C]

### 8-Amino-1,2,3,4-tetrahydro-naphthalen-2-ol (enantiomer)

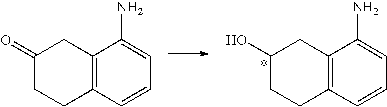

To a stirred solution of benzeneruthenium(II) chloride dimer (3.10 mg, 0.006 mmol) and (1S, 2R)-(−)-cis-1-amino-2-indanol (3.7 mg, 0.025 mmol) in degaussed isopropanol was heated at 80° C. for 20 minutes under argon. The mixture was added to the solution of 8-amino-3,4-dihydro-1H-naphthalen-2-one (50 mg, 0.310 mmol) in isopropanol (3 mL) at room temperature. A solution of potassium hydroxide (3.48 mg, 0.062 mmol) in isopropanol (1 mL) was added, and the mixture was stirred at 45° C. for 1 hour. The mixture was passed through silica gel and washed with ethylacetate. The filtrate was concentrated under reduced pressure to afford 8-amino-1,2,3,4-tetrahydro-naphthalen-2-ol enantiomer (33.0 mg, 65% yield).

The other enantiomer of 8-amino-1,2,3,4-tetrahydronaphthalen-2-ol was obtained in the same fashion replacing (1S,2R)-(−)-cis-1-amino-2-indanol with (1R,2S)-(+)-cis-1-amino-2-indanol.

### [Starting Compound D]

(7-hydroxy-5,6,7,8-tetrahydro-naphthalen-1-yl)-carbamic acid phenyl ester

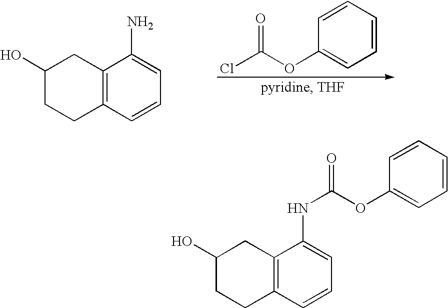

To a stirred solution of 8-amino-1,2,3,4-tetrahydro-naphthalen-2-ol (30.0 mg, 0.18 mmol) and pyridine (21.8 mg, 0.28 mmol) in 1.0 mL THF was added phenyl chloroformate (30.2 mg, 0.19 mmol), and the mixture was stirred for 1 hour at room temperature. To the product mixture was added water and extracted with ethylacetate. The organic layer was washed with brine, dried over Na₂SO₄, filtered and concentrated under reduced pressure. The obtained residue was triturated with ethylacetate and hexane to afford (7-hydroxy-5,6,7,8-tetrahydro-naphthalen-1-yl)-carbamic acid phenyl ester (25.2 mg, 48% yield).

### Example 1-1

### N-1,3-benzodioxol-5-yl-N′-(7-hydroxy-5,6,7,8-tetrahydronaphthalen-1-yl)urea

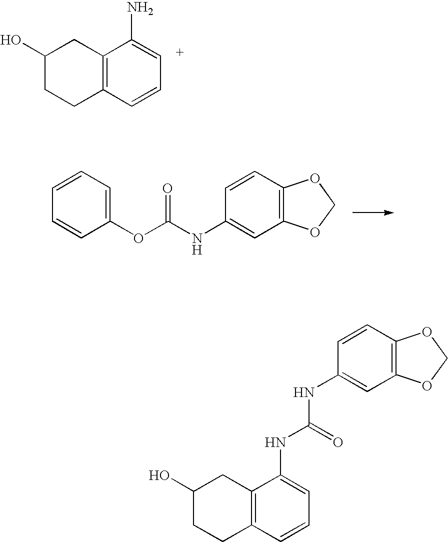

To a solution of phenyl 1,3-benzodioxol-5-ylcarbamate (51.5 mg, 0.20 mmol) in dimethylsulfoxide (1 mL) was added 8-amino-1,2,3,4-tetrahydronaphthalen-2-ol (32.6 mg, 0.20 mmol) at room temperature. The mixture was stirred at 100° C. for 1.5 hours, then the mixture was concentrated under reduced pressure. The resulting residue was purified by preparatory TLC (hexane/ethylacetate=1/1) to obtain N-1,3-benzodioxol-5-yl-N′-(7-hydroxy-5,6,7,8-tetrahydronaphthalen-1-yl)urea (7.10 mg).

¹H NMR (DMSO-d₆) δ 1.55-1.66 (m, 1H), 1.82-1.94 (m, 1H), 2.38 (dd, J=16.8, 8.1 Hz, 1H), 2.79-2.91 (m, 3H), 3.89-3.99 (m, 1H), 4.88 (d, J=4.2 Hz, 1H), 5.96 (s, 2H), 6.73 (dd, J=2.1, 8.4 Hz, 1H), 6.77 (d, J=7.8 Hz, 1H), 6.83 (d, J=8.4 Hz, 1H), 7.03 (t, J=8.1 Hz, 1H), 7.22 (d, J=2.1 Hz, 1H), 7.64 (d, J=7.8 Hz, 1H), 7.72 (s, 1H), 8.93 (s, 1H);

Molecular weight: 326.36

MS (M+H): 327

Mp 209-211° C.;

Activity grade: C

In the similar manner as described in Example 1-1, compounds in Example 1-2 to 1-13 as shown in Table 1 were synthesized.

TABLE 1

| example | | | MS | | activity |
|---|---|---|---|---|---|
| # | structure | M.W. | (M + 1) | MP | class |
| 1-2 | 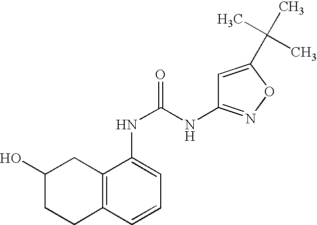 | 329,40 | 330 | amorphous | B |
| 1-3 | 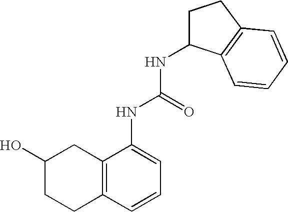 | 322,41 | 323 | 193-195 | B |
| 1-4 | 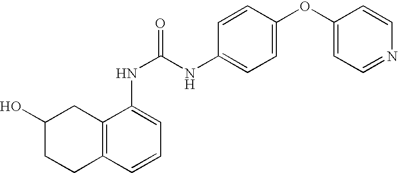 | 375,43 | 376 | 200-202 | B |
| 1-5 | 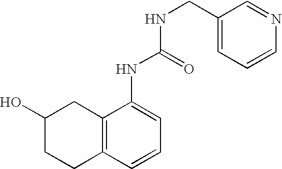 | 297,36 | 298 | 203 | C |
| 1-6 | 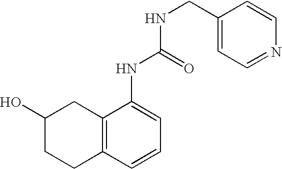 | 297,36 | 298 | 243 | C |
| 1-7 | 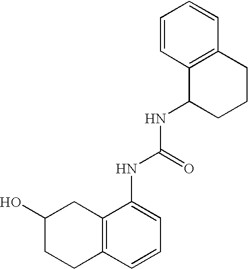 | 336,44 | 337 | 216 | A |
| 1-8 | 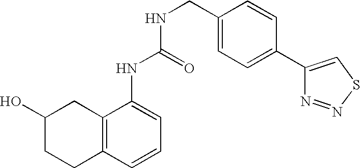 | 380,47 | 381 | 225 | A |
| 1-9 | 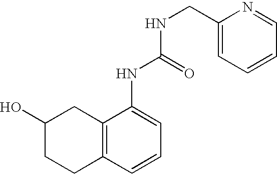 | 297,36 | 298 | 230 | C |
| 1-10 | 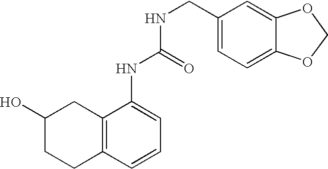 | 340,38 | 341 | 190 | A |
| 1-11 | 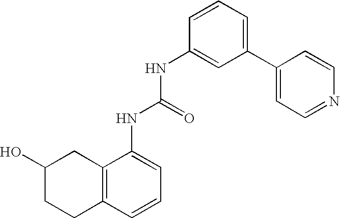 | 359,43 | 360 | 208 | A |
| 1-12 | 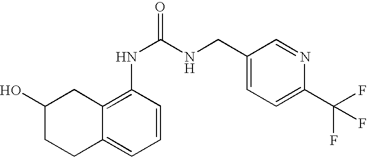 | 365,35 | 366 | 211-212 | A |
| 1-13 | 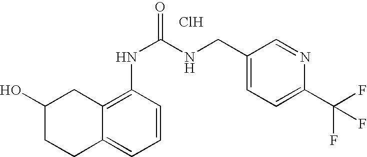 | 401,81 | 366 | 204-205 | A |

### Starting Material

1-[2,2-difluoro-1,3-benzodioxol-5-yl]methanamine

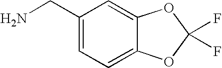

2,2-Difluoro-1,3-benzodioxole-5-carbontrile (1000 mg, 5.46 mol) in ethanol (100 ml) is treated in the presence of Pd/C (200 mg) under a hydrogen atmosphere of 3 bar for 1 h. The catalyst is filtered off. The solvent is removed under reduced pressure and the crude mixture is treated with diethyl ether. The resulting crystals are separated from the solvent via a glass filter.

Yield: 650 mg (64%)

¹H NMR (300 MHz, DMSO-d₆) δ 3.79 (s, 2H), 7.19 (d, 1H), 7.35 (d, 1H), 7.42 (s, 1H).

LC-MS (ESI⁺): 188 (M+H)⁺; Retention time: 0.93 min (methode C)

### Example 2-1

### N-{[2,2-difluoro-1,3-benzodioxol-5-yl]methyl}-N′-[(7R)-7-hydroxy-5,6,7,8-tetrahydronaphthalen-1-yl]urea

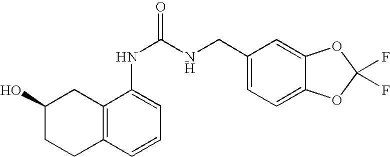

Phenyl-[(7R)-7-hydroxy-5,6,7,8-tetrahydronaphthalen-1-yl]carbamate (100 mg, 0.35 mmol) and 1-[2,2-difluoro-1,3-benzodioxol-5-yl]methanamine (66 mg, 0.35 mmol) are dissolved in dimethylsulfoxide (2.00 ml) and stirred at room temperature for 1 h. The raw material is purified via HPLC.

Yield: 47 mg (35%)

¹H NMR (300 MHz DMSO-d₆) δ 1.52-1.64 (m, 1H), 1.84-1.89 (m, 1H), 2.34 (dd, 1H), 2.64-2.87 (m, 3H), 3.91-3.92 (m, 1H), 4.29 (d, 2H), 4.82 (d, 1H), 6.72 (d, 1H), 6.98 (t, 1H), 7.05 (t, 1H), 7.15 (dd, 1H), 7.33-7.37 (m, 2H), 7.60-7.62 (m, 2H).

LC-MS (ESI⁺): 377.1 (M+H)⁺; Retention time: 2.00 min (method C)

### Example 2-2

### N-{[3-chloro-5-trifluoromethyl)pyridin-2-yl]methyl}-N′-[(7R)-7-hydroxy-5,6,7,8-tetrahydronaphthalen-1-yl]urea

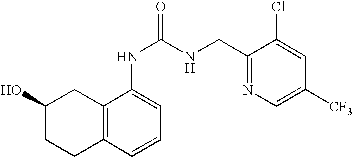

Phenyl-[(7R)-7-hydroxy-5,6,7,8-tetrahydronaphthalen-1-yl]carbamate (300 mg, 1.06 mmol), 1-[3-chloro-5-(trifluoromethyl)pyridin-2-yl]methanamine hydrochloride (261 mg, 1.06 mmol) and N,N-diisopropylethylamine (191 mg, 1.48 mmol) are dissolved in dimethylsulfoxide (2.00 ml). The mixture is reacted at 60° C. for 3 h, partitioned between ethyl acetate and water, the organic layer is dried over magnesium sulfate and evaporated to dryness in vacuo. The raw material is triturated with diethyl ether, filtered and dried.

Yield: 347 mg (82%)

¹H NMR (200 MHz, DMSO-d₆) δ 1.45-1.68 (m, 1H), 1.78-1.95 (m, 1H), 2.27-2.95 (m, 4H), 3.82-4.03 (m, 1H), 4.62 (d, 2H), 4.86 (d, 1H), 6.72 (d, 1H), 6.98 (t, 1H), 7.28 (t, 1H), 7.60 (d, 1H), 7.95 (s, 1H), 8.48 (d, 1H), 8.93 (d, 1H).

MS (ESI⁺): 400.1 (M+H)⁺

HPLC: Retention time 4.1 min (method B)

### Chapter III (Examples)

### Preparing Method of Starting Compounds

### [Starting Compound A]

7-ethoxy-5,8-dihydronaphthalen-1-ylamine

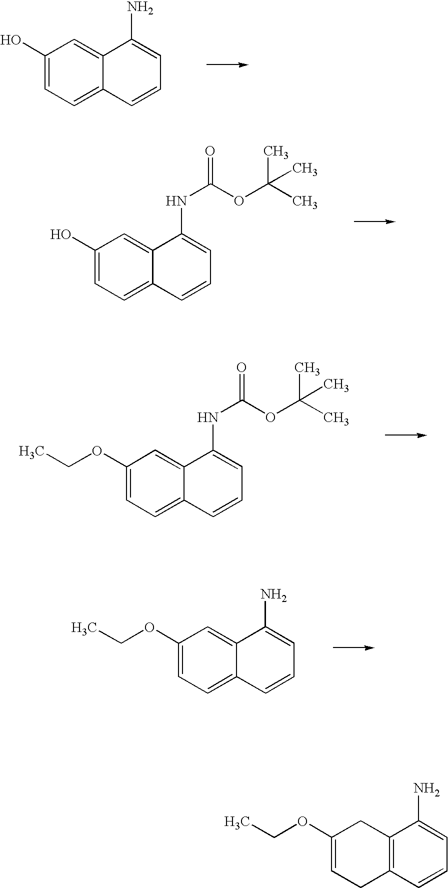

To a stirred solution of 8-amino-2-naphthol (50.0 g, 314 mmol) in tetrahydrofuran (1000 mL) was added di-t-butyldicarbonate (68.6 g, 314 mmol). The mixture was stirred at 70° C. for 18 hours. After the mixture was cooled to room temperature, solvent was removed under reduced pressure. To the residue was added ethylacetate, and washed with saturated aqueous solution of sodium carbonate and then with water. The extracted organic layer was dried over Na₂SO₄, filtered, and concentrated under reduced pressure. To the obtained residue was added diisopropyl ether, and the precipitate was filtered and dried to afford N-t-butoxycarbonyl-8-amino-2-naphthol (64.2 g, 79% yield).

Next, to a mixture of N-t-butoxycarbonyl-8-amino-2-naphthol (64.0 g, 247 mmol) and Cesium carbonate (161 g, 493 mmol) in 300 mL anhydrous DMF was added iodoethane (42.3 g, 272 mmol) at room temperature. The mixture was stirred at 60° C. for 2 hours. Water was added to the mixture, and the product was extracted with ethylacetate. The organic layer was washed with water and brine, dried over Na₂SO₄, filtered and concentrated under reduced pressure. To the obtained residue was added diisopropyl ether and the precipitate was collected and dried to afford (7-ethoxy-naphthalen-1-yl)-carbamic acid t-butyl ester (47.9 g, 67.5% yield).

Next, to a (7-ethoxy-naphthalen-1-yl)-carbamic acid t-butyl ester (47.9 g, 167 mmol) in 100 mL anhydrous 1,4-dioxane was added 4N HCl in 1,4-dioxane (100 mL) at 0° C. The mixture was stirred at room temperature for 2 hours. Diisopropyl ether was added to the reaction mixture and the precipitate was filtered. To the obtained solid was added saturated sodium bicarbonate and the product was extracted with ethylacetate. The organic layer was dried over Na₂SO₄, filtered, and concentrated under reduced pressure to afford 7-ethoxy-naphthalen-1-ylamine (27.0 g, 86.3% yield).

Next, to a flask containing a mixture of 7-ethoxy-naphthalen-1-ylamine (1.80 g, 9.61 mmol) and t-buthanol (2.13 g, 28.8 mmol) in tetrahydrofuran (20 mL) was collected liquid ammonia (300 mL) at −78° C. To the mixture was added lithium (0.200 g, 28.8 mmol) over 30 minutes and stirred at −78° C. for 1 hour. Methanol and water was added, and the mixture was stirred at room temperature for 16 hours to allow ammonia to evaporate. To the obtained residue was added ethylacetate. The organic layer was washed with water, dried over Na₂SO₄, filtered, and concentrated under reduced pressure to afford 7-ethoxy-5,8-dihydronaphthalen-1-ylamine (1.37 g, 76% yield).

### [Starting Compound B]

8-amino-1,2,3,4-tetrahydro-naphthalen-2-ol

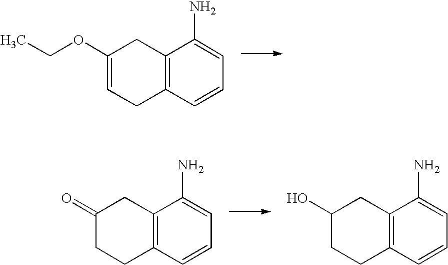

To a stirred solution of 7-ethoxy-5,8-dihydronaphthalen-1-ylamine (1.07 g, 5.65 mmol) in tetrahydrofuran (30 mL) was added solution of aqueous 2N HCl (10 mL), and stirred at 40° C. for 1 hour. The mixture was neutralized with addition of sodium bicorbonate, and the product was extracted with ethylacetate. The organic layer was washed with water, dried over Na₂SO₄, filtered, and concentrated under reduced pressure to afford 8-amino-3,4-dihydro-1H-naphthalen-2-one (0.71 g, 78% yield).

Next, to 8-amino-3,4-dihydro-1H-naphthalen-2-one (0.050 g, 0.318 mmol) in methanol (10 mL) was added sodium borohydride (0.030 g, 0.175 mmol) at 0° C., and the mixture was stirred for 1 hour. The mixture was poured into water, and the product was extracted with ethylacetate. The organic layer was dried over Na₂SO₄, filtered, and concentrated under reduced pressure to afford 8-amino-1,2,3,4-tetrahydro-naphthalen-2-ol (0.037 g, 71% yield).

### [Starting Compound C]

### 8-Amino-1,2,3,4-tetrahydro-naphthalen-2-ol (enantiomer)

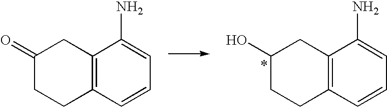

To a stirred solution of benzeneruthenium(II) chloride dimer (3.10 mg, 0.006 mmol) and (1S, 2R)-(−)-cis-1-amino-2-indanol (3.7 mg, 0.025 mmol) in degaussed isopropanol was heated at 80° C. for 20 minutes under argon. The mixture was added to the solution of 8-amino-3,4-dihydro-1H-naphthalen-2-one (50 mg, 0.310 mmol) in isopropanol (3 mL) at room temperature. A solution of potassium hydroxide (3.48 mg, 0.062 mmol) in isopropanol (1 mL) was added, and the mixture was stirred at 45° C. for 1 hour. The mixture was passed through silica gel and washed with ethylacetate. The filtrate was concentrated under reduced pressure to afford 8-amino-1,2,3,4-tetrahydro-naphthalen-2-ol enantiomer (33.0 mg, 65% yield).

The other enantiomer of 8-amino-1,2,3,4-tetrahydronaphthalen-2-ol was obtained in the same fashion replacing (1S,2R)-(−)-cis-1-amino-2-indanol with (1R,2S)-(+)-cis-1-amino-2-indanol.

### Example 1-1

### 5-chloro-N-(7-hydroxy-5,6,7,8-tetrahydronaphthalen-1-yl)-1H-indole-2-carboxamide

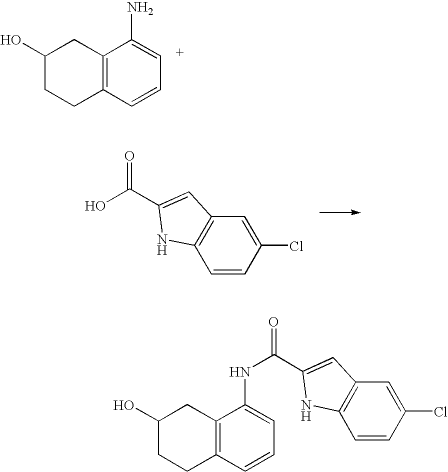

To 8-amino-1,2,3,4-tetrahydronaphthalen-2-ol (25.0 mg, 0.15 mmol) in tetrahydrofuran (2 mL) was added 5-chloro-1H-indole-2-carboxylic acid (30.0 mg, 0.15 mmol), 1,1′-carbonyldi(1,2,4-triazole) (31.6 mg, 0.15 mmol), and pyridine (12.1 mg, 0.15 mmol) at room temperature. After the mixture was stirred for 5 hours, water was added and then extracted with ethylacetate. The organic layer was dried over MgSO4, filtered, and concentrated under reduced pressure. The obtained residue was washed with diethylether to provide 5-chloro-N-(7-hydroxy-5,6,7,8-tetrahydronaphthalen-1-yl)-1H-indole-2-carboxamide (10.3 mg).

Molecular weight: 340.81

MS (ESI) m/z 341 [M+H]⁺

Melting Point: 254.3

Activity Class: B

In the similar manner as described in Example 1-1, compounds in Example 1-2 to 1-4 as shown in Table 1 were synthesized.

TABLE 1

| example | | | MS | | activity |
|---|---|---|---|---|---|
| # | structure | M.W. | (M + 1) | MP | class |
| 1-2 | 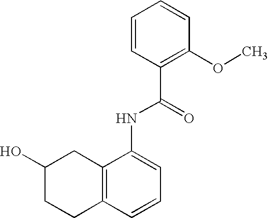 | 297,36 | 298 | 184-186 | C |
| 1-3 | 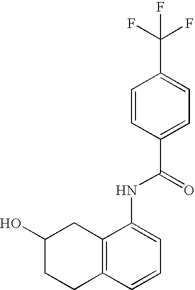 | 335,33 | 336 | 217-218 | B |
| 1-4 | 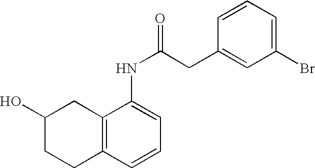 | 360,25 | 361 | 189 decomp. | C |

### [Starting Compound D]

2-bromo-N-(7-hydroxy-5,6,7,8-tetrahydronaphthalen-1-yl)acetamide

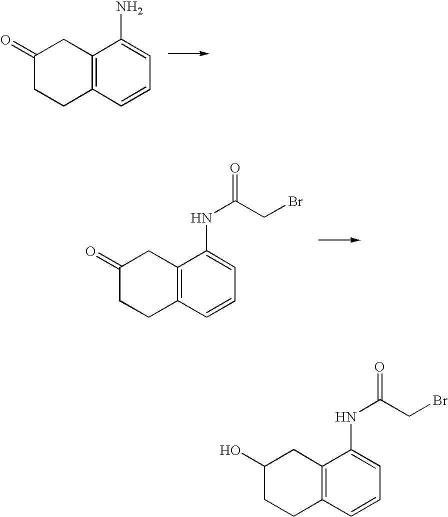

To a mixture of 8-amino-3,4-dihydronaphthalen-2(1H)-one (1.67 g, 20.2 mmol) and pyridine (0.949 g, 12.0 mmol) in tetrahydrofuran (80 mL) was added bromoacetyl chloride (1.73 g, 11.0 mmol) in tetrahydrofuran (20 mL) at 0° C. After the mixture was stirred for 2 hours at room temperature, water (50 mL) was added and extracted with ethylacetate. The organic layer was dried over MgSO4, filtered, and concentrated under reduced pressure. The obtained residue was purified by silica gel column chromatography (eluent: ethylacetate/hexane=1/2) to provide 2-bromo-N-7-oxo-5,6,7,8-tetrahydronaphthalen-1-yl)acetamide (2.18 g).

Molecular weight: 282.14

MS (ESI): m/z 283 [M+H]⁺

¹H NMR (CDCl₃-d) δ 2.48 (t, J=6.0 Hz, 2H), 3.05 (t, J=6.0 Hz, 2H), 3.47 (s, 2H), 4.30 (s, 2H), 7.14-7.28 (m, 3H), 9.76 (brs, 1H).

To a solution of 2-bromo-N-(7-oxo-5,6,7,8-tetrahydronaphthalen-1-yl)acetamide (564 mg, 2.00 mmol) in methanol (10 mL) was added sodium borohydride at 0° C. After the mixture was stirred for 30 minutes, water (2 mL) was added and then concentrated under reduced pressure. The resulting residue was mixed with tetrahydrofuran and filtered. The filtrate was concentrated under reduced pressure to afford 2-bromo-N-(7-hydroxy-5,6,7,8-tetrahydronaphthalen-1-yl)acetamide (558 mg).

Molecular weight: 284.15

MS (ESI) m/z 285 [M+H]⁺

### [Example 2-1]

### 2N-[4-chloro-3-(trifluoromethyl)phenyl]-1N-(7-hydroxy-5,6,7,8-tetrahydronaphthalen-1-yl)glycinamide

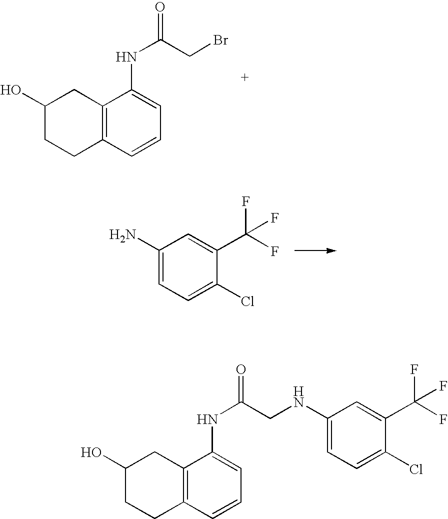

A mixture of 2-bromo-N-(7-hydroxy-5,6,7,8-tetrahydronaphthalen-1-yl)acetamide (141 mg, 0.50 mmol) and 4-chloro-3-trifluoromethylaniline (93.9 mg, 0.48 mmol) in dimethylsulfoxide (7 mL) was stirred at room temperature for 16 hours. To the reaction mixture was added potassium carbonate (138 mg, 1.00 mmol) and stirred at 50° C. for 48 hours. The mixture was poured into water and extracted with ethyl acetate. The organic layer was dried over MgSO4, filtered, and concentrated under reduced pressure. The obtained residue was purified by silica gel column chromatography (eluent: ethyl acetate/hexane=1/1) to give 2N-[4-chloro-3-(trifluoromethyl)phenyl]-1N-(7-hydroxy-5,6,7,8-tetrahydronaphthalen-1-yl)glycinamide (28.1 mg).

Molecular weight: 398.82

MS (ESI) m/z 399 [M+H]⁺

HPLC Retention Time: 4.45 minutes (Method A)

Activity Class: A

### [Starting Compound E]

ethyl-N-methyl-N-[4-(trifluoromethoxy)phenyl]glycinate

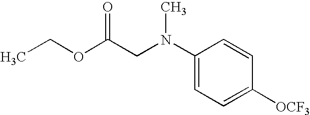

N-Methyl-4-trifluoromethoxyaniline (100 mg, 0.52 mmol), ethyl bromoacetate (262 mg, 1.57 mmol) and sodium carbonate (166 mg, 1.57 mmol) are reacted in dimethylacetamide (5 ml) at 60° C. over night. The reaction mixture is partitioned between ethyl acetate and water, the organic layer is dried over magnesium sulfate and evaporated to dryness in vacuo. The raw material is purified by preparative HPLC with an acetonitrile/water gradient.

Yield: 106 mg (73%)

¹H NMR (400 MHz, DMSO-d₆) δ 1.15 (t, 3H), 2.98 (s, 3H), 4.10 (q, 2H), 4.21 (s, 2H), 6.70 (d, 2H), 7.12 (d, 2H).

MS (ESI⁺): 278.1 [M+H]⁺

HPLC: Retention time 4.9 min (method B).

### [Starting Compound F]

### N-methyl-N-[4-(trifluoromethoxy)phenyl]glycine

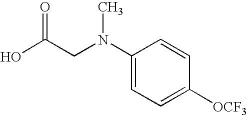

Ethyl-N-methyl-N-[4-(trifluoromethoxy)phenyl]glycinate (200 mg, 0.72 mmol) and potassium hydroxide (81 mg, 1.44 mmol) are dissolved in methanol/water (3 ml/1 ml) and stirred for 1 h at room temperature. The reaction mixture is acidified with 0.5 N hydrochloric acid to pH=3 and partitioned between ethyl acetate and water. The organic extracts are dried over magnesium sulfate and evaporated to dryness in vacuo. The raw material is purified by preparative chromatography on silica (eluent: ethyl acetate/methanol, 1:0-5:1).

Yield: 35 mg (18%)

¹H NMR (300 MHz, DMSO-d₆) δ 2.97 (s, 3H), 4.05 (s, 2H), 6.67 (d, 2H), 7.12 (d, 2H).

MS (ESI⁻): 247.9 [M−H]⁻

HPLC: Retention time 4.2 min (method B).

### [Starting Compound G]

2-bromo-N-[(7R)-7-hydroxy-5,6,7,8-tetrahydronaphthalen-1-yl]acetamide

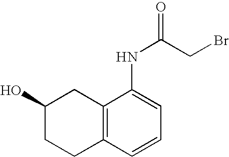

(2R)-8-Amino-1,2,3,4-tetrahydro-naphthalen-2-ol (1.20 g, 7.35 mmol) is dissolved in ethyl acetate (38 ml). Saturated aqueous sodium hydrogencarbonate (19 ml) is added, the mixture is stirred vigorously and bromoacetyl chloride (1.16 g, 7.35 mmol) is added slowly. Stirring continues for 10 minutes, the aqueous layer is separated and the organic layer is dried over magnesium sulfate, filtered and evaporated to dryness. The raw material is triturated with diethyl ether, filtered and dried in vacuo.

Yield: 1.65 g (79%)

¹H NMR (200 MHz, DMSO-d₆) δ 1.48-1.70 (m, 1H), 1.78-1.95 (m, 1H), 2.41 (dd, 1H), 2.60-2.95 (m, 3H), 3.80-3.98 (m, 1H), 4.08 (s, 2H), 4.84 (br s, 1H), 6.93 (d, 1H), 7.08 (t, 1H), 7.18 (d, 1H), 9.61 (s, 1H).

MS (ESI⁺): 301 [M+NH₄]⁺

HPLC: Retention time 3.40 min (method B).

### [Starting Compound E]

5-[4-trifluoromethoxy)phenyl]pyridine-2-carboxylic acid

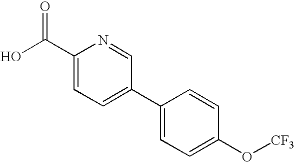

Under an argon atmosphere, to 4 ml 1,2-dimethoxyethane are added 5-bromo-pyridine-2-carboxylic acid (93 mg, 0.46 mmol), [4-(trifluoromethoxy)phenyl]boronic acid (114 mg, 0.55 mmol), 0.51 ml of a 2M aqueous sodium carbonate solution and dichlorobis-(triphenylphosphin)palladium(II) (20 mg, 0.03 mmol). The mixture is stirred at 90° C. overnight, cooled and quenched with water. Ethyl acetate is added and the mixture adjusted to pH=2 with 1N hydrochloric acid. After threefold extraction with ethyl acetate, the combined organic layers are dried over magnesium sulfate, filtered, and evaporated in vacuo. The residue is purified by silica gel chromatography (eluent: dichloromethane/methanol 5:1).

Yield: 56 mg (43%).

MS (ESI⁻): 282 [M−H]⁻

HPLC: Retention time 4.01 min (method B)

### [Starting Compound I]

5-[4-trifluoromethyl)phenyl]pyridine-2-carboxylic acid

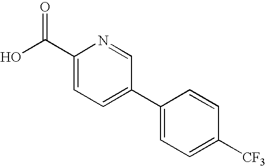

The compound is obtained accordingly to the procedure for starting compound H from 5-bromopyridine-2-carboxylic acid (93 mg, 0.46 mmol) and [4-(trifluoromethyl)phenyl]boronic acid (105 mg, 0.55 mmol).

Yield: 76 mg (62%).

LC-MS (ESI⁺): 268 [M+H]⁺; Retention time: 1.97 min (method E)

### [Starting Compound J]

methyl 6-[4-trifluoromethoxy)phenyl]nicotinate

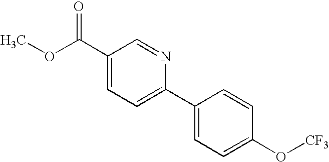

Under an argon atmosphere, to 4 ml 1,2-dimethoxyethane are added methyl 6-chloronicotinate (230 mg, 1.06 mmol), [4-(trifluoromethoxy)phenyl]boronic acid (268 mg, 1.31 mmol), 1.28 ml of a 2M aqueous sodium carbonate solution and tetrakis-(triphenylphosphin)palladium(0) (62 mg, 0.05 mmol). The mixture is stirred at 80° C. for 16 h, cooled and quenched with water. After threefold extraction with ethyl acetate, the combined organic layers are washed with brine, dried over magnesium sulfate, filtered, and evaporated in vacuo. The residue is purified by silica gel chromatography (eluent: cyclohexane/ethyl acetate 7:1).

Yield: 180 mg (57%).

¹H NMR (400 MHz, DMSO-d₆) δ 3.92 (s, 3H), 7.53 (d, 2H), 8.17 (d, 1H), 8.30 (d, 2H), 8.40 (dd, 1H), 9.18 (d, 1H).

MS (ESI⁺): 298 [M+H]⁺

HPLC: Retention time 5.01 min (method B)

### [Starting Compound K]

methyl 6-[4-(trifluoromethyl)phenyl]nicotinate

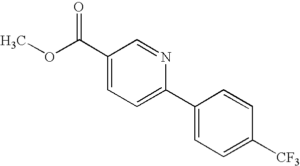

The compound is obtained accordingly to the procedure for starting compound J from methyl 6-chloronicotinate (1.00 g, 5.83 mmol) and [4-(trifluoromethyl)phenyl]boronic acid (1.33 g, 6.99 mmol).

Yield: 1.06 mg (65%).

¹H NMR (300 MHz, DMSO-d₆) δ 3.92 (s, 3H), 7.90 (d, 2H), 8.25 (dd, 1H), 8.38 (d, 2H), 8.42 (dd, 1H), 9.21 (dd, 1H).

MS (ESI⁺): 282 [M+R]⁺

HPLC: Retention time 4.88 min (method B)

### [Starting Compound L]

6-[4-(trifluoromethoxy)phenyl]nicotinic acid

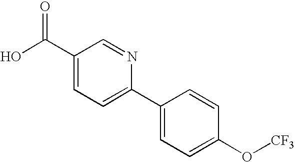

Methyl 6-[4-(trifluoromethoxy)phenyl]nicotinate (170 mg, 0.57 mmol) and powdered potassium hydroxide (96 mg, 1.72 mmol) are dissolved in 2 ml methanol and 0.05 ml water. After stirring the mixture at 40° C. overnight, the methanol is evaporated in vacuo. The residue is taken up with water and ethyl acetate and the aqueous phase is adjusted to pH=2 with 1N hydrochloric acid. After threefold extraction with ethyl acetate, the combined organic layers are washed with brine, dried over magnesium sulfate, and evaporated. The remaining residue is treated with diethyl ether, filtered, washed with diethyl ether and dried.

Yield: 148 mg (91%).

¹H NMR (400 MHz, DMSO-d₆) δ 7.58 (d, 2H), 8.21 (d, 1H), 8.36 (d, 2H), 8.42 (dd, 1H), 9.22 (d, 1H) 13.50 (s, 1H).

MS (ESI⁺): 284 (M+H)⁺

HPLC: Retention time 4.33 min (method B)

### [Starting Compound M]

6-[4-trifluoromethyl)phenyl]nicotinic acid

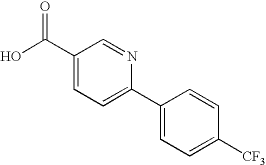

The compound is obtained accordingly to the procedure for starting compound L from methyl 6-[4-(trifluoromethyl)phenyl]nicotinate (250 mg, 0.89 mmol).

Yield: 212 mg (89%).

¹H NMR (400 DMSO-d₆) δ 7.90 (d, 2H), 8.23 (d, 1H), 8.35-8.42 (m, 3H,), 9.19 (d, 1H), 13.3-13.7 (broad s, 1H).

MS (ESI⁺): 268 [M+H]⁺

HPLC: Retention time 4.40 min (method B)

### Example 3-1

### N-[(7R)-7-hydroxy-5,6,7,8-tetrahydronaphthalen-1-yl]-N2-methyl-N2-[4-(trifluoromethoxy)phenyl]glycinamide

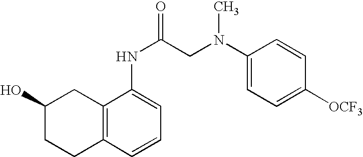

(2R)-8-Amino-1,2,3,4-tetrahydro-naphthalen-2-ol (21 mg, 0.13 mmol), N′-(3-dimethylaminopropyl)-N-ethylcarbodiimide hydrochloride (32 mg, 0.17 mmol), 1-hydroxy-1H-benzotriazole (21 mg, 0.15 mmol) and N-methyl-N-[4-(trifluoromethoxy)phenyl]glycine (35 mg, 0.14 mmol) are dissolved in dimethylacetamide (3 ml). The reaction mixture is stirred over night at room temperature, partitioned between ethyl acetate and water, dried over magnesium sulfate and evaporated to dryness in vacuo. The raw material is purified by chromatography on silica (eluent: cyclohexane/ethyl acetate, 1:1).

Yield: 24 mg (45%).

¹H NMR (300 MHz, DMSO-d₆) δ 1.5-1.65 (m, 1H), 1.78-1.90 (m, 1H), 2.40 (dd, 1H), 2.62-2.90 (m, 3H), 3.32-3.44 (m, 1H), 4.18 (s, 2H), 6.75 (d, 2H), 6.90 (d, 1H), 7.04 (t, 1H), 7.12-7.22 (m, 3H), 9.20 (s, 1H).

MS (ESI⁺): 395.0 [M+H]⁺

HPLC: Retention time 4.4 min (method B)

### Example 3-2

### N-[(7R)-7-hydroxy-5,6,7,8-tetrahydronaphthalen-1-yl]-N²-[4-(trifluoromethoxy)phenyl]glycinamide

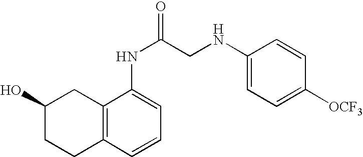

2-Bromo-N-[(7R)-7-hydroxy-5,6,7,8-tetrahydronaphthalen-1-yl]acetamide (100 mg, 0.35 mmol), 4-trifluoromethoxyaniline (62 mg, 0.35 mmol) and triethylamine (71 mg, 0.70 mmol) are dissolved in dry dimethylformamide (2 ml) and stirred at 60° C. for 2 h. The mixture is partitioned between ethyl acetate and water, the organic layer is dried over magnesium sulfate and evaporated to dryness. The raw material is purified by preparative chromatography on silica (eluent: cyclohexane/ethyl acetate, 2:1-0:1).

Yield: 8 mg (6%).

¹H NMR (200 MHz, DMSO-d₆) δ 1.40-1.70 (m, 1H), 1.73-1.92 (m, 1H), 2.35 (dd, 1H), 2.55-2.95 (m, 3H), 3.80-4.05 (m, 3H), 4.80 (d, 1H), 6.38 (t, 1H), 6.66 (d, 2H), 6.90 (d, 1H), 7.00-7.20 (m, 3H), 7.27 (d, 1H), 9.23 (s, 1H).

MS (ESI⁺): 381.3 [M+H]⁺

HPLC: Retention time 4.35 min (method B).

### Example 3-3

### N-[(7R)-7-hydroxy-5,6,7,8-tetrahydronaphthalen-1-yl]-4-(trifluoromethoxy)benzamide

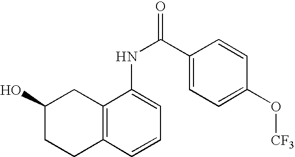

(2R)-8-Amino-1,2,3,4-tetrahydro-naphthalen-2-ol (70 mg, 0.43 mmol), N′-(3-dimethylaminopropyl)-N-ethylcarbodiimide hydrochloride (107 mg, 0.56 mmol), 1-hydroxy-1H-benzotriazole hydrate (70 mg, 0.52 mmol) and 4-trifluoromethoxybenzoic acid (97 mg, 0.47 mmol) are dissolved in dimethylformamide (3 ml). The reaction mixture is stirred over night at room temperature and then evaporated to dryness in vacuo. The raw material is solved in DMSO and purified by HPLC.

Yield: 96 mg (64%).

¹H NMR (300 MHz, DMSO-d₆) δ 1.73-1.83 (m, 1H), 2.02-2.15 (m, 1H), 2.92-3.19 (m, 3H), 3.98-4.12 (m, 2H), 5.00 (d, 1H), 7.19-7.33 (m, 3H), 7.72 (d, 1H), 8.29 (d, 2H), 10.12 (s, 1H).

LC-MS (ESI⁺): 352.1 [M+H]⁺; Retention time: 2.88 min (method G)

### Example 3-4

### N-[(7R)-7-hydroxy-5,6,7,8-tetrahydronaphthalen-1-yl](trifluoromethyl)benzamide

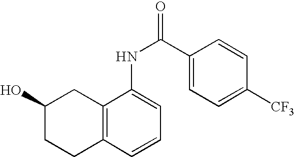

(2R)-8-Amino-1,2,3,4-tetrahydro-naphthalen-2-ol (70 mg, 0.43 mmol), N′-(3-dimethylaminopropyl)-N-ethylcarbodiimide hydrochloride (107 mg, 0.56 mmol), 1-hydroxy-1H-benzotriazole hydrate (70 mg, 0.52 mmol) and 4-trifluoromethylbenzoic acid (90 mg, 0.47 mmol) are dissolved in dimethylformamide (3 ml). The reaction mixture is stirred over night at room temperature and then evaporated to dryness in vacuo. The raw material is solved in DMSO and purified by HPLC.

Yield: 110 mg (76%).

¹H NMR (300 MHz, DMSO-d₆) δ 1.55-1.67 (m, 1H), 1.87-1.92 (m, 1H), 2.48 (dd, 1H), 2.72-2.96 (m, 3H), 3.87-3.91 (m, 1H), 4.75 (dd, 1H), 7.15-7.01 (m, 3H), 7.91 (d, 2H), 8.16 (d, 2H), 10.00 (s, 1H).

LC-MS (ESI⁺): 336.1 [M+H]⁺; Retention time: 2.84 min (method G)

### Example 3-5

### N-[(7R)-7-hydroxy-5,6,7,8-tetrahydronaphthalen-1-yl]-2-[4-(trifluoromethyl)phenylacetamide

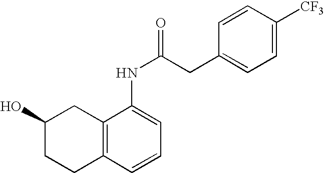

(2R)-8-Amino-1,2,3,4-tetrahydro-naphthalen-2-ol (70 mg, 0.43 mmol), N′-(3-dimethylaminopropyl)-N-ethylcarbodiimide hydrochloride (107 mg, 0.56 mmol), 1-hydroxy-1H-benzotriazole hydrate (70 mg, 0.52 mmol) and [4-(trifluoromethyl)phenyl]acetic acid (96 mg, 0.47 mmol) are dissolved in dimethylformamide (3 ml). The reaction mixture is stirred over night at room temperature and then evaporated to dryness in vacuo. The raw material is solved in DMSO and purified by HPLC.

Yield: 108 mg (72%).

¹H NMR (300 MHz, DMSO-d₆) δ 1.53-1.65 (m, 1H), 1.83-1.88 (m, 1H), 2.42 (dd, 1H), 2.66-2.91 (m, 3H), 3.80 (s, 2H), 3.86-3.90 (m, 1H), 4.77 (d, 1H), 6.91 (d, 1H), 7.04 (t, 1H), 7.18 (d, 1H), 7.57 (d, 2H), 7.70 (d, 1H), 9.43 (s, 1H).

LC-MS (ESI⁺): 350.1 [M+H]⁺; Retention time: 2.86 min (method G)

### Example 3-6

### N-[(7R)-7-hydroxy-5,6,7,8-tetrahydronaphthalen-1-yl]-3-[4-(trifluoromethyl)phenyl]propanamide

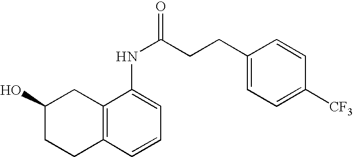

(2R)-8-Amino-1,2,3,4-tetrahydro-naphthalen-2-ol (70 mg, 0.43 mmol), N′-(3-dimethylaminopropyl)-N-ethylcarbodiimide hydrochloride (107 mg, 0.56 mmol), 1-hydroxy-1H-benzotriazole hydrate (70 mg, 0.52 mmol) and [4-(trifluoromethyl)phenyl]propanoic acid (103 mg, 0.47 mmol) are dissolved in dimethylformamide (3 ml). The reaction mixture is stirred over night at room temperature and then evaporated to dryness in vacuo. The raw material is solved in DMSO and purified by HPLC.

Yield: 85 mg (55%).

¹H NMR (300 MHz, DMSO-d₆) δ 1.52-1.64 (m, 1H), 1.82-1.87 (m, 1H), 2.37 (dd, 1H), 2.66-2.90 (m, 5H), 3.01 (t, 2H), 3.84 (m, 1H), 4.74 (d, 1H), 6.89 (d, 1H), 7.03 (t, 1H), 7.13 (d, 1H), 7.50 (d, 2H), 7.65 (d, 1H), 9.14 (s, 1H).

LC-MS (ESI⁺): 364.1 [M+H]⁺; Retention time: 2.97 min (method G)

### Example 3-7

### N-[(7R)-7-hydroxy-5,6,7,8-tetrahydronaphthalen-1-yl]-2-[4-(trifluoromethoxy)phenylacetamide

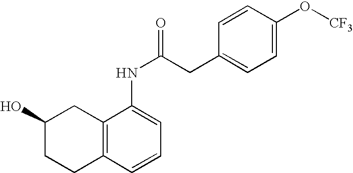

(2R)-8-Amino-1,2,3,4-tetrahydro-naphthalen-2-ol (100 mg, 0.61 mmol), N′-(3-dimethylaminopropyl)-N-ethylcarbodiimide hydrochloride (153 mg, 0.80 mmol), 1-hydroxy-1H-benzotriazole hydrate (99 mg, 0.74 mmol) and [4-(trifluoromethoxy)phenyl]acetic acid (148 mg, 0.67 mmol) are dissolved in dimethylformamide (3 ml). The reaction mixture is stirred over night at room temperature and then evaporated to dryness in vacuo. The raw material is solved in DMSO and purified by HPLC.

Yield: 170 mg (76%).

¹H NMR (300 MHz, DMSO-d₆) δ 1.51-1.65 (m, 1H), 1.76-1.92 (m, 1H), 2.41 (dd, 1H), 2.79-2.87 (m, 3H), 3.72 (s, 2H), 3.81-3.94 (m, 1H), 4.85 (d, 1H), 6.91 (d, 1H), 7.05 (t, 1H), 7.17 (d, 1H), 7.33 (d, 2H), 7.45 (d, 1H), 9.44 (s, 1H).

LC-MS (ESI⁺): 366.0 [M+H]⁺; Retention time: 2.08 min (method F)

### Example 3-8

### 2-(4-chlorophenoxy)-N-[(7R)-7-hydroxy-5,6,7,8-tetrahydronaphthalen-1-yl]acetamide

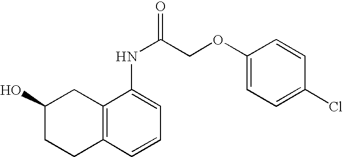

Under an argon atmosphere, (2R)-8-amino-1,2,3,4-tetrahydro-naphthalen-2-ol (150 mg, 0.92 mmol), N′-3-dimethylaminopropyl)-N-ethylcarbodiimide hydrochloride (229 mg, 1.19 mmol), 1-hydroxy-1H-benzotriazole (149 mg, 1.10 mmol) and (4-chlorophenoxy)acetic acid (189 mg, 1.01 mmol) are added to 2 ml DMF at room temperature and the reaction is stirred overnight. Water is then added and the resulting mixture is extracted with ethyl acetate three times. The combined organic phases are washed with brine, dried over magnesium sulfate and evaporated in vacuo. The residue is purified first by chromatography on silica gel (eluent cyclohexane/ethyl acetate 2:1), then by preparative reversed phase HPLC (eluent water/acetonitrile gradient). After collecting the appropriate product fractions and evaporating the solvent in vacuo, the residue is washed thoroughly with diethyl ether and dried to give the target compound

Yield: 227 mg (74%).

¹H NMR (400 MHz, DMSO-d₆) δ 1.53-1.65 (m, 1H), 1.81-1.91 (m, 1H), 2.42 (dd, 1H), 2.73 (ddd, 1H), 2.84 (dd, 1H), 2.88 (dd, 1H), 3.84-3.94 (m, 1H), 4.72 (s, 2H), 4.81 (d, 1H), 6.95 (d, 1H), 7.05 (d, 2H), 7.09 (d, 1H), 7.21 (d, 1H), 7.37 (d, 2H), 9.39 s, 1H).

MS (ESI⁺): 332 [M+H]⁺

HPLC: Retention time 4.23 min (method B)

### Example 3-9

### 2-(2,4-difluorophenoxy)-N-[(7R)-7-hydroxy-5,6,7,8-tetrahydronaphthalen-1-yl]acetamide

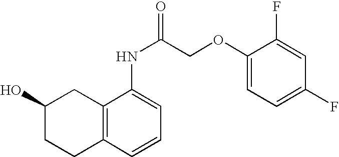

The compound is obtained accordingly to the procedure for Example 3-8 from (2R)-8-amino-1,2,3,4-tetrahydro-naphthalen-2-ol (150 mg, 0.92 mmol) and (2,4-difluorophenoxy)acetic acid (190 mg, 1.01 mmol).

Yield: 199 mg (65%)

¹H NMR (400 MHz, DMSO-d₆) δ 1.53-1.66 (m, 1H), 1.81-1.92 (m, 1H), 2.42 (dd, 1H), 2.73 (ddd, 1H), 2.84 (dd, 1H), 2.88 (dd, 1H), 3.84-3.94 (m, 1H), 4.80 (s, 2H), 4.81 (d, 1H), 6.94 (d, 1H), 7.00-7.12 (m, 2H), 7.20 (dt, 1H), 7.26 (d, 1H), 7.33 (ddd, 1H) 9.35 (s, 1H).

MS (ESI⁺): 334 [M+H]⁺

HPLC: Retention time 4.11 min (method B)

### Example 3-10

### 2-[2-chloro-4-(trifluoromethyl)phenoxy]-N-[(7R)-7-hydroxy-5,6,7,8-tetrahydronaphthalen-1-yl]acetamide

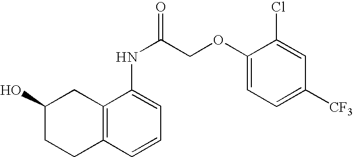

The compound is obtained accordingly to the procedure for Example 3-8 from (2R)-8-amino-1,2,3,4-tetrahydro-naphthalen-2-ol (80 mg, 0.49 mmol) and [2-chloro-4-(trifluoromethyl)phenoxy]acetic acid (137 mg, 0.54 mmol).

Yield: 150 mg (77%).

¹H NMR (x00 MHz, DMSO-d₆) δ 1.55-1.67 (m, 1H), 1.82-1.92 (m, 1H), 2.46 (dd, 1H), 2.73 (ddd, 1H), 2.86 (dd, 1H), 2.90 (dd, 1H), 3.86-3.96 (m, 1H), 4.84 (d, 1H), 4.50 (s, 2H), 6.94 (d, 1H), 7.09 (t, 1H), 7.31(d, 1H), 7.35 (d, 1H), 7.72 (dd, 1H), 7.89 (d, 1H), 9.34 (s, 1H).

MS (ESI⁺): 400 M+H]⁺

HPLC: Retention time 4.64 min (method D)

### Example 3-11

### N-[(7R)-7-hydroxy-5,6,7,8-tetrahydronaphthalen-1-yl]-2-[4-(trifluoromethyl)phenoxy]-acetamide

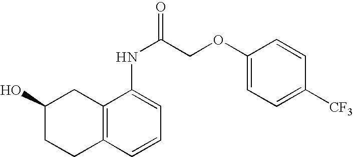

The compound is obtained accordingly to the procedure for Example 3-8 (omitting the first chromatography over silica gel) from (2R)-8-amino-1,2,3,4-tetrahydro-naphthalen-2-ol (100 mg, 0.61 mmol) and [4-(trifluoromethyl)phenoxy]acetic acid (148 mg, 0.67 mmol).

Yield: 153 mg (68%).

¹H NMR (400 MHz, DMSO-d₆) δ 1.54-1.66 (m, 1H), 1.81-1.92 (m, 1H), 2.43 (dd, 1H), 2.73 (ddd, 1H), 2.81-2.92 (m, 2H), 3.84-3.94 (m, 1H), 4.81 (d, 1H), 4.83 (s, 2H), 6.96 (d, 1H), 7.08 (t, 1H), 7.16-7.24 (m, 2H), 7.70 (d, 1H), 9.46 (s, 1H).

MS (CI⁺): 383 [M+NH₄]⁺

HPLC: Retention time 4.38 min (method B)

### Example 3-12

### N-[(7R)-7-hydroxy-5,6,7,8-tetrahydronaphthalen-1-yl]-2-[4-(trifluoromethoxy)phenoxy]-acetamide

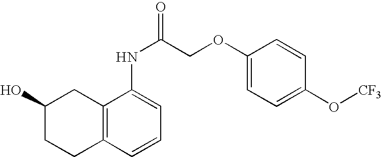

The compound is obtained accordingly to the procedure for Example 3-8 (the crude reaction mixture is applied to reversed phase HPLC purification directly) from (2R)-8-amino-1,2,3,4-tetrahydro-naphthalen-2-ol (80 mg, 0.49 mmol) and [4-(trifluoromethoxy)phenoxy]acetic acid (127 mg, 0.54 mmol).

Yield: 119 mg (64%).

¹H NMR (400 MHz, DMSO-d₆) δ 1.54-1.66 (m, 1H), 1.81-1.91 (m, 1H), 2.42 (dd, 1H), 2.73 (ddd, 1H), 2.84 (dd, 1H), 2.88 (dd, 1H), 3.84-3.94 (m, 1H), 4.75 (s, 2H), 0.81 (d, 1H), 6.96 (d, 1H), 7.09 (t, 1H), 7.12 (d, 1H), 7.21 (d, 1H), 7.34 (d, 1H), 9.41 (s, 1H).

MS (CI⁺): 399 [M+NH₄]⁺

HPLC: Retention time 4.44 min (method B)

### Example 3-13

### N-[(7R)-7-hydroxy-5,6,7,8-tetrahydronaphthalen-1-yl]-5-[4-(trifluoromethoxy)phenyl]-pyridine-2-carboxamide

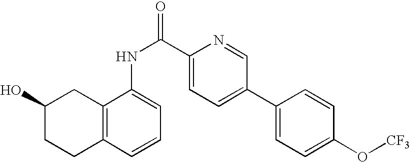

The compound is obtained accordingly to the procedure for Example 3-8 (the crude reaction mixture is applied to reversed phase HPLC purification directly) from (2R)-8-amino-1,2,3,4-tetrahydro-naphthalen-2-ol (25 mg, 0.15 mmol) and 5-[4-(trifluoromethoxy)phenyl]pyridine-2-carboxylic acid (50 mg, 0.18 mmol).

Yield: 20 mg (31%).

¹H NMR (400 MHz, DMSO-d₆) δ 1.41-1.52 (m, 1H), 1.68-1.76 (m, 1H), 2.37 (dd, 1H), 2.59 (dd, 1H), 2.74 (dt, 1H), 2.81 (dd, 1H), 3.75-3.85 (m, 1H), 4.71 (d, 1H), 6.79 (d, 1H), 6.99 (t, 1H), 7.38 (d, 2H), 7.53-7.61 (m, 1H), 7.82 (d, 2H), 8.07 (d, 1H), 8.22 (dd, 1H), 8.91 (d, 1H), 10.01 (s, 1H).

LC-MS (ESI⁺): 429 [M+H]⁺; Retention time: 281 min (method E)

### Example 3-14

### N-[(7R)-7-hydroxy-5,6,7,8-tetrahydronaphthalen-1-yl]-5-[4-(trifluoromethyl)phenyl]-pyridine-2-carboxamide

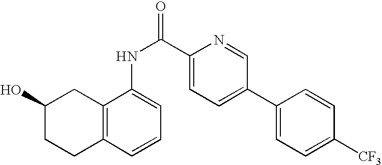

The compound is obtained accordingly to the procedure for Example 3-8 (the crude reaction mixture is applied to reversed phase HPLC purification directly) from (2R)-8-amino-1,2,3,4-tetrahydro-naphthalen-2-ol (42 mg, 0.26 mmol) and 5-[4-(trifluoromethyl)phenyl]pyridine-2-carboxylic acid (75 mg, 0.28 mmol).

Yield: 29 mg (27%).

¹H NMR (300 MHz, DMSO-d₆) δ 1.57-1.72 (m, 1H), 1.85-1.96 (m, 1H), 2.56 (dd, 1H), 2.77 (ddd, 1H), 2.91 (dt, 1H), 2.99 (dd, 1H), 3.92-4.04 (m, 1H), 4.86 (d, 1H), 6.97 (d, 1H), 7.16 (t, 1H), 7.74 (d, 1H), 7.91 (d, 2H), 8.09 (d, 1H), 8.27 (d, 1H), 8.45 (dd, 1H), 9.13 (d, 1H), 10.19 (s, 1H).

MS (ESI⁺): 413 [M+H]⁺

HPLC: Retention time 4.88 min (method D)

### Example 3-15

### N-[(7R)-7-hydroxy-5,6,7,8-tetrahydronaphthalen-1-yl]-6-[4-(trifluoromethyl)phenyl]-nicotinamide

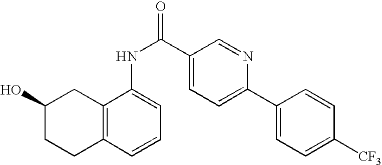

The compound is obtained accordingly to the procedure for Example 3-8 (the crude reaction mixture is applied to reversed phase HPLC purification directly) from (2R)-8-amino-1,2,3,4-tetrahydro-naphthalen-2-ol (117 mg, 0.71 mmol) and 6-[4-(trifluoromethyl)phenyl]nicotinic acid (210 mg, 0.79 mmol).

Yield: 230 mg (78%).

¹H NMR (400 MHz, DMSO-d₆) δ 1.46-1.57 (m, 1H), 1.75-1.84 (m, 1H), 2.36-2.56 (dd., 1H), 2.68 (ddd, 1H), 2.76-2.88 (m, 2H), 3.75-3.85 (m, 1H), 4.69 (d, 1H), 6.93 (d, 1H), 7.01-7.11 (m, 2H), 7.80 (d, 2H), 8.17 (d, 1H), 8.30 (d, 2H), 8.36 (dd, 1H), 9.16 (s, 1H), 9.95 (s, 1H).

MS (ESI⁺): 413 [M+H]⁺

HPLC: Retention time 4.42 min (method B)

### Example 3-16

### N-[(7R)-7-hydroxy-5,6,7,8-tetrahydronaphthalen-1-yl]-6-[4-(trifluoromethoxy)phenyl]nicotinamide

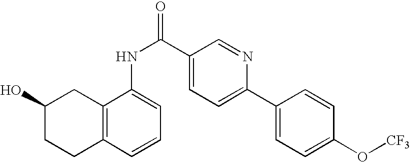

The compound is obtained accordingly to the procedure for Example 3-8 (the crude reaction mixture is applied to reversed phase HPLC purification directly) from (2R)-8-amino-1,2,3,4-tetrahydro-naphthalen-2-ol (37 mg, 0.23 mmol) and 6-[4-(trifluoromethoxy)phenyl]nicotinic acid (71 mg, 0.25 mmol).

Yield: 67 mg (69%).

¹H NMR (400 MHz, DMSO-d₆) δ 1.63-1.75 (m, 1H), 1.92-2.01 (m, 1H), 2.54-2.63 (dd, 1H), 2.85 (ddd, 1H), 2.94-3.04 (m, 2H), 3.92-4.01 (m, 1H), 4.86 (d, 1H), 7.11 (d, 1H), 7.18-7.28 (m, 2H), 7.60 (d, 2H), 8.27 (d, 1H), 8.39 (d, 2H), 8.49 (dd, 1H), 9.30 (s, 1H), 10.10 (s, 1H).

MS (ESI⁺): 429 [M+H]⁺

HPLC: Retention time 4.40 min (method B)

### Chapter IV (Examples)

### Preparing Method of Compounds

### [Starting Compound A]

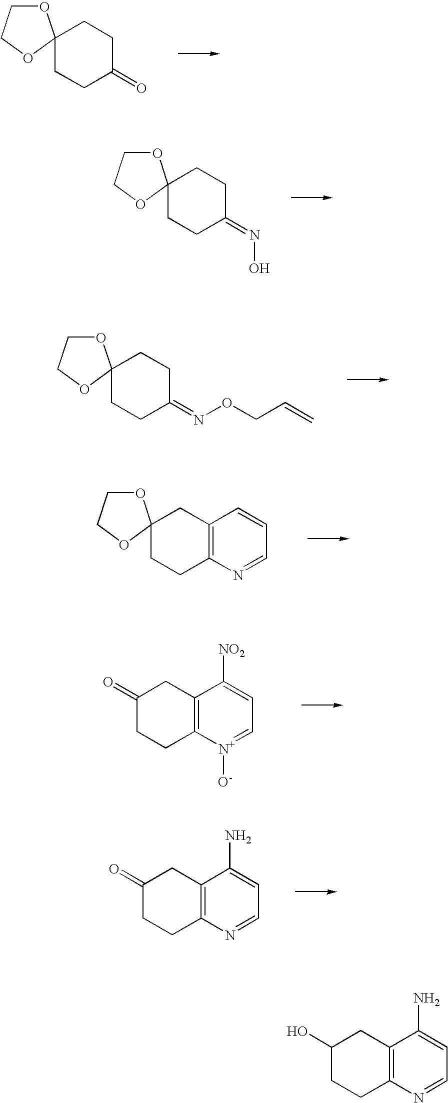

A mixture of 1,4-dioxaspiro[4.5]decan-8-one (3.12 g, 20.0 mmol), hydroxylamine hydrochloride (1.67 g, 24.0 mmol), and triethylamine (2.42 g, 24.0 mmol) in methanol (50 mL) was stirred under reflux for 2 hours. The resulting mixture was concentrated under reduced pressure and then purified by silica gel column chromatography (eluent: ethylacetate/hexane=1/1) to provide 1,4-dioxaspiro[4.5]decan-8-one oxime (2.73 g).

Molecular weight: 171.20

MS (ESI) m/z 172 [M+H]⁺

¹H NMR (CDCl₃-d) δ 1.26 (t, J=7.2 Hz, 2H), 1.76 (t, J=7.2 Hz, 2H), 2.41 (t, J=6.5 Hz, 2H), 2.68 (t, J=6.5 Hz, 2H), 3.99 (s, 4H), 7.80 (brs, 1H).

Next to a mixture of 1,4-dioxaspiro[4.5]decan-8-one oxime (2.73 g, 16.0 mmol), allyl bromide (5.79 g, 47.8 mmol), and potassium carbonate (4.41 g, 31.9 mmol) in acetone (100 mL) was stirred under reflux for 15 hours. After the mixture was cooled to ambient temperature, it was filtered and the filtrate was concentrated under reduced pressure. The obtained residue was purified by silica gel column chromatography (eluent: ethylacetate/hexane=1/4) to give 1,4-dioxaspiro-[4.5]decan-8-one O-allyloxime (1.01 mg).

Molecular weight: 211.26

MS (ESI) m/z 212 [M+H]⁺

¹H NMR (CDCl₃-d) δ 1.76 (t, J=7.0 Hz, 2H), 1.82 (t, J=7.0 Hz, 2m), 2.40 (t, J=6.5 Hz, 2H), 2.66 (t, J=6.5 Hz, 2H), 3.98 (s, 4H), 4.53 (dd, J=1.3, 4.3 Hz, 2H), 5.20 (dd, J=1.3, 10.4 Hz, 1H), 5.30 (d, J=10.4 Hz, 1H), 5.96-6.02 (m, 1H).

Next, 1,4-dioxaspiro[4.5]decan-8-one O-allyloxime (1.00 mg, 4.78 mmol) was heated neat at 230° C. for 21 hours. After the residue was cooled to ambient temperature, it was purified by silica gel column chromatography (eluent: tetrahydrofuran/hexane=1/2) to afford 7′,8′-dihydro-5′H-spiro[1,3-dioxolane-2,6′-quinoline] (105 mg).

Molecular weight: 191.23

MS (ESI) m/z 192 [M+H]⁺

¹H NMR (CDCl₃-d) δ 2.05 (t, J=6.9 Hz, 2H), 3.15 (t, J=6.9 Hz, 2H), 3.00 (s, 2H), 4.06 (s, 4H), 7.05 (dd, J=4.8, 7.7 Hz, 1H), 7.34 (d, J=7.7 Hz, 1H), 8.39 (d, J=4.8 Hz, 1H).

Next, 7′,8′-dihydro-5′H-spiro[1,3-dioxolane-2,6′-quinoline] is treated with a mixture of nitric acid and sulfuric acid and then the mixture is heated to reflux. After cooled to room temperature, water is added and the mixture is extracted with ethyl acetate. Concentration of the organic layer under reduced pressure yields 4-nitro-7,8-dihydroquinolin-6(5H)-one 1-oxide.

Next, a solution of 4-nitro-7,8-dihydroquinolin-6(5H)-one 1-oxide in tetrahydrofuran is treated under hydrogen atmosphere in the presence of catalytic amount of Pt/C. The mixture is passed through celite and is concentrated under reduced pressure to give 4-amino-7,8-dihydroquinolin-6(5H)-one.

A solution of 4-amino-7,8-dihydroquinolin-6(5H)-one in tetrahydrofuran is treated with sodium borohydride. After stirring for 6 hours, water is added. The mixture is extracted with ethyl acetate, dried and the organic layer is then concentrated under reduced pressure. The resulting residue is purified by silica gel column chromatography to give 4-amino-5,6,7,8-tetrahydroquinolin-6-ol.

### Example 1-1

### N-[4-chloro-3-(trifuoromethyl)phenyl]-N′-(6-hydroxy-5,6,7,8-tetrahydroquinolin-4-yl)urea

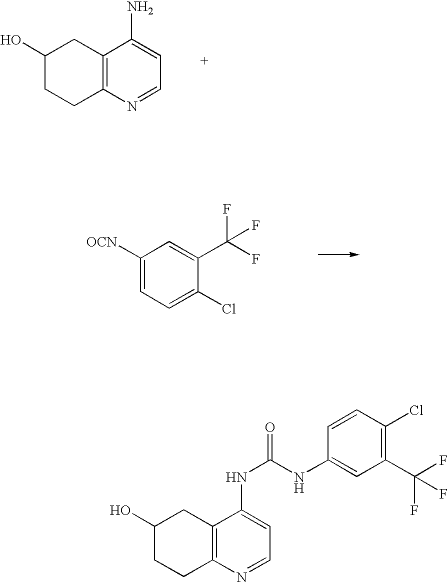

A mixture of 4-amino-5,6,7,8-tetrahydroquinolin-6-ol and 4-chloro-3-trifluoromethylphenyl isocyanate in tetrahydrofuran is stirred at 50° C. for 5 hours. After removing the solvent, the resulting residue is purified by silica gel column chromatography to provide N-[4-chloro-3-(trifluoromethyl)phenyl]-N′-(6-hydroxy-5,6,7,8-tetrahydroquinolinyl)urea.

In a similar manner as described in Example 1-1, Example 1-2 to 1-8 as shown in Table 1 are synthesized.

Also, Example 2-1 to 2-8 as shown in Table 2, Example 3-1 to 3-8 as shown in Table 3, and Example 4-1 to 4-8 as shown in Table 4 are synthesized in a similar manner as described in Example 1-1.

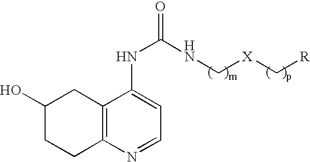

| Exam- | | | | |
|---|---|---|---|---|
| ple | m | —X— | p | —R |
| 1-2 | 1 | bond | 0 | 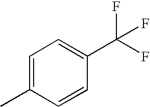 |
| 1-3 | 0 | bond | 0 | 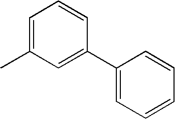 |
| 1-4 | 0 | bond | 0 | 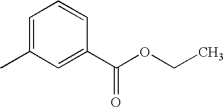 |
| 1-5 | 1 | bond | 0 | 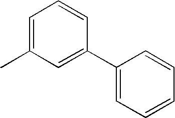 |
| 1-6 | 2 | —O— | 0 | 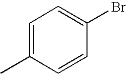 |
| 1-7 | 2 | —N(C₂H₅)— | 0 | 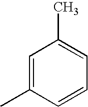 |
| 1-8 | 2 | —N(CH₃)— | 0 | 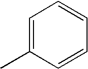 |

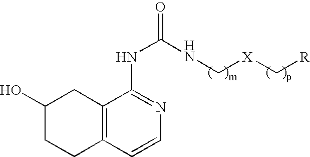

| Exam- | | | | |
|---|---|---|---|---|
| ple | m | —X— | p | —R |
| 2-1 | 0 | bond | 0 | 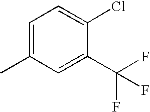 |
| 2-2 | 1 | bond | 0 | 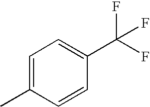 |
| 2-3 | 0 | bond | 0 | 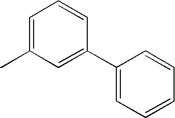 |
| 2-4 | 0 | bond | 0 | 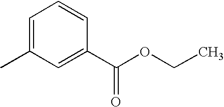 |
| 2-5 | 1 | bond | 0 | 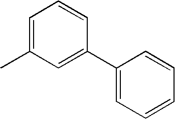 |
| 2-6 | 2 | —O— | 0 | 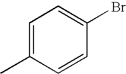 |
| 2-7 | 2 | —N(C₂H₅)— | 0 | 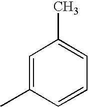 |
| 2-8 | 2 | —N(CH₃)— | 0 | 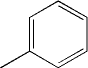 |

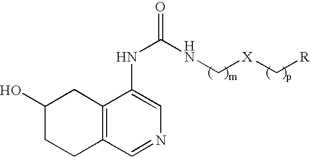

| Exam- | | | | |
|---|---|---|---|---|
| ple | m | —X— | p | —R |
| 3-1 | 0 | bond | 0 | 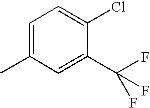 |
| 3-2 | 1 | bond | 0 | 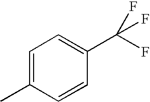 |
| 3-3 | 0 | bond | 0 | 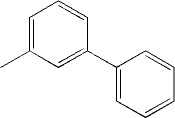 |
| 3-4 | 0 | bond | 0 | 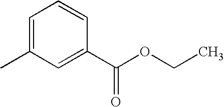 |
| 3-5 | 1 | bond | 0 | 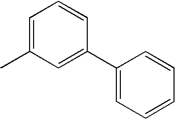 |
| 3-6 | 2 | —O— | 0 | 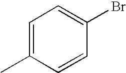 |
| 3-7 | 2 | —N(C₂H₅)— | 0 | 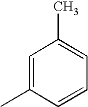 |
| 3-8 | 2 | —N(CH₃)— | 0 | 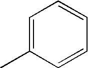 |

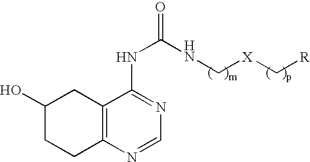

| Exam- | | | | |
|---|---|---|---|---|
| ple | m | —X— | p | —R |
| 4-1 | 0 | bond | 0 | 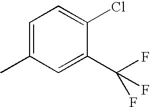 |
| 4-2 | 1 | bond | 0 | 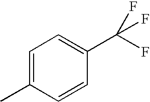 |
| 4-3 | 0 | bond | 0 | 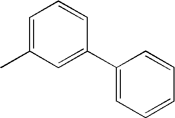 |
| 4-4 | 0 | bond | 0 | 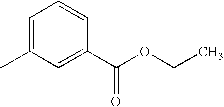 |
| 4-5 | 1 | bond | 0 | 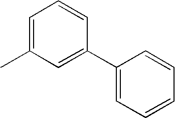 |
| 4-6 | 2 | —O— | 0 | 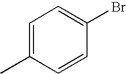 |
| 4-7 | 2 | —N(C₂H₅)— | 0 | 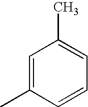 |
| 4-8 | 2 | —N(CH₃)— | 0 | 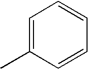 |

1. A compound of the formula (A), their tautomeric and stereoisomeric form, and salts thereof: 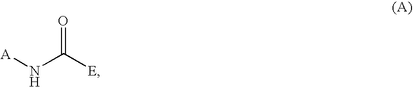
wherein
A represents the formula 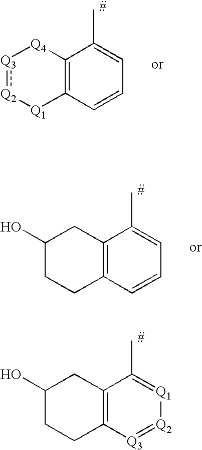
wherein
# represents the connection position to the molecule,
Q₁ and Q₄ (Chapter I) independently represent direct bond or methylene;
Chemical bond between [symbol]
(Chapter I) is selected from the group consisting of a single bond and a double bond;
when [symbol]
(Chapter I) is a single bond, Q₂ (Chapter I) represents CHR², or CO, and Q₃ (Chapter I) represents CHR³,
when [symbol]
(Chapter I) is a double bond, Q₂ (Chapter I) represents CR² and Q₃ (Chapter I) represents CR³;
wherein
R² (Chapter I) represents hydrogen, hydroxy, C₁₋₆ alkoxy or C₁₋₆ alkanoyloxy;
R³ (Chapter I) represents hydrogen, hydroxy, C₁₋₆ alkoxy, C₁₋₆ alkanoyloxy, or C₁₋₆ alkyl optionally substituted by hydroxy, C₁₋₆ alkoxy or C₁₋₆ alkanoyloxy,
with the proviso that Q₁ and Q₄ (Chapter I) can not be direct bond at the same time;
R² and R³ (Chapter I) can not be hydrogen at the same time;
when Q₁ and Q₄ (Chapter I) are both methylene and R³ (Chapter I) is hydroxy, R² (Chapter I) is hydroxy, C₁₋₆ alkoxy or C₁₋₆ alkanoyloxy;
when Q₁ (Chapter I) is direct bond, R² (Chapter I) is hydroxy, C₁₋₆ alkoxy or C₁₋₆ alkanoyloxy; and when Q₄ (Chapter I) is direct bond, R² (Chapter I) is hydrogen, C₁₋₆ alkoxy or C₁₋₆ alkanoyloxy;
Q₁, Q₂ and Q₃ (Chapter IV) independently represent N or CH,
with the proviso that at least one of Q₁, Q₂ and Q₃ (Chapter IV) is N;
and
E represents the formula 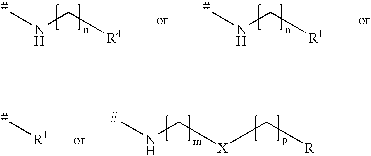
wherein
# represents the connection position to the molecule
n represents an integer of 0 to 6;
R⁴ represents aryl optionally having one or two substituents selected from the group consisting of halogen, hydroxy, C₁₋₆alkylamino, di(C₁₋₆ alkyl)amino, C₃₋₈ cycloalkylamino, C₁₋₆ alkoxycarbonyl, phenyl, benzyl, sulfonamide, C₁₋₆ alkanoyl, C₁₋₆ alkanoylamino, carbamoyl, C₁₋₆ alkylcarbamoyl, cyano, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen, phenoxy optionally substituted by halogen or C₁₋₆ alkyl, and C₁₋₆ alkylthio optionally substituted by mono-, di-, or tri-halogen,
R¹ (Chapter II) represents C₃₋₈cycloalkyl optionally fused by aryl,
wherein
said aryl is optionally substituted with one or more substituents selected from the group consisting of halogen, hydroxy, carboxy, nitro, cyano, amino, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen and C₁₋₆ alkylthio optionally substituted by mono-, di-, or tri-halogen;
phenyl substituted by heteroaryl, or heteroaryloxy,
wherein
said heteroaryl and heteroaryloxy are optionally substituted with one or more substituents selected from the group consisting of halogen, hydroxy, carboxy, nitro, cyano, amino, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen, and C₁₋₆ alkylthio optionally substituted by mono-, di-, or tri-halogen;
phenyl fused with heteroaryl, or heterocyclyl,
wherein
said heteroaryl is optionally substituted with one or more substituents selected from the group consisting of halogen, hydroxy, carboxy, nitro, cyano, amino, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen and C₁₋₆ alkylthio optionally substituted by mono-, di-, or tri-halogen;
or
heteroaryl optionally substituted with one or more substituents selected from the group consisting of halogen, hydroxy, carboxy, nitro, cyano, amino, phenyl, benzyl, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen and C₁₋₆ alkylthio optionally substituted by mono-, di-, or tri-halogen,
R¹ (Chapter III) represents aryl or heteroaryl,
wherein
said aryl and heteroaryl are optionally substituted with one or more substituents selected from the group consisting of halogen, nitro, hydroxy, carboxy, amino, C₁₋₆ alkylamino, di(C₁₋₆alkyl)amino, C₃₋₈ cycloalkylamino, C₁₋₆ alkoxycarbonyl, phenyl (which phenyl is optionally substituted by halogen, trifluoromethyl, trifluoromethoxy, nitro, hydroxy, carboxy, amino, C₁₋₆alkylamino, di(C₁₋₆alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆alkyl)amino, C₃₋₈ cycloalkylamino, or C₁₋₆ alkoxycarbonyl or C₁₋₆ alkyl,), C₁₋₆ alkylthio (which alkylthio is optionally substituted by mono-, di-, or tri-halogen), C₃₋₈ cycloalkyl, and heterocycle;
C₁₋₆ alkyl optionally substituted by R¹¹, OR¹², SR¹² or N(R¹²)(R¹³),
wherein
R¹¹ represents aryl or heteroaryl,
wherein
said aryl and heteroaryl are optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle;
R¹² represents aryl, heteroaryl, or C₁₋₆ alkyl optionally substituted by aryl or heteroaryl,
wherein
said aryl and heteroaryl are optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
R¹³ represents hydrogen, or C₁₋₆ alkyl;
or
C₃₋₈cycloalkyl optionally fused by aryl,
wherein
said aryl is optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl), amino, C₃₋₈ cycloalkylamino, or C₁₋₆ alkoxycarbonyl), heterocycle, sulfonamide, C₁₋₆ alkanoyl, C₁₋₆ alkanoylamino, carbamoyl, C₁₋₆ alkylcarbamoyl, cyano, C₁₋₆alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl and heterocycle,
m represents 0, 1, 2, or 3;
p represents 0 or 1;
—X— represents a bond, —O— or —N(R¹)— (wherein R¹ is hydrogen or C₁₋₆ alkyl);
with the proviso that when m is 0, —X— represents a bond,
R represents aryl or heteroaryl,
wherein said aryl and heteroaryl are optionally substituted with one or more substituents independently selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

2. Compound of formula (A) according to claim 1, with the formula (I), their tautomeric and stereoisomeric form, and salts thereof: 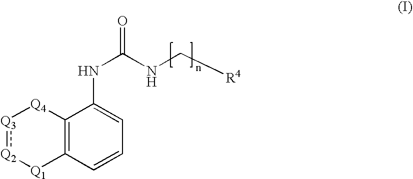
wherein
n represents an integer of 0 to 6;
Q₁ and Q₄ independently represent direct bond or methylene;
Chemical bond between [symbol]
is selected from the group consisting of a single bond and a double bond;
when [symbol]
is a single bond, Q₂ represents CHR², or CO, and Q₃ represents CHR³,
when [symbol]
is a double bond, Q₂ represents CR² and Q₃ represents CR³;
wherein
R² represents hydrogen, hydroxy, C₁₋₆ alkoxy or C₁₋₆ alkanoyloxy;
R³ represents hydrogen, hydroxy, C₁₋₆ alkoxy, C₁₋₆ alkanoyloxy, or C₁₋₆ alkyl optionally substituted by hydroxy, C₁₋₆ alkoxy or C₁₋₆alkanoyloxy,
with the proviso that Q₁ and Q₄ can not be direct bond at the same time;
R² and R³ can not be hydrogen at the same time;
when Q₁ and Q₄ are both methylene and R³ is hydroxy, R² is hydroxy, C₁₋₆ alkoxy or C₁₋₆ alkanoyloxy;
when Q₁ is direct bond, R² is hydroxy, C₁₋₆ alkoxy or C₁₋₆ alkanoyloxy; and when Q₄ is direct bond, R² is hydrogen, C₁₋₆ alkoxy or C₁₋₆alkanoyloxy;
and
R⁴ represents aryl optionally having one or two substituents selected from the group consisting of halogen, hydroxy, C₁₋₆ alkylamino, di(C₁₋₆ alkyl)amino, C₃₋₈ cycloalkylamino, C₁₋₆ alkoxycarbonyl, phenyl, benzyl, sulfonamide, C₁₋₆ alkanoyl, C₁₋₆ alkanoylamino, carbamoyl, C₁₋₆ alkylcarbamoyl, cyano, C₁₋₆alkyl optionally substituted by cyano, C₁₋₆ alkoxycarbonyl or mono-, di-, or tri-halogen, C₁₋₆alkoxy optionally substituted by mono-, di-, or tri-halogen, phenoxy optionally substituted by halogen or C₁₋₆ alkyl, and C₁₋₆ alkylthio optionally substituted by mono-, di-, or tri-halogen.

3. Compound of formula (A) according to claim 1, with the formula (I), their tautomeric and stereoisomeric form, and salts thereof: 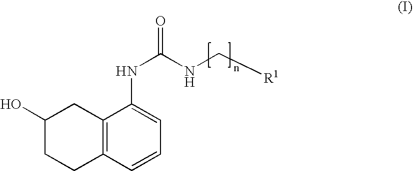
wherein
n represents an integer of 0 to 6; and
R¹ represents C₃₋₈cycloalkyl optionally fused by aryl,
wherein
said aryl is optionally substituted with one or more substituents selected from the group consisting of halogen, hydroxy, carboxy, nitro, cyano, amino, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen and C₁₋₆ alkylthio optionally substituted by mono-, di-, or tri-halogen;
phenyl substituted by heteroaryl, or heteroaryloxy,
wherein
said heteroaryl and heteroaryloxy are optionally substituted with one or more substituents selected from the group consisting of halogen, hydroxy, carboxy, nitro, cyano, amino, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆ alkoxycarbonyl, or mono-, di-, or tri-halogen, C₁₋₆alkoxy optionally substituted by mono-, di-, or tri-halogen, and C₁₋₆ alkylthio optionally substituted by mono-, di-, or tri-halogen;
phenyl fused with heteroaryl, or heterocyclyl,
wherein
said heteroaryl is optionally substituted with one or more substituents selected from the group consisting of halogen, hydroxy, carboxy, nitro, cyano, amino, C₁₋₆ alkylamino, di(C₁₋₆alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆alkoxycarbonyl, or mono-, di-, or tri-halogen, C₁₋₆ alkoxy optionally substituted by mono-, di-, or tri-halogen and C₁₋₆ alkylthio optionally substituted by mono-, di-, or tri-halogen;
or
heteroaryl optionally substituted with one or more substituents selected from the group consisting of halogen, hydroxy, carboxy, nitro, cyano, amino, phenyl, benzyl, C₁₋₆ alkylamino, di(C₁₋₆ alkyl)amino, C₁₋₆ alkoxycarbonyl, C₁₋₆ alkanoyl, C₁₋₆ alkanoylamino, carbamoyl, C₁₋₆ alkylcarbamoyl, C₁₋₆ alkyl optionally substituted by cyano, C₁₋₆ alkoxycarbonyl, or mono-, di-, or tri-halogen, C₁₋₆ alkoxy optionally substituted by mono-, di-, or tri-halogen and C₁₋₆ alkylthio optionally substituted by mono-, di-, or tri-halogen.

4. Compound of formula (A) according to claim 1, with the formula (I), their tautomeric and stereoisomeric form, and salts thereof: 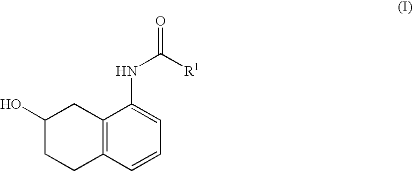
wherein
R¹ represents aryl or heteroaryl,
wherein
said aryl and heteroaryl are optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, trifluoromethyl, trifluoromethoxy, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆alkylthio (which alkylthio is optionally substituted by mono-, di-, or tri-halogen), C₃₋₈ cycloalkyl, and heterocycle;
C₁₋₆ alkyl optionally substituted by R¹¹, OR¹², SR¹² or N(R¹²)(R¹³),
wherein
R¹¹ represents aryl or heteroaryl,
wherein
said aryl and heteroaryl are optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle;
R¹² represents aryl, heteroaryl, or C₁₋₆ alkyl optionally substituted by aryl or heteroaryl,
wherein
said aryl and heteroaryl are optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle; and
R¹³ represents hydrogen, or C₁₋₆ alkyl;
or
C₃₋₈cycloalkyl optionally fused by aryl,
wherein
said aryl is optionally substituted with one or more substituents selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

5. Compound of formula (A) according to claim 1, with the formula (I), their tautomeric and stereoisomeric form, and salts thereof: 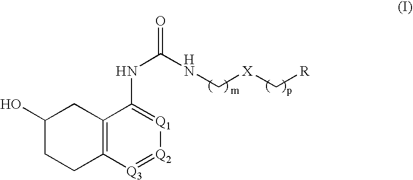
wherein
m represents 0, 1, 2, or 3;
p represents 0 or ;
—X— represents a bond, —O— or —N(R¹)— (wherein R¹ is hydrogen or C₁₋₆ alkyl);
with the proviso that when m is 0, —X— represents a bond.
Q₁, Q₂ and Q₃ independently represent N or CH,
with the proviso that at least one of Q₁, Q₂ and Q₃ is N;
R represents aryl or heteroaryl,
wherein said aryl and heteroaryl are optionally substituted with one or more substituents independently selected from the group consisting of halogen, nitro, hydroxy, carboxy, amino, C₁₋₆ alkylamino, di(C₁₋₆ alkyl)amino, C₃₋₈ cycloalkylamino, C₁₋₆ alkoxycarbonyl, phenyl (which phenyl is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), benzyl (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl), sulfonamide, C₁₋₆ alkanoyl, C₁₋₆ alkanoylamino, carbamoyl, C₁₋₆ alkylcarbamoyl, cyano, C₁₋₆ alkyl (which alkyl is optionally substituted by cyano, nitro, hydroxy, carboxy, amino, C₁₋₆ alkoxycarbonyl or mono-, di-, or tri-halogen), C₁₋₆ alkoxy (which alkoxy is optionally substituted by mono-, di-, or tri-halogen), phenoxy (in which phenyl moiety is optionally substituted by halogen, nitro, hydroxy, carboxy, amino, C₁₋₆ alkylamino, di(C₁₋₆ alkyl)amino, C₃₋₈ cycloalkylamino, or C₁₋₆ alkoxycarbonyl or C₁₋₆ alkyl), C₁₋₆ alkylthio (which alkylthio is optionally substituted by mono-, di-, or tri-halogen), C₃₋₈ cycloalkyl, and heterocycle.

6. A medicament comprising the compound of the formula (A), its tautomeric or stereoisomeric form, or a physiologically acceptable salt thereof as claimed in claim 1 as an active ingredient.

7. The medicament as claimed in claim 6, further comprising one or more pharmaceutically acceptable excipients.

8. The medicament as claimed in claim 6, wherein said compound of the formula (A), its tautomeric or stereoisomeric form, or a physiologically acceptable salt thereof is a VR1 antagonist.

9. The medicament as claimed in claim 6 for the treatment and/or prevention of an urological disorder or disease.

10. The medicament as claimed in claim 9, wherein said urological disorder or disease is detrusor overactivity (overactive bladder), urinary incontinence, neurogenic detrusor overactivity (detrusor hyperflexia), idiopathic detrusor overactivity (detrusor instability), benign prostatic hyperplasia, and lower urinary tract symptoms.

11. The medicament as claimed in claim 6 for the treatment and/or prevention of pain.

12. The medicament as claimed in claim 11, wherein said pain is chronic pain, neuropathic pain, postoperative pain, or rheumatoid arthritic pain.

13. The medicament as claimed in claim 6 for the treatment and/or prevention of a disorder or disease related to pain.

14. The medicament as claimed in claim 13, wherein said disorder or disease related to pain is neuralgia, neuropathies, algesia, nerve injury, ischaemia, neurodegeneration, or stroke.

15. The medicament as claimed in claim 6 for the treatment and/or prevention of an inflammatory disorder or disease.

16. The medicament as claimed in claim 15, wherein said inflammatory disorder or disease is asthma or COPD.

17. Use of compounds according to claim 1 for manufacturing a medicament for the treatment and/or prevention of an urological disorder or disease.

18. Use of compounds according to claim 1 for manufacturing a medicament for the treatment and/or prevention of pain.

19. Use of compounds according to claim 1 for manufacturing a medicament for the treatment and/or prevention of an inflammatory disorder or disease.

20. Process for controlling an urological disorder or disease in humans and animals by administration of a VR1-antagonistically effective amount of at least one compound according to claim 1.

21. Process for controlling pain in humans and animals by administration of a VR1-antagonistically effective amount of at least one compound according to claim 1.

22. Process for controlling an inflammatory disorder or disease in humans and animals by administration of a VR1-antagonistically effective amount of at least one compound according to claim 1.
